SEQUENCE LISTING (10 sequences)

SEQ ID NO: 1 - protein, 1213 aa, Homo sapiens
  VARIANT (44)..(44): X is I or V
  VARIANT (384)..(384): X is Y or H
  VARIANT (918)..(918): X is D or E
EDCNELPPRRNTEILTGSWSDQTYPEGTQAIYKCRPGYRSLGNXIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNXGRKFVQGKSIDVACHPGYALPKAQTTVTCMENGWSPTPRCIRVKTCSKSSIDIENGFISESQYTYALKEKAKYQCKLGYVTADGETSGSITCGKDGWSAQPTCIKSCDIPVFMNARTKNDFTWFKLNDTLDYECHDGYESNTGSTTGSIVCGYNGWSDLPICYERECELPKIDVHLVPDRKKDQYKVGEVLKFSCKPGFTIVGPNSVQCYHFGLSPDLPICKEQVQSCGPPPELLNGNVKEKTKEEYGHSEVVEYYCNPRFLMKGPNKIQCVDGEWTTLPVCIVEESTCGDIPELEHGWAQLSSPPYYYGDSVEFNCSESFTMIGHRSITCIHGVWTQLPQCVAIDKLKKCKSSNLIILEEHLKNKKEFDHNSNIRYRCRGKEGWIHTVCINGRWDPEVNCSMAQIQLCPPPPQIPNSHNMTTTLNYRDGEKVSVLCQENYLIQEGEEITCKDGRWQSIPLCVEKIPCSQPPQIEHGTINSSRSSQESYAHGTKLSYTCEGGFRISEENETTCYMGKWSSPPQCEGLPCKSPPXISHGVVAHMSDSYQYGEEVTYKCFEGFGIDGPAIAKCLGEKWSHPPSCIKTDCLSLPSFENAIPMGEKKDVYKAGEQVTYTCATYYKMDGASNVTCINSRWTGRPTCRDTSCVNPPTVQNAYIVSRQMSKYPSGERVRYQCRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKR

SEQ ID NO: 2 - protein, 1213 aa, Homo sapiens
EDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNYGRKFVQGKSIDVACHPGYALPKAQTTVTCMENGWSPTPRCIRVKTCSKSSIDIENGFISESQYTYALKEKAKYQCKLGYVTADGETSGSITCGKDGWSAQPTCIKSCDIPVFMNARTKNDFTWFKLNDTLDYECHDGYESNTGSTTGSIVCGYNGWSDLPICYERECELPKIDVHLVPDRKKDQYKVGEVLKFSCKPGFTIVGPNSVQCYHFGLSPDLPICKEQVQSCGPPPELLNGNVKEKTKEEYGHSEVVEYYCNPRFLMKGPNKIQCVDGEWTTLPVCIVEESTCGDIPELEHGWAQLSSPPYYYGDSVEFNCSESFTMIGHRSITCIHGVWTQLPQCVAIDKLKKCKSSNLIILEEHLKNKKEFDHNSNIRYRCRGKEGWIHTVCINGRWDPEVNCSMAQIQLCPPPPQIPNSHNMTTTLNYRDGEKVSVLCQENYLIQEGEEITCKDGRWQSIPLCVEKIPCSQPPQIEHGTINSSRSSQESYAHGTKLSYTCEGGFRISEENETTCYMGKWSSPPQCEGLPCKSPPEISHGVVAHMSDSYQYGEEVTYKCFEGFGIDGPAIAKCLGEKWSHPPSCIKTDCLSLPSFENAIPMGEKKDVYKAGEQVTYTCATYYKMDGASNVTCINSRWTGRPTCRDTSCVNPPTVQNAYIVSRQMSKYPSGERVRYQCRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKR

SEQ ID NO: 3 - protein, 1213 aa, Homo sapiens
EDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNHGRKFVQGKSIDVACHPGYALPKAQTTVTCMENGWSPTPRCIRVKTCSKSSIDIENGFISESQYTYALKEKAKYQCKLGYVTADGETSGSITCGKDGWSAQPTCIKSCDIPVFMNARTKNDFTWFKLNDTLDYECHDGYESNTGSTTGSIVCGYNGWSDLPICYERECELPKIDVHLVPDRKKDQYKVGEVLKFSCKPGFTIVGPNSVQCYHFGLSPDLPICKEQVQSCGPPPELLNGNVKEKTKEEYGHSEVVEYYCNPRFLMKGPNKIQCVDGEWTTLPVCIVEESTCGDIPELEHGWAQLSSPPYYYGDSVEFNCSESFTMIGHRSITCIHGVWTQLPQCVAIDKLKKCKSSNLIILEEHLKNKKEFDHNSNIRYRCRGKEGWIHTVCINGRWDPEVNCSMAQIQLCPPPPQIPNSHNMTTTLNYRDGEKVSVLCQENYLIQEGEEITCKDGRWQSIPLCVEKIPCSQPPQIEHGTINSSRSSQESYAHGTKLSYTCEGGFRISEENETTCYMGKWSSPPQCEGLPCKSPPEISHGVVAHMSDSYQYGEEVTYKCFEGFGIDGPAIAKCLGEKWSHPPSCIKTDCLSLPSFENAIPMGEKKDVYKAGEQVTYTCATYYKMDGASNVTCINSRWTGRPTCRDTSCVNPPTVQNAYIVSRQMSKYPSGERVRYQCRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKR

SEQ ID NO: 4 - protein, 1213 aa, Homo sapiens
EDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNYGRKFVQGKSIDVACHPGYALPKAQTTVTCMENGWSPTPRCIRVKTCSKSSIDIENGFISESQYTYALKEKAKYQCKLGYVTADGETSGSITCGKDGWSAQPTCIKSCDIPVFMNARTKNDFTWFKLNDTLDYECHDGYESNTGSTTGSIVCGYNGWSDLPICYERECELPKIDVHLVPDRKKDQYKVGEVLKFSCKPGFTIVGPNSVQCYHFGLSPDLPICKEQVQSCGPPPELLNGNVKEKTKEEYGHSEVVEYYCNPRFLMKGPNKIQCVDGEWTTLPVCIVEESTCGDIPELEHGWAQLSSPPYYYGDSVEFNCSESFTMIGHRSITCIHGVWTQLPQCVAIDKLKKCKSSNLIILEEHLKNKKEFDHNSNIRYRCRGKEGWIHTVCINGRWDPEVNCSMAQIQLCPPPPQIPNSHNMTTTLNYRDGEKVSVLCQENYLIQEGEEITCKDGRWQSIPLCVEKIPCSQPPQIEHGTINSSRSSQESYAHGTKLSYTCEGGFRISEENETTCYMGKWSSPPQCEGLPCKSPPDISHGVVAHMSDSYQYGEEVTYKCFEGFGIDGPAIAKCLGEKWSHPPSCIKTDCLSLPSFENAIPMGEKKDVYKAGEQVTYTCATYYKMDGASNVTCINSRWTGRPTCRDTSCVNPPTVQNAYIVSRQMSKYPSGERVRYQCRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKR

SEQ ID NO: 5 - protein, 1213 aa, Homo sapiens
EDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNHGRKFVQGKSIDVACHPGYALPKAQTTVTCMENGWSPTPRCIRVKTCSKSSIDIENGFISESQYTYALKEKAKYQCKLGYVTADGETSGSITCGKDGWSAQPTCIKSCDIPVFMNARTKNDFTWFKLNDTLDYECHDGYESNTGSTTGSIVCGYNGWSDLPICYERECELPKIDVHLVPDRKKDQYKVGEVLKFSCKPGFTIVGPNSVQCYHFGLSPDLPICKEQVQSCGPPPELLNGNVKEKTKEEYGHSEVVEYYCNPRFLMKGPNKIQCVDGEWTTLPVCIVEESTCGDIPELEHGWAQLSSPPYYYGDSVEFNCSESFTMIGHRSITCIHGVWTQLPQCVAIDKLKKCKSSNLIILEEHLKNKKEFDHNSNIRYRCRGKEGWIHTVCINGRWDPEVNCSMAQIQLCPPPPQIPNSHNMTTTLNYRDGEKVSVLCQENYLIQEGEEITCKDGRWQSIPLCVEKIPCSQPPQIEHGTINSSRSSQESYAHGTKLSYTCEGGFRISEENETTCYMGKWSSPPQCEGLPCKSPPDISHGVVAHMSDSYQYGEEVTYKCFEGFGIDGPAIAKCLGEKWSHPPSCIKTDCLSLPSFENAIPMGEKKDVYKAGEQVTYTCATYYKMDGASNVTCINSRWTGRPTCRDTSCVNPPTVQNAYIVSRQMSKYPSGERVRYQCRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKR

SEQ ID NO: 6 - protein, 1213 aa, Homo sapiens
EDCNELPPRRNTEILTGSWSDQTYPEGTQAIYKCRPGYRSLGNIIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNYGRKFVQGKSIDVACHPGYALPKAQTTVTCMENGWSPTPRCIRVKTCSKSSIDIENGFISESQYTYALKEKAKYQCKLGYVTADGETSGSITCGKDGWSAQPTCIKSCDIPVFMNARTKNDFTWFKLNDTLDYECHDGYESNTGSTTGSIVCGYNGWSDLPICYERECELPKIDVHLVPDRKKDQYKVGEVLKFSCKPGFTIVGPNSVQCYHFGLSPDLPICKEQVQSCGPPPELLNGNVKEKTKEEYGHSEVVEYYCNPRFLMKGPNKIQCVDGEWTTLPVCIVEESTCGDIPELEHGWAQLSSPPYYYGDSVEFNCSESFTMIGHRSITCIHGVWTQLPQCVAIDKLKKCKSSNLIILEEHLKNKKEFDHNSNIRYRCRGKEGWIHTVCINGRWDPEVNCSMAQIQLCPPPPQIPNSHNMTTTLNYRDGEKVSVLCQENYLIQEGEEITCKDGRWQSIPLCVEKIPCSQPPQIEHGTINSSRSSQESYAHGTKLSYTCEGGFRISEENETTCYMGKWSSPPQCEGLPCKSPPEISHGVVAHMSDSYQYGEEVTYKCFEGFGIDGPAIAKCLGEKWSHPPSCIKTDCLSLPSFENAIPMGEKKDVYKAGEQVTYTCATYYKMDGASNVTCINSRWTGRPTCRDTSCVNPPTVQNAYIVSRQMSKYPSGERVRYQCRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKR

SEQ ID NO: 7 - protein, 1213 aa, Homo sapiens
EDCNELPPRRNTEILTGSWSDQTYPEGTQAIYKCRPGYRSLGNIIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNHGRKFVQGKSIDVACHPGYALPKAQTTVTCMENGWSPTPRCIRVKTCSKSSIDIENGFISESQYTYALKEKAKYQCKLGYVTADGETSGSITCGKDGWSAQPTCIKSCDIPVFMNARTKNDFTWFKLNDTLDYECHDGYESNTGSTTGSIVCGYNGWSDLPICYERECELPKIDVHLVPDRKKDQYKVGEVLKFSCKPGFTIVGPNSVQCYHFGLSPDLPICKEQVQSCGPPPELLNGNVKEKTKEEYGHSEVVEYYCNPRFLMKGPNKIQCVDGEWTTLPVCIVEESTCGDIPELEHGWAQLSSPPYYYGDSVEFNCSESFTMIGHRSITCIHGVWTQLPQCVAIDKLKKCKSSNLIILEEHLKNKKEFDHNSNIRYRCRGKEGWIHTVCINGRWDPEVNCSMAQIQLCPPPPQIPNSHNMTTTLNYRDGEKVSVLCQENYLIQEGEEITCKDGRWQSIPLCVEKIPCSQPPQIEHGTINSSRSSQESYAHGTKLSYTCEGGFRISEENETTCYMGKWSSPPQCEGLPCKSPPEISHGVVAHMSDSYQYGEEVTYKCFEGFGIDGPAIAKCLGEKWSHPPSCIKTDCLSLPSFENAIPMGEKKDVYKAGEQVTYTCATYYKMDGASNVTCINSRWTGRPTCRDTSCVNPPTVQNAYIVSRQMSKYPSGERVRYQCRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKR

SEQ ID NO: 8 - protein, 1213 aa, Homo sapiens
EDCNELPPRRNTEILTGSWSDQTYPEGTQAIYKCRPGYRSLGNIIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNYGRKFVQGKSIDVACHPGYALPKAQTTVTCMENGWSPTPRCIRVKTCSKSSIDIENGFISESQYTYALKEKAKYQCKLGYVTADGETSGSITCGKDGWSAQPTCIKSCDIPVFMNARTKNDFTWFKLNDTLDYECHDGYESNTGSTTGSIVCGYNGWSDLPICYERECELPKIDVHLVPDRKKDQYKVGEVLKFSCKPGFTIVGPNSVQCYHFGLSPDLPICKEQVQSCGPPPELLNGNVKEKTKEEYGHSEVVEYYCNPRFLMKGPNKIQCVDGEWTTLPVCIVEESTCGDIPELEHGWAQLSSPPYYYGDSVEFNCSESFTMIGHRSITCIHGVWTQLPQCVAIDKLKKCKSSNLIILEEHLKNKKEFDHNSNIRYRCRGKEGWIHTVCINGRWDPEVNCSMAQIQLCPPPPQIPNSHNMTTTLNYRDGEKVSVLCQENYLIQEGEEITCKDGRWQSIPLCVEKIPCSQPPQIEHGTINSSRSSQESYAHGTKLSYTCEGGFRISEENETTCYMGKWSSPPQCEGLPCKSPPDISHGVVAHMSDSYQYGEEVTYKCFEGFGIDGPAIAKCLGEKWSHPPSCIKTDCLSLPSFENAIPMGEKKDVYKAGEQVTYTCATYYKMDGASNVTCINSRWTGRPTCRDTSCVNPPTVQNAYIVSRQMSKYPSGERVRYQCRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKR

SEQ ID NO: 9 - protein, 1213 aa, Homo sapiens
EDCNELPPRRNTEILTGSWSDQTYPEGTQAIYKCRPGYRSLGNIIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNHGRKFVQGKSIDVACHPGYALPKAQTTVTCMENGWSPTPRCIRVKTCSKSSIDIENGFISESQYTYALKEKAKYQCKLGYVTADGETSGSITCGKDGWSAQPTCIKSCDIPVFMNARTKNDFTWFKLNDTLDYECHDGYESNTGSTTGSIVCGYNGWSDLPICYERECELPKIDVHLVPDRKKDQYKVGEVLKFSCKPGFTIVGPNSVQCYHFGLSPDLPICKEQVQSCGPPPELLNGNVKEKTKEEYGHSEVVEYYCNPRFLMKGPNKIQCVDGEWTTLPVCIVEESTCGDIPELEHGWAQLSSPPYYYGDSVEFNCSESFTMIGHRSITCIHGVWTQLPQCVAIDKLKKCKSSNLIILEEHLKNKKEFDHNSNIRYRCRGKEGWIHTVCINGRWDPEVNCSMAQIQLCPPPPQIPNSHNMTTTLNYRDGEKVSVLCQENYLIQEGEEITCKDGRWQSIPLCVEKIPCSQPPQIEHGTINSSRSSQESYAHGTKLSYTCEGGFRISEENETTCYMGKWSSPPQCEGLPCKSPPDISHGVVAHMSDSYQYGEEVTYKCFEGFGIDGPAIAKCLGEKWSHPPSCIKTDCLSLPSFENAIPMGEKKDVYKAGEQVTYTCATYYKMDGASNVTCINSRWTGRPTCRDTSCVNPPTVQNAYIVSRQMSKYPSGERVRYQCRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKR

SEQ ID NO: 10 - protein, 431 aa, Homo sapiens
EDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNYGRKFVQGKSIDVACHPGYALPKAQTTVTCMENGWSPTPRCIRVSFTL

(19) United States
(12) Patent Application Publication
(10) Pub. No.: US 2024/0024416 A1
(43) Pub. Date: Jan. 25, 2024

(54) METHODS FOR TREATING INFLAMMATORY OCULAR DISEASES WITH COMPLEMENT FACTOR H

(71) Applicant: Gemini Therapeutics Sub, Inc., Cambridge, MA (US)

(72) Inventors: Suresh Katti, Hamden, CT (US); Scott Lauder, Somerville, MA (US); Robyn Biggs, Arlington, MA (US); Walter Strapps, Dedham, MA (US); Lenore von Krusenstiern, Brookline, MA (US)

(21) Appl. No.: 18/251,181

(22) PCT Filed: Oct. 29, 2021
(86) PCT No.: PCT/US2021/057449

§ 371 Date: Apr. 28, 2023

Related U.S. Application Data
(60) Provisional application No. 63/107,736, filed on Oct. 30, 2020.

(51) Int. Cl. A61K 38/17; A61P 27/02; C07K 16/22; C12N 15/86

(52) U.S. Cl. CPC A61K 38/1725; A61P 27/02; C07K 16/22; C12N 15/86

(57) ABSTRACT

The present disclosure relates to dosage regimens of complement factor H (CFH) protein for treating patients having inflammatory ocular diseases or conditions (e.g., age-related macular degeneration or early-onset macular dystrophies). The present disclosure also provides methods of treating patients having inflammatory ocular diseases, disorders, or conditions (e.g., age-related macular degeneration) using complement factor H (CFH) based on the levels of certain protein biomarkers, such as complement components and inflammation markers, in ocular samples of the patients.

### CROSS-REFERENCE TO RELATED APPLICATION

This application claims the benefit of and priority to U.S. Provisional Patent Application No. 63/107,736, filed Oct. 30, 2020, the entire disclosure of which is hereby incorporated by reference herein in its entirety for all purposes.

### SEQUENCE LISTING

The instant application contains a Sequence Listing which has been submitted electronically in ASCII format and is hereby incorporated by reference in its entirety. Said ASCII copy, created on Oct. 26, 2021, is named GEM-019WO_SL_ST25.txt and is 98,500 bytes in size.

### FIELD OF THE INVENTION

The invention relates to dosage regimens of complement factor H (CFH) protein for treating patients having inflammatory ocular diseases or conditions (e.g., age-related macular degeneration or early-onset macular dystrophies). The invention also relates to methods of treating patients having inflammatory ocular diseases or conditions using CFH based on the levels of certain protein biomarkers in ocular samples of the patients.

### BACKGROUND OF THE DISCLOSURE

Age-related macular degeneration (AMD) is a medical condition and is the leading cause of legal blindness in Western societies. AMD typically affects older adults and results in a loss of central vision due to degenerative and neovascular changes to the macula, a pigmented region at the center of the retina which is responsible for visual acuity. There are four major AMD subtypes: early AMD; intermediate AMD; advanced non-neovascular AMD (“dry AMD”); and advanced neovascular AMD (“wet AMD”). Typically, AMD is identified by the focal hyperpigmentation of the retinal pigment epithelium (RPE) and accumulation of drusen deposits and/or geographic atrophy (GA). The size and number of drusen deposits and level of geographic atrophy typically correlates with AMD severity.

AMD occurs in up to 8% of individuals over the age of 60, and the prevalence of AMD continues to increase with age. The U.S. is anticipated to have nearly 22 million cases of AMD by the year 2050, while global cases of AMD are expected to be nearly 288 million by the year 2040. There is a need for novel treatments for preventing progression from early to intermediate and/or from intermediate to advanced stages of AMD to prevent loss of vision. In particular, there remains a need to identify patients that are likely to respond to such novel treatments and a need to monitor the response and efficacy of patients to such novel treatments.

### SUMMARY OF THE DISCLOSURE

The disclosure relates to dosage regimens of complement factor H (CFH) protein for treating patients having inflammatory ocular diseases or conditions (e.g., age-related macular degeneration or early-onset macular dystrophies). The present disclosure also provides methods for treating, preventing, or inhibiting an inflammatory ocular disease, disorder, or condition comprising administering to a subject complement factor H (CFH).

Accordingly, in one aspect, the present disclosure provides a method of treating a subject having an inflammatory ocular disease, disorder, or condition, the method comprising administering to the subject 50 μg, 100 μg, 250 μg, or 500 μg of CFH protein per eye, thereby treating the inflammatory ocular disease, disorder, or condition. In certain embodiments, the CFH protein is administered to the subject by intravitreal (IVT) injection.

In certain embodiments, the inflammatory ocular disease, disorder, or condition is selected from the group consisting of macular degeneration, diabetic retinopathy, diabetic macular edema, retinal vein occlusion, uveitis, sterile conjunctivitis, keratitis, episcleritis, and Stargardt's Disease. In certain embodiments, the macular degeneration is AMD. In certain embodiments, the AMD is dry AMD. In certain embodiments, the inflammatory ocular disease, disorder, or condition comprises geographic atrophy secondary to dry AMD. In certain embodiments, the AMD is neovascular AMD. In certain embodiments, the subject has been treated with a VEGF-A antagonist. In certain embodiments, the uveitis is anterior uveitis.

In certain embodiments, the CFH is administered by intravitreal injection.

In certain embodiments, the CFH protein comprises a recombinant CFH protein. In certain embodiments, the CFH protein comprises the amino acid sequence of SEQ ID NO: 2.

In certain embodiments, the CFH protein is administered (e.g., intravitreally) at a dose of 50 μg per eye. In certain embodiments, the CFH protein is administered (e.g., intravitreally) at a dose of 100 μg per eye. In certain embodiments, the CFH protein is administered (e.g., intravitreally) at a dose of 250 μg per eye. In certain embodiments, the CFH protein is administered (e.g., intravitreally) at a dose of 500 μg per eye. In certain embodiments, the CFH protein is administered once every four weeks, once every eight weeks, once every month, once every two months, once every three months, once every four months, once every five months, or once every six months. In certain embodiments, the dose of CFH protein is 250 μg per eye, administered once every month or once every four weeks. In certain embodiments, the dose of CFH protein is 250 μg per eye, administered once every two months or once every eight weeks. In certain embodiments, the dose of CFH protein is 500 μg per eye, administered once every month or once every four weeks. In certain embodiments, the dose of CFH protein is 500 μg per eye, administered once every two months or once every eight weeks.

In certain embodiments, a lesion associated with the inflammatory ocular disease, disorder, or condition is observed in the subject, for example, by fundus imaging. In certain embodiments, the subject carries a genetic variant associated with the inflammatory ocular disease, disorder, or condition. In certain embodiments, the inflammatory ocular disease, disorder, or condition is geographic atrophy, and wherein the genetic variant results in a mutation of the Tyr at amino acid position 402 of human CFH protein and is present in both alleles of the genome. In certain embodiments, the genetic variant results in a mutation of the Tyr at amino acid position 402 of human CFH protein to His residue and is present in both alleles of the genome. In certain embodiments, the genetic variant comprises rs1061170 in both alleles of the human genome. In certain embodiments, the subject is homozygous for the Y402H mutation of the CFH gene. In certain embodiments, the subject is negative for any one of the missense CFH mutations selected from the group consisting of R2T, L3V, R53C, R53H, S58A, D90G, D130N, R175Q, R175P, I221V, R303W, R303Q, Q400K, P503A, R567G, G650V, S890I, T956M, G1194D, R1210C, and any combination(s) thereof. In certain embodiments, the subject is negative for a combination of homozygous CFH 62V, homozygous C3 102G, and homozygous CFB 32R. In certain embodiments, the subject is (a) homozygous for the Y402H mutation of CFH; (b) negative for any one of the missense CFH mutations selected from the group consisting of R2T, L3V, R53C, R53H, S58A, D90G, D130N, R175Q, R175P, I221V, R303W, R303Q, Q400K, P503A, R567G, G650V, S890I, T956M, G1194D, R1210C, and any combination(s) thereof; and (c) negative for a combination of homozygous CFH 62V, homozygous C3 102G, and homozygous CFB 32R. In certain embodiments, the subject is positive for a missense CFH mutation selected from the group consisting of R2T, L3V, R53C, R53H, S58A, D90G, D130N, R175Q, R175P, I221V, R303W, R303Q, Q400K, P503A, R567G, G650V, S890I, T956M, G1194D, R1210C, and any combination(s) thereof. In certain embodiments, the subject is positive for a combination of homozygous CFH 62V, homozygous C3 102G, and homozygous CFB 32R. In certain embodiments, the subject is at least 50 years old.

In certain embodiments, the method further comprises administering to the subject an effective amount of a VEGF antagonist. In certain embodiments, the subject has neovascular AMD. In certain embodiments, the VEGF antagonist comprises aflibercept. In certain embodiments, the effective amount of aflibercept is 2 mg per eye. In certain embodiments, the aflibercept is administered once every four weeks, once every eight weeks, once every month, or once every two months.

In certain embodiments, the treatment slows the progression of the inflammatory ocular disease, disorder, or condition in the subject. In certain embodiments, the treatment reduces the severity of the inflammatory ocular disease, disorder, or condition in the subject. In certain embodiments, the progression or severity of the inflammatory ocular disease, disorder, or condition is assessed by one or more methods selected from:
(a) best corrected visual acuity (BCVA) score;
(b) low luminance visual acuity (LLVA) score;
(c) AREDS 9-step severity scale score;
(d) area of geographic atrophy assessed by color fundus photography, fundus autofluorescence, optical coherence tomography, optical coherence tomography—angiography, near infrared imaging, and/or fluorescein angiography;
(e) drusen volume; and
(f) one or more retinal architecture parameters assessed by optical coherence tomography, selected from total retinal and choroidal thickness, photoreceptor layer thickness, features of nascent geographic atrophy, retinal pigment epithelium thickening, and integrity of retinal pigment epithelium layer.

Accordingly, in another aspect, the present disclosure provides a method for treating a subject having an inflammatory ocular disease, disorder, or condition, the method comprising:
(a) obtaining or having obtained a measurement of the protein level of at least one biomarker in an ocular sample of the subject, wherein the biomarker is selected from the group consisting of (i) complement component C3, complement factor B (CFB), complement component C5, cleavage fragments thereof, and complement factor H (CFH), (ii) proteins associated with ocular inflammation, and (iii) proteins associated with choroidal neovascularization;
(b) determining whether the protein level of the biomarker is greater than or lower than a predetermined threshold; and
(c) administering to the subject an effective amount of CFH if the protein level of the biomarker is (i) greater than or equal to the threshold if the biomarker is positively correlated with activation of the complement pathway or with inflammation or (ii) lower than or equal to the threshold if the biomarker is negatively correlated with activation of the complement pathway or with inflammation,
thereby treating the inflammatory ocular disease, disorder, or condition.

In certain embodiments, the inflammatory ocular disease, disorder, or condition is selected from the group consisting of macular degeneration, diabetic retinopathy, diabetic macular edema, retinal vein occlusion, uveitis, sterile conjunctivitis, keratitis, episcleritis, and Stargardt's Disease. In certain embodiments, the macular degeneration is age-related macular degeneration (AMD). In certain embodiments, the AMD is dry AMD. In certain embodiments, the inflammatory ocular disease, disorder, or condition comprises geographic atrophy secondary to dry AMD. In certain embodiments, the AMD is neovascular AMD. In certain embodiments, the subject has been treated with a VEGF (e.g., VEGF-A) antagonist. In certain embodiments, the uveitis is anterior uveitis.

In certain embodiments, the ocular sample comprises aqueous humor.

In certain embodiments, the at least one biomarker comprises a cleavage fragment of C3, CFB, or C5, and the protein level of the cleavage fragment is positively correlated with activation of the complement pathway. In certain embodiments, the cleavage fragment is selected from the group consisting of C3a, Ba, and C5a. In certain embodiments, the cleavage fragment is C3a. In certain embodiments, the threshold of C3a is 2 ng/mL. In certain embodiments, the cleavage fragment is Ba. In certain embodiments, the threshold of Ba is 8 ng/mL. In certain embodiments, the at least one biomarker comprises CFH, and the protein level of CFH is negatively correlated with activation of the complement pathway. In certain embodiments, the threshold of CFH is 60 ng/mL.

In certain embodiments, the at least one biomarker comprises a protein associated with ocular inflammation. In certain embodiments, the protein associated with ocular inflammation is selected from the group consisting of IL-1β, IL-6, IL-8, IL-10, IL-18, TNF-α, CCL2, CXCL5, and Eotaxin-2, and the protein level of the biomarker is positively correlated with ocular inflammation.

In certain embodiments, the at least one biomarker comprises a protein associated with choroidal neovascularization. In certain embodiments, the protein associated with choroidal neovascularization is VEGF-A, and the protein level of the biomarker is positively correlated with choroidal neovascularization.

In certain embodiments, the CFH is administered by intravitreal injection.

In certain embodiments, the CFH comprises a CFH protein. In certain embodiments, the CFH protein comprises a recombinant CFH protein. In certain embodiments, the CFH protein is administered at a dose of 50 μg, 100 μg, 250 μg, or 500 μg per eye. In certain embodiments, the CFH protein is administered (e.g., intravitreally) at a dose of 50 μg per eye. In certain embodiments, the CFH protein is administered (e.g., intravitreally) at a dose of 100 μg per eye. In certain embodiments, the CFH protein is administered (e.g., intravitreally) at a dose of 250 μg per eye. In certain embodiments, the CFH protein is administered (e.g., intravitreally) at a dose of 500 μg per eye. In certain embodiments, the CFH protein is administered once every four weeks, once every eight weeks, once every month, once every two months, once every three months, once every four months, once every five months, or once every six months. In certain embodiments, the dose of CFH protein is 250 μg per eye, administered once every month or once every four weeks. In certain embodiments, the dose of CFH protein is 250 μg per eye, administered once every two months or once every eight weeks. In certain embodiments, the dose of CFH protein is 500 μg per eye, administered once every month or once every four weeks. In certain embodiments, the dose of CFH protein is 500 μg per eye, administered once every two months or once every eight weeks.

In certain embodiments, the CFH comprises a vector encoding a CFH protein. In certain embodiments, the vector is an adeno-associated virus vector.

In certain embodiments, the CFH protein comprises the amino acid sequence of SEQ ID NO: 2.

In certain embodiments, the steps (a)-(c) are conducted after an initial administration of CFH to the subject.

In certain embodiments, a lesion associated with the inflammatory ocular disease, disorder, or condition is observed in the subject, for example, by fundus imaging. In certain embodiments, the subject carries a genetic variant associated with the inflammatory ocular disease, disorder, or condition. In certain embodiments, the inflammatory ocular disease, disorder, or condition is geographic atrophy, and wherein the genetic variant results in a mutation of the Tyr at amino acid position 402 of human CFH protein and is present in both alleles of the genome. In certain embodiments, the genetic variant results in a mutation of the Tyr at amino acid position 402 of human CFH protein to His residue and is present in both alleles of the genome. In certain embodiments, the genetic variant comprises rs1061170 in both alleles of the human genome. In certain embodiments, the subject is homozygous for the Y402H mutation of the CFH gene. In certain embodiments, the subject is negative for any one of the missense CFH mutations selected from the group consisting of R2T, L3V, R53C, R53H, S58A, D90G, D130N, R175Q, R175P, I221V, R303W, R303Q, Q400K, P503A, R567G, G650V, S890I, T956M, G1194D, R1210C, and any combination(s) thereof. In certain embodiments, the subject is negative for a combination of homozygous CFH 62V, homozygous C3 102G, and homozygous CFB 32R. In certain embodiments, the subject is (a) homozygous for the Y402H mutation of CFH; (b) negative for any one of the missense CFH mutations selected from the group consisting of R2T, L3V, R53C, R53H, S58A, D90G, D130N, R175Q, R175P, I221V, R303W, R303Q, Q400K, P503A, R567G, G650V, S890I, T956M, G1194D, R1210C, and any combination(s) thereof; and (c) negative for a combination of homozygous CFH 62V, homozygous C3 102G, and homozygous CFB 32R. In certain embodiments, the subject is positive for a missense CFH mutation selected from the group consisting of R2T, L3V, R53C, R53H, S58A, D90G, D130N, R175Q, R175P, I221V, R303W, R303Q, Q400K, P503A, R567G, G650V, S890I, T956M, G1194D, R1210C, and any combination(s) thereof. In certain embodiments, the subject is positive for a combination of homozygous CFH 62V, homozygous C3 102G, and homozygous CFB 32R. In certain embodiments, the subject is at least 50 years old.

In certain embodiments, the method further comprises administering to the subject an effective amount of a VEGF antagonist. In certain embodiments, the subject has neovascular AMD. In certain embodiments, the VEGF antagonist comprises aflibercept. In certain embodiments, the effective amount of aflibercept is 2 mg per eye. In certain embodiments, the aflibercept is administered once every four weeks, once every eight weeks, once every month, or once every two months.

In certain embodiments, the treatment slows the progression of the inflammatory ocular disease, disorder, or condition in the subject. In certain embodiments, the treatment reduces the severity of the inflammatory ocular disease, disorder, or condition in the subject. In certain embodiments, the progression or severity of the inflammatory ocular disease, disorder, or condition is assessed by one or more methods selected from:
(a) best corrected visual acuity (BCVA) score;
(b) low luminance visual acuity (LLVA) score;
(c) AREDS 9-step severity scale score;
(d) area of geographic atrophy assessed by color fundus photography, fundus autofluorescence, optical coherence tomography, optical coherence tomography—angiography, near infrared imaging, and/or fluorescein angiography;
(e) drusen volume; and
(f) one or more retinal architecture parameters assessed by optical coherence tomography, selected from total retinal and choroidal thickness, photoreceptor layer thickness, features of nascent geographic atrophy, retinal pigment epithelium thickening, and integrity of retinal pigment epithelium layer.

In another aspect, the present disclosure provides a method for selecting a subject for treatment of an inflammatory ocular disease, disorder, or condition, the method comprising:
(a) obtaining or having obtained a measurement of the protein level of at least one biomarker in an ocular sample of the subject, wherein the biomarker is selected from the group consisting of (i) complement component C3, CFB, complement component C5, cleavage fragments thereof, and CFH, (ii) proteins associated with ocular inflammation, and (iii) proteins associated with choroidal neovascularization;
(b) determining whether the protein level of the biomarker is greater than or lower than a predetermined threshold; and
(c) selecting the subject for treatment of the inflammatory ocular disease, disorder, or condition if the protein level of the biomarker is (i) greater than or equal to the threshold if the biomarker is positively correlated with activation of the complement pathway, ocular inflammation, or choroidal neovascularization; or (ii) lower than or equal to the threshold if the biomarker is negatively correlated with activation of the complement pathway, ocular inflammation, or choroidal neovascularization,
wherein the treatment comprises administering to the subject CFH.

In certain embodiments, step (a) comprises measuring the protein level of the biomarker in an ocular sample of the subject.

In certain embodiments, the inflammatory ocular disease, disorder, or condition is selected from the group consisting of macular degeneration, diabetic retinopathy, diabetic macular edema, retinal vein occlusion, uveitis, sterile conjunctivitis, keratitis, episcleritis, and Stargardt's Disease. In certain embodiments, the macular degeneration is AMD. In certain embodiments, the AMD is dry AMD. In certain embodiments, the inflammatory ocular disease, disorder, or condition comprises geographic atrophy secondary to dry AMD. In certain embodiments, the AMD is neovascular AMD. In certain embodiments, the subject has been treated with a VEGF (e.g., VEGF-A) antagonist. In certain embodiments, the uveitis is anterior uveitis.

In certain embodiments, the ocular sample comprises aqueous humor.

In certain embodiments, the at least one biomarker comprises a cleavage fragment of C3, CFB, or C5, and the protein level of the cleavage fragment is positively correlated with activation of the complement pathway. In certain embodiments, the biomarker is selected from the group consisting of C3a, Ba, and C5a. In certain embodiments, the biomarker is C3a. In certain embodiments, the threshold of C3a is 2 ng/mL. In certain embodiments, the biomarker is Ba. In certain embodiments, the threshold of Ba is 8 ng/mL. In certain embodiments, the at least one biomarker comprises CFH, and the protein level of CFH is negatively correlated with activation of the complement pathway. In certain embodiments, the threshold of CFH is 60 ng/mL.

In certain embodiments, the at least one biomarker comprises a protein associated with ocular inflammation. In certain embodiments, the protein associated with ocular inflammation is selected from the group consisting of IL-1β, IL-6, IL-8, IL-10, IL-18, TNF-α, CCL2, CXCL5, and Eotaxin-2, and the protein level of the biomarker is positively correlated with ocular inflammation.

In certain embodiments, the at least one biomarker comprises a protein associated with choroidal neovascularization. In certain embodiments, the protein associated with choroidal neovascularization is VEGF-A, and the protein level of the biomarker is positively correlated with choroidal neovascularization.

In certain embodiments, the CFH is administered by intravitreal injection.

In certain embodiments, the CFH comprises a CFH protein. In certain embodiments, the CFH protein comprises a recombinant CFH protein. In certain embodiments, the CFH protein is administered at a dose of 50 μg, 100 μg, 250 μg, or 500 μg per eye. In certain embodiments, the CFH protein is administered (e.g., intravitreally) at a dose of 50 μg per eye. In certain embodiments, the CFH protein is administered (e.g., intravitreally) at a dose of 100 μg per eye. In certain embodiments, the CFH protein is administered (e.g., intravitreally) at a dose of 250 μg per eye. In certain embodiments, the CFH protein is administered (e.g., intravitreally) at a dose of 500 μg per eye. In certain embodiments, the CFH protein is administered once every four weeks, once every eight weeks, once every month, once every two months, once every three months, once every four months, once every five months, or once every six months. In certain embodiments, the dose of CFH protein is 250 μg per eye, administered once every month or once every four weeks. In certain embodiments, the dose of CFH protein is 250 μg per eye, administered once every two months or once every eight weeks. In certain embodiments, the dose of CFH protein is 500 μg per eye, administered once every month or once every four weeks. In certain embodiments, the dose of CFH protein is 500 μg per eye, administered once every two months or once every eight weeks.

In certain embodiments, the CFH comprises a vector encoding a CFH protein. In certain embodiments, the vector is an adeno-associated virus vector.

In certain embodiments, the CFH protein comprises the amino acid sequence of SEQ ID NO: 2.

In certain embodiments, the steps (a)-(c) are conducted after an initial administration of CFH to the subject.

In certain embodiments, a lesion associated with the inflammatory ocular disease, disorder, or condition is observed in the subject, for example, by fundus imaging. In certain embodiments, the subject carries a genetic variant associated with the inflammatory ocular disease, disorder, or condition. In certain embodiments, the inflammatory ocular disease, disorder, or condition is geographic atrophy, and wherein the genetic variant results in a mutation of the Tyr at amino acid position 402 of human CFH protein and is present in both alleles of the genome. In certain embodiments, the genetic variant comprises rs1061170 in both alleles of the human genome. In certain embodiments, the genetic variant results in a mutation of the Tyr at amino acid position 402 of human CFH protein to His residue and is present in both alleles of the genome. In certain embodiments, the genetic variant comprises rs1061170 in both alleles of the human genome. In certain embodiments, the subject is homozygous for the Y402H mutation of the CFH gene. In certain embodiments, the subject is negative for any one of the missense CFH mutations selected from the group consisting of R2T, L3V, R53C, R53H, S58A, D90G, D130N, R175Q, R175P, I221V, R303W, R303Q, Q400K, P503A, R567G, G650V, S890I, T956M, G1194D, R1210C, and any combination(s) thereof. In certain embodiments, the subject is negative for a combination of homozygous CFH 62V, homozygous C3 102G, and homozygous CFB 32R. In certain embodiments, the subject is (a) homozygous for the Y402H mutation of CFH; (b) negative for any one of the missense CFH mutations selected from the group consisting of R2T, L3V, R53C, R53H, S58A, D90G, D130N, R175Q, R175P, I221V, R303W, R303Q, Q400K, P503A, R567G, G650V, S890I, T956M, G1194D, R1210C, and any combination(s) thereof; and (c) negative for a combination of homozygous CFH 62V, homozygous C3 102G, and homozygous CFB 32R. In certain embodiments, the subject is positive for a missense CFH mutation selected from the group consisting of R2T, L3V, R53C, R53H, S58A, D90G, D130N, R175Q, R175P, I221V, R303W, R303Q, Q400K, P503A, R567G, G650V, S890I, T956M, G1194D, R1210C, and any combination(s) thereof. In certain embodiments, the subject is positive for a combination of homozygous CFH 62V, homozygous C3 102G, and homozygous CFB 32R. In certain embodiments, the subject is at least 50 years old.

In certain embodiments, the method further comprises administering to the subject an effective amount of a VEGF antagonist. In certain embodiments, the subject has neovascular AMD. In certain embodiments, the VEGF antagonist comprises aflibercept. In certain embodiments, the effective amount of aflibercept is 2 mg per eye. In certain embodiments, the aflibercept is administered once every four weeks, once every eight weeks, once every month, or once every two months.

In certain embodiments, the treatment slows the progression of the inflammatory ocular disease, disorder, or condition in the subject. In certain embodiments, the treatment reduces the severity of the inflammatory ocular disease, disorder, or condition in the subject. In certain embodiments, the progression or severity of the inflammatory ocular disease, disorder, or condition is assessed by one or more methods selected from:
(a) best corrected visual acuity (BCVA) score;
(b) low luminance visual acuity (LLVA) score;
(c) AREDS 9-step severity scale score;
(d) area of geographic atrophy assessed by color fundus photography, fundus autofluorescence, optical coherence tomography, optical coherence tomography—angiography, near infrared imaging, and/or fluorescein angiography;
(e) drusen volume; and
(f) one or more retinal architecture parameters assessed by optical coherence tomography, selected from total retinal and choroidal thickness, photoreceptor layer thickness, features of nascent geographic atrophy, retinal pigment epithelium thickening, and integrity of retinal pigment epithelium layer.

In another aspect.

### BRIEF DESCRIPTION OF THE DRAWINGS

FIGS. 1A-1C are a set of graphs showing reduction of C3bBb deposition (FIG. 1A), reduction of C3a generation (FIG. 1B), and reduction of Ba generation (FIG. 1C) in a retinal pigment epithelium (RPE) cell culture as a result of exogenous supplementation of recombinant CFH protein (rCFH) in a dose dependent manner.

FIG. 2 is a diagram of a clinical study design for a single-ascending dose (SAD) of recombinant CFH illustrating a cohort patient flow. DLTs=dose limiting toxicity; MTD=maximum tolerated dose; n=number of subjects; TBD=to be determined; wk=week.

FIGS. 3A-3B are a set of graphs showing the levels of Ba (FIG. 3A) and C3a (FIG. 3B) in aqueous humor samples obtained from patients on the days indicated after the rCFH treatment relative to the respective levels prior to the rCFH treatment (Day 0). The p values were calculated under the two tailed t-test, which makes no assumption of expected direction of change. Where an assumption of reduced Ba and C3a levels is made, the p values should be reduced by half.

FIGS. 4A-4B are a set of graphs showing the best corrected visual acuity (BCVA) (FIG. 4A) and low luminance visual acuity (LLVA) (FIG. 4B) scores of the patients enrolled in the phase 1 clinical study on the days indicated after the rCFH treatment relative to the respective scores prior to the rCFH treatment (Day 0).

FIG. 5 is a diagram of a clinical study design for a Phase 2 multiple dose study of recombinant CFH (rCFH) in patients with dry AMD. BL=baseline; min=minimum; PD=pharmacodynamics; PK=pharmacokinetics; q30d=every 30 days; q60d=every 60 days; *In Version 1.0 of the protocol, eligible subjects with non-central GA were enrolled in Cohort 1 and those with central GA were enrolled in Cohort 2.

FIG. 6 is a graph showing total CFH levels in aqueous humor samples obtained from patients on the days indicated after the rCFH treatment relative to the respective levels prior to the rCFH treatment (Day 0). By the indicated time points, the patients were administered either 250 μg of rCFH monthly or 500 μg of rCFH monthly.

FIGS. 7A-7C are a set of graphs showing Ba concentration levels in aqueous humor after administration of either 250 μg of rCFH monthly or 500 μg of rCFH monthly (FIG. 7A); 250 μg of rCFH monthly (FIG. 7B); or 500 μg of rCFH monthly(FIG. 7C). “Healthy” Ba levels in FIGS. 7A-7C range from 7 ng/mL to 21 ng/mL (with an average of 11.7 ng/mL). Quartiles 1-4 (abbreviated as Q1-Q4) denote Ba levels between 5.4 ng/mL and 16.65 ng/mL; between 16.91 ng/mL and 25.87 ng/mL; between 26.25 ng/mL and 44.22 ng/mL; and between 44.46 ng/mL and 165.6 ng/mL, respectively.

FIG. 8 is a graph showing C3a concentration levels in aqueous humor after administration of either 250 μg of rCFH monthly or 500 μg of rCFH monthly. “Healthy” C3a levels in range from 1300 μg/mL to 4100 μg/mL (with an average of 2700 μg/mL). Quartiles 1-4 denote C3a levels between 1097 μg/mL and 3442 μg/mL; between 3510 μg/mL and 4950 μg/mL; between 5040 μg/mL and 6968 μg/mL; and between 7022 μg/mL and 17882 μg/mL, respectively.

FIG. 9 is a scatter dot plot showing correlation Ba and C3a concentration levels at baseline and after administration of rCFH.

FIG. 10 is a diagram of a clinical study design for Phase 2 multiple dose study of recombinant CFH (rCFH) as an adjunct to standard of care Aflibercept therapy in patients with neovascular AMD (nAMD).

### DETAILED DESCRIPTION OF THE DISCLOSURE

The disclosure provides methods for treating, preventing, or inhibiting an inflammatory ocular disease, disorder, or condition comprising administering to a subject complement factor H (CFH). In one aspect, the disclosure provides dosage regimens of CFH protein for treating an ocular disease or disorder. In another aspect, the disclosure provides a method of treating, preventing, or inhibiting an inflammatory ocular disease, disorder, or condition in a patient who is selected based on the protein level of one or more biomarkers in an ocular sample, relative to a predetermined threshold, and intraocularly (e.g., intravitreally) administering an effective amount of CFH (e.g., a recombinant CFH protein or biologically active fragments and/or variants thereof).

A wide variety of diseases of the eye associated with the presence of selected biomarkers may be treated or prevented using the methods provided herein. Inflammatory ocular diseases, disorders, or conditions that may be treated or prevented using the methods of the disclosure include but are not limited to glaucoma diabetic retinopathies, inherited retinal degeneration such as retinitis pigmentosa, retinal detachment or injury and retinopathies (such as retinopathies that are inherited, induced by surgery, trauma, an underlying aetiology such as severe anemia, SLE, hypertension, blood dyscrasias, systemic infections, or underlying carotid disease, a toxic compound or agent, or photically). Inflammatory ocular diseases, disorders, or conditions include, but is not limited to, macular degeneration, e.g., age-related macular degeneration (AMD), dry AMD, geographic atrophy (GA) secondary to dry AMD, neovascular AMD, uveitis (e.g., anterior uveitis), sterile conjunctivitis, keratitis, episcleritis, Stargardt's Disease, diabetic retinopathy, diabetic macular edema, and retinal vein occlusion.

### Definitions

The following terms, unless otherwise indicated, shall be understood to have the following meanings:

As used herein, and unless otherwise stated, the term “complement factor H” or “CFH” encompasses all the isoforms of complement factor H, including the canonical isoform (the human protein identified as P08603-1 in UniProt) and a second isoform called factor-H-like protein 1 (FHL1) (the human protein identified as P08603-2 in UniProt), and functional fragments and/or variants thereof. Exemplary amino acid sequences of CFH are provided in SEQ ID NOs: 1-10. Unless indicated otherwise expressly or by context (e.g., “recombinant CFH” or “rCFH”), this term also refers to a vector encoding a CFH protein.

As used herein, the term “biomarker” refers to a substance, e.g., a protein or a nucleic acid, where the presence, absence or concentration of the biomarker in a biological sample from a subject indicates the subject's disease status and/or potential response or lack of response to a therapeutic treatment. For example, the concentration of a biomarker in an ocular or blood sample from a subject having an inflammatory ocular disease, disorder, or condition, relative to the concentration of the biomarker in a control sample of the same type (e.g., a sample of the same type from a subject not having the inflammatory ocular disease, disorder, or condition), may be used to select a subject for a given therapeutic treatment and/or to monitor the progress of the treatment.

As used herein, the term “cleavage fragment” refers to a peptide fragment of a larger precursor protein, wherein the cleavage fragment is produced as a consequence of a proteolytic cleavage event, e.g., cleavage by a protease or self-cleavage.

As used herein, the term “proteins associated with ocular inflammation” refers to any protein that is associated with increased inflammation, either with inducing inflammation or as a result of inflammatory responses in an ocular tissue. Examples of proteins associated with inflammation include, but are not limited to inflammatory cytokines (e.g., interleukin-1 (IL-1), interleukin-1 beta (IL-1), interleukin-6 (IL-6), interleukin-8 (IL-8), interleukin-10 (IL-10), interleukin 12 (IL-12), interleukin 18 (IL-18), tumor necrosis factor alpha (TNFα, a.k.a. TNF), interferon gamma (INF-γ), granulocyte-macrophage colony stimulating factor (GMCSF)), chemokines (e.g., (C-Cmotif) ligand 2 (CCL2)/monocyte chemoattractant protein 1 (MCP1), C-X-C motif chemokine 5 (CXCL5), C-C motif chemokine 24 (CCL24, a.k.a. Eotaxin-2)), and other factors (e.g., uncleaved complement component C3).

As used herein, the terms “patient,” “subject,” and “individual” are used interchangeably herein and refer to either a human or a non-human animal. These terms include mammals, such as humans, non-human primates, laboratory animals, livestock animals (including bovines, porcines, camels, etc.), companion animals (e.g., canines, felines, other domesticated animals, etc.) and rodents (e.g., mice and rats).

As used herein, the terms “treat,” “treating,” and “treatment,” with respect to a disease, disorder or condition in a patient, refers to taking steps to obtain beneficial or desired results such as clinical results in the patient. Exemplary beneficial or desired clinical results include reduction or amelioration of the progression, severity, and/or duration of (a) the disease, disorder or condition, (b) one or more symptoms associated therewith, or (c) one or more symptoms resulting from the administration of one or more therapies (including, without limitation, administration of one or more prophylactic or therapeutic agents) directed to the disease, disorder or condition. It is understood that treatment of a disease, disorder or condition in a patient does not require that the patient manifests clinical symptoms. Rather, a patient can be identified as having an increased risk of developing clinical symptoms or progressing to an advanced stage of disease by retinal imaging and/or by one or more genetic markers and/or biomarkers, and the treatment can be used prophylactically for preventing or slowing clinical development of the disease, disorder or condition. For example, a patient who is diagnosed with intermediate AMD and identified as having an increased risk of progressing to geographic atrophy or neovascular AMD can be treated prophylactically for preventing or slowing the clinical progression.

As used herein, the terms “prevent,” “preventing,” and “prevention” refer to the prevention of the onset or recurrence of (a) a disease, disorder or condition, (b) one or more symptoms associated therewith, or (c) one or more symptoms resulting from the administration of one or more therapies (including, without limitation, administration of one or more prophylactic or therapeutic agents) directed to the disease, disorder or condition.

As used herein, the term “effective amount” refers to the amount of a substance (e.g., a protein of the present disclosure) sufficient to effect beneficial or desired results. An effective amount can be administered in one or more administrations, applications, or dosages and is not intended to be limited to a particular formulation, administration route, or frequency of administration.

As used herein, the term “ocular cell” refers to any cell in, or associated with the function of, the eye. The term may refer to any one or more of photoreceptor cells, including rod, cone and photosensitive ganglion cells, retinal pigment epithelium (RPE) cells, glial cells, Muller cells, bipolar cells, horizontal cells, amacrine cells. In one embodiment, the ocular cells are bipolar cells. In another embodiment, the ocular cells are horizontal cells. In another embodiment, the ocular cells are ganglion cells. In yet another embodiment, the ocular cells are RPE cells.

The terms “polypeptide,” “oligopeptide,” “peptide” and “protein” are used interchangeably herein to refer to chains of amino acids of any length. The chain may be linear or branched, it may comprise modified amino acids, and/or may be interrupted by non-amino acids. The terms also encompass an amino acid chain that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that the polypeptides can occur as single chains or associated chains.

As known in the art, “polynucleotide,” or “nucleic acid,” as used interchangeably herein, refer to chains of nucleotides of any length, and include DNA and RNA. The nucleotides can be deoxyribonucleotides, ribonucleotides, modified nucleotides or bases, and/or their analogs, or any substrate that can be incorporated into a chain by DNA or RNA polymerase. A polynucleotide may comprise modified nucleotides, such as methylated nucleotides and their analogs. If present, modification to the nucleotide structure may be imparted before or after assembly of the chain. The sequence of nucleotides may be interrupted by non-nucleotide components. A polynucleotide may be further modified after polymerization, such as by conjugation with a labeling component. Other types of modifications include, for example, “caps,” substitution of one or more of the naturally occurring nucleotides with an analog, internucleotide modifications such as, for example, those with uncharged linkages (e.g., methyl phosphonates, phosphotriesters, phosphoamidates, carbamates, etc.) and with charged linkages (e.g., phosphorothioates, phosphorodithioates, etc.), those containing pendant moieties, such as, for example, proteins (e.g., nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl, 2′-fluoro- or 2′-azido-ribose, carbocyclic sugar analogs, alpha- or beta-anomeric sugars, epimeric sugars such as arabinose, xyloses or lyxoses, pyranose sugars, furanose sugars, sedoheptuloses, acyclic analogs and abasic nucleoside analogs such as methyl riboside. One or more phosphodiester linkages may be replaced by alternative linking groups. These alternative linking groups include, but are not limited to, embodiments wherein phosphate is replaced by P(O)S (“thioate”), P(S)S (“dithioate”), (O)NR₂ (“amidate”), P(O)R, P(O)OR′, CO or CH₂ (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

Percent (%) sequence identity of a given amino acid sequence or nucleotide sequence (the “query sequence”), with respect to a reference sequence, is defined as the percentage of amino acid residues or nucleotides in the query sequence that are identical with the amino acid residues or nucleotides in the reference sequence after aligning the sequences (and introducing gaps, if necessary) to achieve the maximum percent sequence identity, not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared.

Unless otherwise defined herein, scientific and technical terms used in this application shall have the meanings that are commonly understood by those of ordinary skill in the art. Generally, nomenclature used in connection with, and techniques of, pharmacology, cell and tissue culture, molecular biology, cell and cancer biology, neurobiology, neurochemistry, virology, immunology, microbiology, genetics and protein and nucleic acid chemistry, described herein, are those well-known and commonly used in the art. In case of conflict, the present specification, including definitions, will control.

The practice of the present disclosure will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, second edition (Sambrook et al., 1989) Cold Spring Harbor Press; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R.I. Freshney, ed., 1987); Introduction to Cell and Tissue Culture (J.P. Mather and P.E. Roberts, 1998) Plenum Press; Cell and Tissue Culture: Laboratory Procedures (A. Doyle, J.B. Griffiths, and D. G. Newell, eds., 1993-1998) J. Wiley and Sons; Methods in Enzymology (Academic Press, Inc.); Gene Transfer Vectors for Mammalian Cells (J. M. Miller and M. P. Calos, eds., 1987); Current Protocols in Molecular Biology (F. M. Ausubel et al., eds., 1987); PCR: The Polymerase Chain Reaction, (Mullis et al., eds., 1994); Sambrook and Russell, Molecular Cloning: A Laboratory Manual, 3rd. ed., Cold Spring Harbor Laboratory Press, Cold Spring Harbor, N Y (2001); Ausubel et al., Current Protocols in Molecular Biology, John Wiley & Sons, N Y (2002); Harlow and Lane Using Antibodies: A Laboratory Manual, Cold Spring Harbor Laboratory Press, Cold Spring Harbor, N Y (1998); Coligan et al., Short Protocols in Protein Science, John Wiley & Sons, N Y (2003); Short Protocols in Molecular Biology (Wiley and Sons, 1999). The skilled worker would recognize many additional sources describing conventional techniques of molecular biology (including recombinant techniques), microbiology, cell biology, biochemistry and immunology.

Enzymatic reactions and purification techniques are performed according to manufacturer's specifications, as commonly accomplished in the art or as described herein. The nomenclatures used in connection with, and the laboratory procedures and techniques of, analytical chemistry, biochemistry, immunology, molecular biology, synthetic organic chemistry, and medicinal and pharmaceutical chemistry described herein are those well-known and commonly used in the art. Standard techniques are used for chemical syntheses, and chemical analyses.

Throughout this specification and embodiments, the word “comprise,” or variations such as “comprises” or “comprising,” will be understood to imply the inclusion of a stated integer or group of integers but not the exclusion of any other integer or group of integers. It is understood that wherever embodiments are described herein with the language “comprising,” otherwise analogous embodiments described in terms of “consisting of” and/or “consisting essentially of” are also provided.

The term “including” is used to mean “including but not limited to.” “Including” and “including but not limited to” are used interchangeably.

Any example(s) following the term “e.g.” or “for example” is not meant to be exhaustive or limiting.

Unless otherwise required by context, singular terms shall include pluralities and plural terms shall include the singular.

The articles “a” and “an” are used herein to refer to one or to more than one (i.e., to at least one) of the grammatical object of the article. By way of example, “an element” means one element or more than one element.

Reference to “about” a value or parameter herein includes (and describes) embodiments that are directed to that value or parameter per se. For example, description referring to “about X” includes description of “X.” Similarly, where description refers to a number “X,” description referring to “about X” is also contemplated. The term “about” or “approximately” can mean within an acceptable error range for the particular value as determined by one of ordinary skill in the art, which will depend in part on how the value is measured or determined, e.g., the limitations of the measurement system. For example, “about” can mean plus or minus 10%, per the practice in the art. Alternatively, “about” can mean a range of plus or minus 20%, plus or minus 10%, plus or minus 5%, or plus or minus 1% of a given value. When a value includes associated error measurements; the term “about” typically includes values encompassed within a range of +/−the stated error or +/−twice the stated error, or +/−three times the stated error. In case of doubt, encompassed within the term “about” are numbers that are insignificantly different from the stated number. Alternatively, particularly with respect to biological systems or processes, the term can mean within an order of magnitude, within 5-fold, or within 2-fold, of a value. Where particular values are described in the application and claims, unless otherwise stated, the term “about” meaning within an acceptable error range for the particular value can be assumed. Also, where ranges and/or subranges of values are provided, the ranges and/or subranges can include the endpoints of the ranges and/or subranges.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges subsumed therein. For example, a stated range of “1 to 10” should be considered to include any and all subranges between (and inclusive of) the minimum value of 1 and the maximum value of 10; that is, all subranges beginning with a minimum value of 1 or more, e.g., 1 to 6.1, and ending with a maximum value of 10 or less, e.g., 5.5 to 10.

Where aspects or embodiments of the disclosure are described in terms of a Markush group or other grouping of alternatives, the present disclosure encompasses not only the entire group listed as a whole, but each member of the group individually and all possible subgroups of the main group, but also the main group absent one or more of the group members. The present disclosure also envisages the explicit exclusion of one or more of any of the group members in the disclosure.

Each embodiment described herein may be used individually or in combination with any other embodiment described herein.

### Methods of Treatment

Disclosed herein are methods of treating, preventing, and/or ameliorating the progression of an ocular inflammatory disease, disorder, or condition, or retinal changes associated therewith, the method comprising administering CFH (e.g., a CFH protein or a vector encoding a CFH protein) to a subject in need thereof.

In certain embodiments, the subject has, or is at risk of developing a disease of the eye. A disease of the eye, includes, without limitation, AMD, retinitis pigmentosa, rod-cone dystrophy, Leber's congenital amaurosis, Usher's syndrome, Bardet-Biedl Syndrome, early-onset macular dystrophy, Best disease, retinoschisis, uveitis (e.g., anterior uveitis), sterile conjunctivitis, keratitis, Stargardt's disease (autosomal dominant or autosomal recessive), untreated retinal detachment, pattern dystrophy, cone-rod dystrophy, achromatopsia, ocular albinism, enhanced S cone syndrome, diabetic retinopathy, diabetic macular edema, retinopathy of prematurity, sickle cell retinopathy, Congenital Stationary Night Blindness, glaucoma, or retinal vein occlusion. In another embodiment, the subject has, or is at risk of developing glaucoma, Leber's hereditary optic neuropathy, lysosomal storage disorder, or peroxisomal disorder. In another embodiment, the subject has shown clinical signs of a disease of the eye.

An inflammatory ocular disease, disorder, or condition may exist simultaneously with a renal disease or complication in the same subject. In some embodiments, the subject has, or is at risk of developing a renal disease or complication. In some embodiments, the renal disease or complication is associated with AMD or atypical hemolytic uremic syndrome (aHUS). In some embodiments, the subject has, or is at risk of developing AMD or aHUS.

Clinical signs of a disease of the eye include, but are not limited to, decreased peripheral vision, decreased central (reading) vision, decreased night vision, loss of color perception, reduction in visual acuity, decreased photoreceptor function, and pigmentary changes. In one embodiment, the subject shows degeneration of the outer nuclear layer (ONL). In another embodiment, the subject has been diagnosed with a disease of the eye. In yet another embodiment, the subject has not yet shown clinical signs of a disease of the eye.

In certain embodiments, the inflammatory ocular disease, disorder, or condition is AMD. In certain embodiments, the AMD is dry AMD. In certain embodiments, the AMD is geographic atrophy (GA) secondary to dry AMD. In certain embodiments, the GA is multifocal. In certain embodiments, the GA that affects the foveal center point. In certain embodiments, the GA has a total size within 0.5 to 15.0 disk areas.

In some embodiments, the subject is a mammal. In some embodiments, the subject is a human. In some embodiments, the human is an adult. In some embodiments, the human is an elderly adult. In some embodiments, the human (e.g., the human who has or is at risk of age-related macular degeneration) is at least 40, 45, 50, 55, 60, 65, 70, 75, 80, 85, 90, or 95 years of age. In particular embodiments, the human is at least 50 years of age.

In some embodiments, the method disclosed herein is used in treatment of a patient having dry AMD. In certain embodiments, the patient has geographic atrophy. In certain embodiments, the area of geographic atrophy in an eye of the patient is from 2.5 to 17.5 mm² as determined by a method recognized by a clinician, for example, by screening images of fundus autofluorescence (FAF). In certain embodiments, the patient has multifocal geographic atrophy and at least one focal lesion has a size of 1.25 mm² or larger.

In certain embodiments, the subject has a best corrected visual acuity (BCVA) in the affected eye using Early Treatment Diabetic Retinopathy Study (ETDRS) chart VAS of 5-45 letters (equivalent to Snellen VA of approximately 20/800-20/125). In certain embodiments, the subject does not have any active ocular disease, disorder, or condition that could be a contraindication to intravitreal (IVT) injection.

The method disclosed herein may be used to prevent the occurrence of retinal damage or to rescue eyes having mild or advanced disease, i.e., to prevent progression of the disease to total blindness, prevent spread of damage to uninjured ocular cells, improve damage in injured ocular cells, or to provide enhanced vision. In one embodiment, the composition is administered before the disease becomes symptomatic or prior to photoreceptor loss. By symptomatic is meant onset of any of the various retinal changes described above or vision loss. In another embodiment, the composition is administered after disease becomes symptomatic. In yet another embodiment, the composition is administered after initiation of photoreceptor loss. In another embodiment, the composition is administered after outer nuclear layer (ONL) degeneration begins. In some embodiments, it is desirable that the composition is administered while bipolar cell circuitry to ganglion cells and optic nerve remains intact.

In another embodiment, the composition is administered after initiation of photoreceptor loss. In yet another embodiment, the composition is administered when less than 90% of the photoreceptors are functioning or remaining, as compared to a non-diseased eye. In another embodiment, the composition is administered when less than 80% of the photoreceptors are functioning or remaining. In another embodiment, the composition is administered when less than 70% of the photoreceptors are functioning or remaining. In another embodiment, the composition is administered when less than 60% of the photoreceptors are functioning or remaining. In another embodiment, the composition is administered when less than 50% of the photoreceptors are functioning or remaining. In another embodiment, the composition is administered when less than 40% of the photoreceptors are functioning or remaining. In another embodiment, the composition is administered when less than 30% of the photoreceptors are functioning or remaining. In another embodiment, the composition is administered when less than 20% of the photoreceptors are functioning or remaining. In another embodiment, the composition is administered when less than 10% of the photoreceptors are functioning or remaining. In one embodiment, the composition is administered only to one or more regions of the eye. In another embodiment, the composition is administered to the entire eye.

The present disclosure provides dosing regimens of CFH protein for treating the ocular inflammatory disease, disorder, or condition, or retinal changes associated therewith. The present disclosure also provides biomarkers for monitoring the response to a treatment of CFH (e.g., a CFH protein or a vector encoding a CFH protein) or for selecting a subject for such treatment. These aspects of the disclosure are provided in the “Dosage Regimen” section and the “Biomarkers” section below, respectively. A combination of these two aspects, directed to a method of treating an ocular inflammatory disease, disorder, or condition, or retinal changes associated therewith, following a dosing regimen disclosed herein based on one or more biomarker levels disclosed herein, is also contemplated.

### Dosing Regimen

The CFH or pharmaceutical composition disclosed herein is administered at an effective amount. The effective amount can be achieved by one or more doses of administration. In one aspect, the present disclosure provides a method of treating a subject having an inflammatory ocular disease, disorder, or condition, the method comprising administering to the subject an effective dose of CFH protein per eye, thereby treating the inflammatory ocular disease, disorder, or condition.

Doses of the CFH or pharmaceutical composition can be selected based on the route of administration. Routes of intraocular administration include but are not limited to intravitreal, subretinal, suprachoroidal, subconjunctival, retrobulbar, peribulbar, and intracameral administration. The specific doses disclosed herein are suitable for at least intravitreal administration. In some embodiments, the dose of a single administration in the eye is in the range of 10-1,000 μg/eye (e.g., 10-500 μg/eye, 10-250 μg/eye, 50-1,000 μg/eye, 50-500 μg/eye, 50-250 μg/eye, 100-1,000 μg/eye, 100-500 μg/eye, or 100-250 μg/eye). Informed by toxicological studies in cynomolgus monkeys and clinical and pre-clinical data from human patients, a new dosing regimen for the administration of CFH has been created to achieve less frequent dosing. In some embodiments, the dose for a single administration in the eye is about 50 μg/eye, about 100 μg/eye, about 250 μg/eye, or about 500 μg/eye. In some embodiments, the dose for a single administration in the eye is 50 μg/eye, 100 μg/eye, 250 μg/eye, or 500 μg/eye. It is understood that a higher dose, to the extent meeting safety criteria, is more desirable in clinical use given the expected advantage of less frequent dosing.

In certain embodiments, the CFH or pharmaceutical composition disclosed herein is administered at a fixed frequency. For example, doses may be administered in the eye (e.g., intravitreal administration or subretinal injection) weekly, biweekly, once every three weeks, monthly (or once every 30 days), once every two months (or once every 60 days), once every three months (or once every 90 days), once every four months, once every five months, once every six months, once every seven months, once every eight months, once every nine months, once every ten months, once every eleven months, or yearly, or even once every 2 to 20 years. In certain embodiments, the CFH or pharmaceutical composition disclosed herein is administered to the patient for at least two, at least three, at least four, at least five, or at least six doses.

In certain embodiments, a dose of 250 μg is administered once every month, once every 4 weeks, once every two months, or once every 8 weeks. In certain embodiments, a dose of 500 μg is administered once every month, once every 4 weeks, once every two months, or once every 8 weeks.

In certain embodiments, the CFH or pharmaceutical composition disclosed herein is to be administered at a variable frequency. Persons of ordinary skill in the art can estimate repetition rates for dosing based on measured residence times and concentrations of the active agent and/or biomarkers described herein in bodily fluids (e.g., ocular sample or plasma) or tissues.

For example, the protein level of one or more biomarkers disclosed herein relative to a predetermined threshold can inform the investigator/physician of the presence of the disease, disorder, or condition, whether the subject is a candidate for treatment with CFH, the severity of the disease, disorder, or condition, whether prior treatment with CFH is effective, and whether the dose, frequency, and/or mode of administration of a prior treatment with CFH is adequate or should be altered. Thus, the method disclosed herein includes obtaining or having obtained a measurement of the protein level of at least one biomarker for the purpose of diagnosis, treating, monitoring treatment, reducing progression, and/or selecting subjects for treatment with CFH. In certain embodiments, the subject is first administered complement factor H (CFH) in one or more initial doses, and afterwards, the protein level of at least one biomarker of a sample from the subject is obtained.

In certain embodiments, the concentration of CFH is also determined or obtained for assessing the patient's need for the next dose. In certain embodiments, CFH concentrations (e.g., total CFH concentration or concentration of the administered CFH) in an ocular sample are determined prior to or after treatment with CFH.

In certain embodiments, the method increases the level of CFH protein in an ocular sample (e.g., aqueous humor) of the subject. In certain embodiments, the CFH protein concentration in aqueous humor remains at the level of at least 100 ng/mL, at least 200 ng/mL, at least 300 ng/mL, at least 400 ng/mL, at least 500 ng/mL, at least 600 ng/mL, at least 700 ng/mL, at least 800 ng/mL, at least 900 ng/mL, or at least 1,000 ng/mL one month or 30 days after administration. In certain embodiments, the CFH protein concentration in aqueous humor remains at the level of at least 100 ng/mL, at least 200 ng/mL, at least 300 ng/mL, at least 400 ng/mL, at least 500 ng/mL, at least 600 ng/mL, at least 700 ng/mL, at least 800 ng/mL, at least 900 ng/mL, or at least 1,000 ng/mL two months or 60 days after administration.

Additional methods of assessing the progression or severity of the inflammatory ocular disease, disorder, or condition, for the purpose of assessing the patient's need for the next dose, are described in the “therapeutic efficacy” subsection infra.

### Biomarkers

Disclosed herein are methods of treating, preventing, ameliorating, diagnosing, and/or monitoring the progression of an ocular inflammatory disease, disorder, or condition or retinal changes associated therewith, based on the levels of one or more biomarkers.

In one aspect, the present disclosure provides a method for treating a subject having an inflammatory ocular disease, disorder, or condition, the method comprising: (a) obtaining or having obtained a measurement of the protein level of at least one biomarker disclosed herein in a sample (e.g., an ocular sample) of the subject; (b) determining whether the measurement is greater than or lower than a predetermined threshold; and (c) administering to the subject CFH if the protein level of the biomarker is (i) greater than or equal to the threshold if the biomarker is positively correlated with activation of the complement pathway or with inflammation or (ii) is lower than or equal to the threshold if the biomarker is negatively correlated with activation of the complement pathway or with inflammation, thereby treating the inflammatory ocular disease, disorder, or condition.

In one aspect, the present disclosure provides a method for identifying a subject having an inflammatory ocular disease, disorder, or condition suitable for treatment with CFH, the method comprising: (a) obtaining or having obtained a measurement of the protein level of at least one biomarker disclosed herein in a sample (e.g., an ocular sample) of the subject; and (b) determining whether the measurement is greater than or lower than a predetermined threshold, wherein the subject is suitable for treatment with CFH if the protein level of the biomarker is (i) greater than or equal to the threshold if the biomarker is positively correlated with activation of the complement pathway or with inflammation or (ii) is lower than or equal to the threshold if the biomarker is negatively correlated with activation of the complement pathway or with inflammation.

In another aspect, the present disclosure provides a method for treating a subject having an inflammatory ocular disease, disorder, or condition, the method comprising: (a) selecting a subject whose protein level of at least one biomarker disclosed herein in a sample (e.g., an ocular sample) of the subject is (i) greater than or equal to a predetermined threshold if the biomarker is positively correlated with activation of the complement pathway or with inflammation or (ii) is lower than or equal to a predetermined threshold if the biomarker is negatively correlated with activation of the complement pathway or with inflammation; and (b) administering to the subject CFH, thereby treating the inflammatory ocular disease, disorder, or condition.

In another aspect, the present disclosure provides a method for selecting a subject for treatment of an inflammatory ocular disease, disorder, or condition, the method comprising: (a) obtaining or having obtained a measurement of the protein level of at least one biomarker disclosed herein in a sample (e.g., an ocular sample) of the subject; (b) determining whether the protein level of the biomarker is greater than or lower than a predetermined threshold; and (c) selecting the subject for treatment of the inflammatory ocular disease, disorder, or condition if the protein level of the biomarker is (i) greater than or equal to the threshold if the biomarker is positively correlated with activation of the complement pathway or with inflammation or (ii) is lower than or equal to the threshold if the biomarker is negatively correlated with activation of the complement pathway or with inflammation, wherein the treatment comprises administering to the subject CFH.

The protein level of a biomarker selected from the group consisting of (i) complement component C3, complement factor B (CFB), complement component C5, and a cleavage fragment thereof, (ii) proteins associated with inflammation; and (iii) proteins associated with choroidal neovascularization may be used to monitor the response to a treatment disclosed herein and/or to determine when to provide the next dose of treatment. In certain embodiments, the protein levels of one or more biomarkers (e.g., one or more aqueous humor complement protein and/or complement split products) are used to evaluate the efficacy of administration of CFH (e.g., a CFH protein or a vector encoding a CFH protein) of the present disclosure to a subject diagnosed with an ocular disease or disorder. In certain embodiments, the protein levels of one or more biomarkers (e.g., one or more aqueous humor complement protein and/or complement split products) are evaluated before and after treatment from aqueous humor samples obtained from the subjects who are administered the CFH or protein of the present disclosure. In certain embodiments, the protein levels of one or more biomarkers (e.g., one or more aqueous humor complement protein and/or complement split products) are evaluated before and after treatment from plasma samples obtained from the subjects who are administered the CFH (e.g., a CFH protein or a vector encoding a CFH protein) of the present disclosure.

In certain embodiments, the subject selected for the treatment based on the biomarker profile has been diagnosed of an inflammatory ocular disease, disorder, or condition by one or more clinical standards. In certain embodiments, the subject selected for the treatment based on the biomarker profile does not meet all the clinical standards of an inflammatory ocular disease, disorder, or condition, but is otherwise identified to be at high risk of developing such inflammatory ocular disease, disorder, or condition (e.g., according to genetic test results).

The alternative pathway of the complement system must be tightly controlled by a group of alternative pathway regulators to prevent host cell and tissue destruction. One endogenous mechanism often employed to regulate excessive alternative pathway activity is to prevent convertase formation by degrading the core component C3b to a proteolytic by-product that is incapable of forming the convertase. The activity of the alternative pathway is indicated, for example, by the formation and protease activity of C3-convertase, serine protease factor D (CFD), C5-convertase, which cleave many proteins in the complement system. For example, C3 is a substrate of C3-convertase and the cleavage products include C3a and C3b. CFB is a substrate of CFD and the cleavage products include Ba and Bb. C5 is a substrate of C5-convertase and the cleavage products include C5a and C5b. It is contemplated that the activity of the alternative pathway can be used as a criterion to select a subject who is more likely to respond to a CFH therapy.

In some embodiments, the biomarker is a cleavage fragment of C3, CFB, or C5. In some embodiments, the biomarker is selected from the group consisting of C3a, Ba, Bb, C5a, C5b, and sC5b-9. The protein levels of these biomarkers are positively correlated with activation of the complement pathway. Therefore, in these embodiments, the subject is treated with CFH or selected for the treatment if the protein level of the biomarker is greater than or equal to the threshold. Median Ba and C3a concentration levels of patients diagnosed with dry AMD are ˜2-fold greater than that of healthy patients (see Schick et al., “Local complement activation in aqueous humor in patients with age-related macular degeneration,” Eye, 1-4 (2017) and Altay et al., “Early local activation of complement in aqueous humor in patients with age-related macular degeneration,” Eye (Lond), 33(12):1859-1864 (2019).

In certain embodiments, the biomarker is C3a and the predetermined threshold is 1 ng/mL, 1.1 ng/mL, 1.2 ng/mL, 1.3 ng/mL, 1.4 ng/mL, 1.5 ng/mL, 1.6 ng/mL, 1.7 ng/mL, 1.8 ng/mL, 1.9 ng/mL, 2 ng/mL, 2.1 ng/mL, 2.2 ng/mL, 2.3 ng/mL, 2.4 ng/mL, 2.5 ng/mL, 2.6 ng/mL, 2.7 ng/mL, 2.8 ng/mL, 2.9 ng/mL, 3 ng/mL, 3.1 ng/mL, 3.2 ng/mL, 3.3 ng/mL, 3.4 ng/mL, 3.5 ng/mL, 3.6 ng/mL, 3.7 ng/mL, 3.8 ng/mL, 3.9 ng/mL, 4 ng/mL, 4.1 ng/mL, 4.2 ng/mL, 4.3 ng/mL, 4.4 ng/mL, 4.5 ng/mL, 4.6 ng/mL, 4.7 ng/mL, 4.8 ng/mL, 4.9 ng/mL, 5 ng/mL, 6 ng/mL, 7 ng/mL, 8 ng/mL, 9 ng/mL, or 10 ng/mL. In certain embodiments, subjects with elevated C3a concentration levels showed reduction in C3a concentration levels after administration of CFH or the protein of present disclosure. In certain embodiments, C3a concentration levels are reduced at least 10%, at least 20%, at least 30%, at least 40%, at least 50%, at least 60%, at least 70%, at least 80%, at least 90% in patients with elevated C3a levels after administration of CFH or the protein of present disclosure. In certain embodiments, C3a concentration levels do not reduce below 3000 μg/mL as measured by a MicroVue C3a Plus Enzyme Immunoassay Kit after administration of CFH or the protein of present disclosure.

In certain embodiments, the biomarker is Ba and the predetermined threshold is 6 ng/mL, 7 ng/mL, 8 ng/mL, 9 ng/mL, 10 ng/mL, 11 ng/mL, 12 ng/mL, 13 ng/mL, 14 ng/mL, 15 ng/mL, 16 ng/mL, 17 ng/mL, 18 ng/mL, 19 ng/mL, 20 ng/mL, 21 ng/mL, 22 ng/mL, 23 ng/mL, 24 ng/mL, 25 ng/mL, 30 ng/mL, 40 ng/mL, or 50 ng/mL. In certain embodiments, subjects with elevated Ba concentration levels showed reduction in Ba concentration levels after administration of CFH or the protein of present disclosure. In certain embodiments, Ba concentration levels are reduced at least 10%, at least 20%, at least 30%, at least 40%, at least 50%, at least 60%, at least 70%, at least 80%, at least 90% in patients with elevated Ba levels after administration of CFH or the protein of present disclosure. In certain embodiments, Ba concentration levels do not reduce below 10 ng/mL, below 9 ng/mL, below 8 ng/mL, below 7 ng/mL, or below 6 ng/mL as measured by a MicroVue Ba fragment EIA Kit after administration of CFH or the protein of present disclosure.

In certain embodiments, the biomarker is C5a and the predetermined threshold is C5a and the predetermined threshold is 100 pg/mL, 150 pg/mL, 200 pg/mL, 250 pg/mL, 300 pg/mL, 400 pg/mL, 500 pg/mL, 600 pg/mL, 700 pg/mL, 800 pg/mL, 900 pg/mL, or 1 ng/mL.

In some embodiments, the biomarker is uncleaved CFB, or C5. The protein levels of these biomarkers are negatively correlated with activation of the complement pathway. Therefore, in these embodiments, the subject is treated with CFH or selected for the treatment if the protein level of the biomarker is lower than or equal to the threshold. In certain embodiments, the biomarker is CFB and the predetermined threshold is 10 ng/mL, 20 ng/mL, 30 ng/mL, 40 ng/mL, 50 ng/mL, 60 ng/mL, 70 ng/mL, 80 ng/mL, 90 ng/mL, 100 ng/mL, 200 ng/mL, 300 ng/mL, 400 ng/mL, 500 ng/mL, 600 ng/mL, 700 ng/mL, 800 ng/mL, 900 ng/mL, or 1 μg/mL. In certain embodiments, the biomarker is C5 and the predetermined threshold is 10 ng/mL, 20 ng/mL, 30 ng/mL, 40 ng/mL, 50 ng/mL, 60 ng/mL, 70 ng/mL, 80 ng/mL, 90 ng/mL, 100 ng/mL, 110 ng/mL, 120 ng/mL, 130 ng/mL, 140 ng/mL, 150 ng/mL, 160 ng/mL, 170 ng/mL, 180 ng/mL, 190 ng/mL, 200 ng/mL, or 250 ng/mL.

In some embodiments, the biomarker is CFH. The protein level of this biomarker is negatively correlated with activation of the complement pathway. Therefore, in these embodiments, the subject is treated with CFH or selected for the treatment if the protein level of the biomarker is lower than or equal to the threshold. In certain embodiments, the biomarker is CFH and the predetermined threshold is 10 ng/mL, 15 ng/mL, 20 ng/mL, 25 ng/mL, 30 ng/mL, 35 ng/mL, 40 ng/mL, 45 ng/mL, 50 ng/mL, 55 ng/mL, or 60 ng/mL. In certain embodiments, a subject with a reduced CFH concentration in an ocular sample (e.g., aqueous humor) shows an increase in CFH concentration after administration of CFH. In certain embodiments, the CFH concentration in the ocular sample (e.g., aqueous humor) is increased at least 2-fold, at least 3-fold, at least 4-fold, at least 5-fold, at least 6-fold, at least 7-fold, at least 8-fold, at least 9-fold, at least 10-fold, at least 11-fold, at least 12-fold, at least 13-fold, at least 14-fold, at least 15-fold, at least 20-fold, at least 25-fold in the patient with reduced CFH level after administration of CFH. In certain embodiments, the CFH concentration in aqueous humor remains at the level of at least 50 ng/mL, at least 100 ng/mL, at least 200 ng/mL, at least 300 ng/mL, at least 400 ng/mL, at least 500 ng/mL, at least 600 ng/mL, at least 700 ng/mL, at least 800 ng/mL, at least 900 ng/mL, or at least 1,000 ng/mL one month or 30 days after administration. In certain embodiments, the CFH concentration in aqueous humor remains at the level of at least 50 ng/mL, at least 100 ng/mL, at least 200 ng/mL, at least 300 ng/mL, at least 400 ng/mL, at least 500 ng/mL, at least 600 ng/mL, at least 700 ng/mL, at least 800 ng/mL, at least 900 ng/mL, or at least 1,000 ng/mL two months or 60 days after administration. It is understood that reduced levels of CFH protein in ocular samples are often observed in patients having an ocular disease, disorder, or condition associated with choroidal neovascularization (CNV) who have been treated with VEGF (e.g., VEGF-A) antagonists. Exemplary ocular diseases, disorders, and conditions associated with CNV include neovascular AMD (i.e., wet AMD) and diabetic retinopathy (e.g., diabetic macular edema). Such patients are susceptible to developing macular atrophy and are suitable for treatment with a CFH therapy. Accordingly, in certain embodiments, CFH is used as a biomarker to select a patient having an ocular disease, disorder, or condition associated with CNV and who has been treated with an VEGF (e.g., VEGF-A) antagonist. In certain embodiments, the patient has received a VEGF antagonist treatment by ocular administration. In certain embodiments, the patient received a VEGF antagonist treatment within one month, two months, three months, four months, five months, six months, or a year before the administration of the CFH therapy. In certain embodiments, the patient continues to receive a VEGF antagonist during the treatment with the CFH therapy. In certain embodiments, the patient received a VEGF antagonist treatment 10 minutes, 15 minutes, 20 minutes, 25 minutes, 30 minutes, 35 minutes, 40 minutes, 50 minutes, 60 minutes, 120 minutes, 140 minutes, 240 minutes before the administration of the CFH therapy.

Patients having or at risk of developing an ocular disease, disorder, or condition associated with CNV, such as neovascular AMD (i.e., wet AMD) and diabetic retinopathy (e.g., diabetic macular edema), can also be selected based on the protein level of one or more biomarkers associated with CNV. Exemplary biomarkers positively associated with CNV include VEGF-A. In certain embodiments, the biomarker is VEGF-A and the predetermined threshold of VEGF-A is 100 pg/mL, 110 pg/mL, 120 pg/mL, 130 pg/mL, 140 pg/mL, 150 pg/mL, 160 pg/mL, 170 pg/mL, 180 pg/mL, 190 pg/mL, 200 pg/mL, 250 pg/mL, 300 pg/mL, 350 pg/mL, 400 pg/mL, 450 pg/mL, or 500 pg/mL. It is contemplated that CFH is suitable for treating such patients without causing choroidal neovascularization as many other treatments for geographic atrophy or macular atrophy do. In certain embodiments, the patient has not received a VEGF antagonist treatment. In other embodiments, the patient has received a VEGF antagonist treatment but still exhibit an elevated level of VEGF-A relative to the threshold level.

It is contemplated that patients having or at risk of developing an ocular disease, disorder, or condition associated with CNV, including but not limited to patient selected based on the protein levels of CFH and/or VEGF-A, can be treated by a combination therapy of CFH and a VEGF antagonist without causing choroidal neovascularization. Accordingly, in certain embodiments, the method disclosed herein comprises (a) obtaining or having obtained a measurement of the protein level of CFH and/or a protein associated with choroidal neovascularization (e.g., VEGF-A); (b) determining whether the protein level of the biomarker is greater than or lower than a predetermined threshold as disclosed herein; and (c) administering to the subject an effective amount of CFH and an effective amount of a VEGF antagonist if the protein level of the biomarker is (i) greater than or equal to the threshold if the biomarker is positively correlated with activation of the complement pathway or with inflammation or (ii) lower than or equal to the threshold if the biomarker is negatively correlated with activation of the complement pathway or with inflammation, thereby treating the inflammatory ocular disease, disorder, or condition. Combination therapies of CFH and a VEGF antagonist are disclosed in more detail in the Combination Therapy subsection below. In certain embodiments, the patient has an ocular disease, disorder, or condition associated with CNV, such as neovascular AMD (i.e., wet AMD) and diabetic retinopathy (e.g., diabetic macular edema). In certain embodiments, the patient is selected for the therapy if the protein level of a protein associated with choroidal neovascularization meets the selection standard (e.g., the protein level of VEGF-A is greater than or equal to a predetermined threshold disclosed herein). In certain embodiments, the patient is selected for the therapy if the protein level of CFH is lower than or equal to a predetermined threshold disclosed herein. It is understood that a combination of CFH and a VEGF antagonist can be administered to a patient prophylactically before its ocular CFH protein level reaches or falls below the threshold. Accordingly, in certain embodiments, the patient is selected for the therapy if the protein level of CFH is greater than the threshold. In certain embodiments, obtaining the protein level of CFH is dispensable.

It is understood that activation of the complement system may result in release of host cell components, which may in turn enhance inflammation. Therefore, it is contemplated that the activity of inflammation can be used as a criterion to select a subject who is more likely to respond to a CFH therapy. Exemplary biomarkers positively associated with ocular inflammation include interleukin (IL)-1β, IL-6, IL-8, IL-10, IL-18, TNF-α, C-C motif chemokine ligand 2 (CCL2, a.k.a. monocyte chemotactic protein 1 or MCP-1), C-X-C motif chemokine 5 (CXCL5), C-C motif chemokine 24 (CCL24, a.k.a. Eotaxin-2), and uncleaved C3. Accordingly, in certain embodiments, the protein associated with ocular inflammation is selected from the group consisting of IL-10, IL-6, IL-8, IL-10, IL-18, TNF-α, CCL2, CXCL5, Eotaxin-2, and uncleaved C3, and the subject is treated with CFH or selected for the treatment if the protein level of the biomarker is greater than or equal to the threshold.

In certain embodiments, the biomarker is IL-1 and the predetermined threshold of IL-10 is 0.2 pg/mL, 0.3 pg/mL, 0.4 pg/mL, 0.5 pg/mL, 0.6 pg/mL, 0.7 pg/mL, 0.8 pg/mL, 0.9 pg/mL, or 1 pg/mL. In certain embodiments, the biomarker is IL-6 and the predetermined threshold of IL-6 is 4 pg/mL, 5 pg/mL, 6 pg/mL, 7 pg/mL, 8 pg/mL, 9 pg/mL, 10 pg/mL, 15 pg/mL, or 20 pg/mL. In certain embodiments, the biomarker is IL-8 and the predetermined threshold of IL-8 is 9 pg/mL, 10 pg/mL, 11 pg/mL, 12 pg/mL, 13 pg/mL, 14 pg/mL, 15 pg/mL, 20 pg/mL, 25 pg/mL, 30 pg/mL, 40 pg/mL, 50 pg/mL, 60 pg/mL, 70 pg/mL, 80 pg/mL, 90 pg/mL, or 100 pg/mL. In certain embodiments, the biomarker is IL-10 and the predetermined threshold of IL-10 is 1.2 pg/mL, 1.3 pg/mL, 1.4 pg/mL, 1.5 pg/mL, 1.6 pg/mL, 1.7 pg/mL, 1.8 pg/mL, 1.9 pg/mL, 2 pg/mL, 2.5 pg/mL, 3 pg/mL, 3.5 pg/mL, 4 pg/mL, or 5 pg/mL. In certain embodiments, the biomarker is IL-18 and the predetermined threshold of IL-18 is 5 pg/mL, 10 pg/mL, 15 pg/mL, 20 pg/mL, 25 pg/mL, 30 pg/mL, 35 pg/mL, 40 pg/mL, 45 pg/mL, 50 pg/mL, 60 pg/mL, 70 pg/mL, or 80 pg/mL. In certain embodiments, the biomarker is TNF-α and the predetermined threshold of TNF-α is 0.3 pg/mL, 0.35 pg/mL, 0.4 pg/mL, 0.45 pg/mL, or 0.5 pg/mL. In certain embodiments, the biomarker is CCL2 and the predetermined threshold of CCL2 is 2 ng/mL, 2.5 ng/mL, 3 ng/mL, 3.5 ng/mL, 4 ng/mL, 4.5 ng/mL, 5 ng/mL, 5.5 ng/mL, 6 ng/mL, 6.5 ng/mL, 7 ng/mL, 7.5 ng/mL, 8 ng/mL, 8.5 ng/mL, 9 ng/mL, 9.5 ng/mL, or 10 ng/mL. In certain embodiments, the biomarker is CXCL5 and the predetermined threshold of CXCL5 is 1 pg/mL, 2 pg/mL, 3 pg/mL, 4 pg/mL, 5 pg/mL, 6 pg/mL, 7 pg/mL, 8 pg/mL, 9 pg/mL, 10 pg/mL, 15 pg/mL, 20 pg/mL, 25 pg/mL, 30 pg/mL, 40 pg/mL, or 50 pg/mL. In certain embodiments, the biomarker is Eotaxin-2 and the predetermined threshold of Eotaxin-2 is 10 pg/mL, 20 pg/mL, 30 pg/mL, 40 pg/mL, 50 pg/mL, 60 pg/mL, 70 pg/mL, 80 pg/mL, 90 pg/mL, 100 pg/mL, 110 pg/mL, 120 pg/mL, 130 pg/mL, 140 pg/mL, 150 pg/mL, 160 pg/mL, 170 pg/mL, 180 pg/mL, 190 pg/mL, or 200 pg/mL. In certain embodiments, the biomarker is uncleaved C3 and the predetermined threshold of uncleaved C3 is 100 ng/mL, 200 ng/mL, 300 ng/mL, 400 ng/mL, 500 ng/mL, 600 ng/mL, 700 ng/mL, 800 ng/mL, 900 ng/mL, 1 μg/mL, 1.1 μg/mL, 1.2 μg/mL, 1.3 μg/mL, 1.4 μg/mL, or 1.5 μg/mL.

A plurality of biomarkers disclosed herein can be used for the patient selection. In certain embodiments, the method comprises obtaining or determining the protein levels of 2, 3, 4, 5, 6, 7, 8, 9, 10, 11, 12, 13, 14, 15 or more biomarkers disclosed herein.

In certain embodiments, the method comprises obtaining or determining the protein levels of 2, 3, 4, 5, 6, 7, 8, 9, 10, 11, 12, 13, 14, 15 or more complement components or fragments thereof. In certain embodiments, the method described herein comprises obtaining or determining the protein levels of a panel of biomarkers comprising a complement component and a cleavage fragment thereof. In certain embodiments, the method described herein comprises obtaining or determining the protein levels of a panel of biomarkers comprising two or more (e.g., three or more) biomarkers selected from C5, CFB, C3a, and Ba. In certain embodiments, the method described herein comprises obtaining or determining the protein levels of a panel of biomarkers comprising C3a and Ba. In certain embodiments, the method described herein comprises obtaining or determining the protein levels of a panel of biomarkers comprising C5, CFB, C3a, and Ba.

In certain embodiments, the method comprises obtaining or determining the protein levels of 2, 3, 4, 5, 6, 7, 8, 9, 10, 11, 12, 13, 14, 15 or more proteins associated with ocular inflammation. In certain embodiments, the method comprises obtaining or determining the protein levels of a panel of biomarkers comprising two or more (e.g., three or more, four or more, five or more, six or more, seven or more, eight or more, or nine or more) biomarkers selected from TNF, IL-6, IL-8, IL-10, IL-18, IL-10, CCL2, CXCL5, Eotaxin-2, and C3.

In certain embodiments, the method comprises obtaining or determining the protein levels of one or more (e.g., 2, 3, 4, 5, 6, 7, 8, 9, 10, 11, 12, 13, 14, 15 or more) complement components or fragments thereof, one or more (e.g., 2, 3, 4, 5, 6, 7, 8, 9, 10, 11, 12, 13, 14, 15 or more) proteins associated with ocular inflammation, and/or one or more (e.g., 2, 3, 4, 5, 6, 7, 8, 9, 10, 11, 12, 13, 14, 15 or more) proteins associated with ocular inflammation proteins associated with ocular neovascularization. In certain embodiments, the method comprises obtaining or determining the protein levels of one or more (e.g., 2, 3, 4, 5, 6, 7, 8, 9, 10, 11, 12, 13, 14, 15 or more) complement components or fragments thereof and one or more (e.g., 2, 3, 4, 5, 6, 7, 8, 9, 10, 11, 12, 13, 14, 15 or more) proteins associated with ocular inflammation, and optionally VEGF-A. In certain embodiments, the method comprises obtaining or determining the protein levels of a panel of biomarkers comprising two or more (e.g., three or more, four or more, five or more, six or more, seven or more, eight or more, nine or more, ten or more, 11 or more, 12 or more, 13 or more, or 14 or more) biomarkers selected from C3, C5, B, C3a, Ba, TNF, IL-6, IL-8, IL-1β, IL-18, IL-10, CCL2, CXCL5, Eotaxin-2, and VEGF-A. In certain embodiments, the method comprises obtaining or determining the protein levels of a panel of biomarkers comprising C3, C5, B, C3a, Ba, TNF, IL-6, IL-8, IL-1β, IL-18, IL-10, CCL2, CXCL5, Eotaxin-2, and VEGF-A. Where multiple biomarkers are assessed, patient selection can be made based on a score that takes into account the protein levels of the multiple biomarkers and the relative impact of each biomarker on the risk of disease development and progression.

The biomarker panels described herein can include biomarkers in addition to any of those described herein, including biomarkers that when having a level above or below a predetermined threshold is an indication that the subject has an ocular inflammatory disease, would benefit from CFH treatment, an inflammatory ocular disease has progressed, and/or a higher dose of CFH is needed for treating, reducing progression, or preventing progression of an ocular inflammatory disease.

The sample of the subject can be obtained from various tissues. In certain embodiments, the sample of the subject is an ocular sample. In certain embodiments, the ocular sample comprises aqueous humor. In certain embodiments, the sample comprises vitreous humor. In other embodiments, the sample of the subject is a serum sample. In certain embodiments, both an ocular sample and serum sample are obtained.

Aqueous humor can be collected via a technique called anterior chamber paracentesis (ACP). This involves the use of a 27- or 30-gauge needle attached to a 1 ml syringe that is inserted at the paralimbal clear cornea in a plane above and parallel to the iris with the bevel of the needle facing forward until the whole bevel has penetrated the cornea. Under direct vision, the retina physician then pulls the plunger of the syringe to aspirate a small quantity of aqueous humor. In certain embodiments, 10-300 μL of aqueous humor is obtained from subject. In certain embodiments, no more than 100 μL of aqueous humor is acquired from subjects. In certain embodiments, approximately 10 μL, 20 μL, 30 μL, 40 μL, 50 μL, 60 μL, 70 μL, 80 μL, 90 μL, or 100 μL of aqueous humor is acquired from a human subject.

### Detection and Quantification Assays

Any assay known in the art that has sufficient sensitivity to detect and quantify the biomarkers described herein in either an ocular sample (e.g., aqueous humor or vitreous humor) or serum sample can be used, including, but not limited to, mass spectrometry (MS), multiplex bead immunoassay (e.g., Luminex™ assay, Quanterix™ SIMOA bead technology), enzyme-linked immunoabsorbent assay (ELISA), electrochemiluminescence immunoassay (ECLIA), chemiluminescent microparticle immunoassay (CMIA), radioimmunoassay (RIA), immunodiffusion, immunoelectrophoresis, immunohistochemistry (IHC), flow cytometry, immunoprecipitation assays, complement fixation assays, binding with indexed antibodies in solutions and suspensions, protein arrays using antibodies, or any combinations thereof. In certain embodiments, the assay used for determination of the protein level of a biomarker described herein has a detection limit lower than or equal to 10 μg/mL, lower than or equal to 1 μg/mL, lower than or equal to 0.1 μg/mL, lower than or equal to 10 ng/mL, lower than or equal to 1 ng/mL, lower than or equal to 0.1 ng/mL, lower than or equal to 10 pg/mL, or lower than or equal to 1 pg/mL.

In certain embodiments, the protein level of a biomarker is obtained using a assay that requires a binding agent (e.g., a detection reagent, or a capture reagent in a sandwich-type immunoassay) that specifically binds the biomarker. In certain embodiments, the binding agent is an antibody. In certain embodiments, where the biomarker of interest is a cleavage fragment (e.g., C3a, Ba, Bb, C5a, and sC5b-9), the binding agent (e.g., antibody) specifically binds the cleavage fragment and not the precursor protein, or binds the cleavage fragment at a higher affinity than that to the corresponding precursor protein (e.g., the dissociation constant of the complex with the precursor protein is greater than that with the fragment by at least 5 fold, 10 fold, 20 fold, 50 fold, or 100 fold). For example, for detection of C3a, an antibody that has a higher affinity to C3a than to full-length C3 can be used.

In certain embodiments, a sandwich-type immunoassay method such as ELISA, ECLIA, CMIA, or RIA is used to quantify the protein level of a biomarker. For example, the method may comprise adding a biological sample to a first antibody combined with a bead, a film, a slide, or a microtiter plate made with a solid substrate, e.g., glass, plastic such as polystyrene, polysaccharide, nylon, or nitrocellulose and then qualitatively or quantitatively detecting a protein (e.g., a protein that directly or indirectly binds the analyte) labeled with a directly or indirectly detectable moiety. Such moieties include, without limitation, radioactive substances such as ³H or ¹²⁵, fluorescent substances, chemiluminescent substances, electrochemiluminescent substances, hapten, biotin, digoxigenin, enzymes such as horseradish peroxidase (HRP), alkaline phosphatase (ALP), and malate dehydrogenase capable of colorimetry or luminescence through an reaction with a substrate. Exemplary ELISA methods are described in Gaastra, W., Methods in Molecular Biology, Vol. 1, Walker, J. M. ed., Humana Press, N J, 1984. Exemplary ECLIA methods are described in Prieto et al., (2010) Clin. Chem. Lab. Med. 48:835-38 and have been developed for commercial use by companies such as Meso Scale Discovery (MSD).

In certain embodiments, a dipstick assay is used to quantify the protein level of a biomarker. The dipstick assay is a technology widely used in the field of point of care test (POCT). It is a chromatic method for detecting a biomarker, showing color when a biomarker of interest in a sample binds an antibody in the substrate (e.g., nitrocellulose) and combines with a detection antibody due to capillarity when the sample contacts the substrate, e.g., when one end of a dipstick is dipped into the sample.

In certain embodiments, concentration levels of C3a in human aqueous humor samples are quantified using a MicroVue C3a Plus Enzyme Immunoassay Kit (Quidel, cat #A031). This is a 96 well, direct capture immunoassay where a microassay plate is coated with a murine monoclonal antibody specific for human C3a. When standards, controls, and samples are added to this plate, the antibody on the plate binds to C3a. After the incubation period, a wash cycle removes any unbound material. Next a horseradish peroxidase (HRP)-conjugated anti-C3a is added to each assay well where it binds to the immobilized C3a captured in the first step. When a 3,3′,5,5′ tetramethylbenzidine (TMB), a ready-to-use, chromogenic substrate solution, is added to the assay wells, the bound HRP reacts with the substrate forming a blue color. After the incubation period, the reaction is stopped and the color intensity is measured spectrophotometrically at A450. The color intensity of the reaction mixture is proportional to the concentration of C3a present in the standards, controls, and samples. Results are calculated from the generated standard curve using 5-parameter analysis.

In certain embodiments, concentration levels of Ba in human aqueous humor samples are quantified using a MicroVue Ba fragment EIA Kit (Quidel, cat #A033). Based on quantitative ELISA format, a microtiter plate is coated with a mouse mAb that specifically binds to human Ba. When standards, controls, and samples are added to the wells, the Ba fragments present in them bind to the immobilized Ab. A horse radish peroxidase (HRP) conjugated polyclonal anti-Ba Ab is used as the detection reagent. This is detected by TMB substrate which will generate color which is directly proportional to the amount of Ba in the samples.

In certain embodiments, CFH, Factor B, and C3 concentration levels in human aqueous humor samples are quantified using a Luminex-Human Complement Magnetic Bead Panel 2 Kit (Millipore cat #HCMP2MAG-19K). This is a double capture multiplex bead-based Immunoassay which uses spectrally encoded magnetic beads conjugated to specific antibodies, e.g., for CFH, anti-factor H capture antibodies are used as the solid support. FH in samples and QC will bind to these antibodies conjugated to color-coded beads. The bound FH is detected with biotinylated anti-FH antibody producing a Magnetic bead antibody-antigen(Ag)-antibody double capture complex. When a reporter molecule, streptavidin-conjugated to Phycoerythrin (Streptavidin-PE) is added to the mixture, a fluorescence is generated which can be read on a BioPlex Suspension Array instrument. By monitoring the Spectral properties of the magnetic beads and the PE Fluorescence intensity (FI), the concentration of Factor H can be determined.

In certain embodiments, CFH concentration levels in human plasma are quantified using a MicroVue Factor H EIA (Quidel, cat #A039). This is a quantitative Elisa method where the microtiter plate is coated with a mouse monoclonal antibody that binds specifically to human Factor H. When standards, controls, or specimens are added to the plate, FH will bind to the immobilized anti-Factor H monoclonal antibody. Next, a horseradish peroxidase (HRP)-conjugated murine anti-Factor H antibody is added to each well as the detection reagent which will bind to the FH captured on the plate. When a chromogenic enzyme substrate (TMB) is added, the bound HRP-conjugate reacts with the substrate, forming a color which is measured spectrophotometrically at A450. The color intensity of the reaction mixture is proportional to the concentration of Factor H present in the samples.

In certain embodiments, immunoelectrophoresis such as Ouchterlony plate, western blot, crossed IE, rocket IE, fused rocket IE, or affinity IE, wherein a biomarker may easily detect through antigen-antibody binding, is used to quantify the protein level of a biomarker described herein. Exemplary immunoassays are described in Enzyme Immunoassay, E. T. Maggio, ed., CRC Press, Boca Raton, Florida, 1980.

In certain embodiments, microparticles or nanoparticles are used in immunoassays to quantify the protein level of a biomarker. Microparticles are often in suspension and may be synthesized in large batches, which reduces labor and allows for higher standardization between assays. Suspension assays also allow for easier mixing, leading to faster reaction times on microspheres. For multiplexed analyses, microspheres can be added or removed to individually choose each protein for the assay. Fluorescence microbeads can be analyzed by various methods, including flow cytometry, laser scanning cytometer or confocal laser scanning microspectrometer. For example, Luminex™ polystyrene beads can be applied to immunoassays. Because each bead serves as a separate replicate and many beads can be fabricated simultaneously in batch, bead-based immunoassays may be more reliable, more reproducible, and cheaper than other immunoassays. Another advantage of microparticles is the ability to trap and concentrate analyte molecules for improved sensitivity. This improved sensitivity is particularly important in microfluidic and point of care devices where detector sensitivity may be compromised because of size limitations.

In certain embodiments, an array including a microarray or a chip method is used to detect the protein level of a biomarkers described herein. Array techniques are known in the art and can be found in, for example, Schena et al., 1996, Proc Natl Acad Sci USA. 93 (20):10614-9; Schena et al., 1995, Science 270(5235):467-70; and U.S. Pat. Nos. 5,599,695, 5,556,752, and 5,631,734. A detection reagent may be attached to the surface of a substrate such as glass or nitrocellulose. The detection reagent that may be attached to an array may include an antibody, an antibody fragment, an aptamer, an avidity multimer, or peptidomimetics capable of being specifically binding with one protein.

In certain embodiments, the protein level of a biomarker is assessed using mass spectrometry (MS) (see, e.g., Kim, et al. 2010 J Proteome Res. 9: 689-99; Anderson, L et al. 2006. Mol Cell Proteomics 5: 573-88). Mass spectrometry can detect a wide range of proteins in a single measurement without labeling and can be performed from a biological sample, such as an ocular sample or serum. In an embodiment, for example, multiple reaction monitoring (MRM) by utilizing a triple quadrupole LC-MS/MS, QTRAP, etc. is used. The MRM is a method for quantitative measuring of a substance, such as a small amount of biomarker present in biological samples, in multiple ways, in which precursor ions or parent ions among ion fragments are created from an ionization source selectively and sent to a collision cell by using a first mass filter Q1. After that, the precursor ions in the collision cell collide with internal collision gas and splits into product ions or daughter ions, which are sent to a second mass filter Q2. Only characteristic ions are sent to a detecting unit. This is an analytic method with high selectivity and high sensitivity that may detect information on required ingredients only (see Gillette et al., 2013, Nature Methods 10:28-34).

When mass spectrometry techniques are used to detect and quantify one or more biomarkers in a test sample, the test sample must first be prepared for mass spectrometry analysis. Sample preparation can take place in several ways, but the one that is most commonly used involves contacting the sample with one or more adsorbents attached to a solid phase. The adsorbents may be anionic or cationic groups, hydrophobic groups, metal chelating groups with or without a metal ligand, antibodies, whether polyclonal or monoclonal, or suitable antigens to bind their related antibodies. The solid phase can be a flat surface made of metal, glass, or plastic. The solid phase can also be microparticulate in nature, be they microbeads, amorphous particulates, or insoluble polymers to increase the surface area. In addition, microparticulate materials can be magnetic for handling. In certain embodiments, the biomarker of interest is absorbed in the solid phase and the remaining molecules are washed away. By mass analysis, the biomarker of interest is eluted from the solid phase with a solvent that reduces the affinity of the biomarker for the adsorbent. The biomarkers are then introduced into the mass spectrometer for analysis. Preferably, the peripheral spectra are identified and omitted in the spectrum evaluation. Additionally, immunoassays such as those described above can also be used. Upon termination of an immunoassay, the analyte can be eluted from the immune surface and introduced into the spectrophotometer for analysis.

Once the test sample has been prepared, it is introduced into the mass analyzer. Laser ionization desorption (for example, MALDI or SELDI) is a common technique for samples that are presented in solid form. In this technique, the sample is co-crystallized on a target plate with an effective matrix to absorb and transfer the laser energy to the sample. The ions are separated, counted, and calibrated against the ions of known mass and charge. The mass data collected by any sample is an ionic count with a specific mass/charge ratio (m/z). It is anticipated that different sample preparation methods and different ionization techniques will result in different spectra.

In certain embodiments, the detection of the biomarker can be performed through detection of a peptide derived from a biomarker protein. For example,

Certain MS methods are suitable for detecting shorter peptides or polypeptides. To analyze a protein of interest having a large size, the sample can be digested with one or more proteases to generate fragments of the protein, and signature fragments can be detected by MS. Where the event to be detected is cleavage of a protein (e.g., C3, CFB, or C5), this technology is particularly suitable for detecting the depletion of a signature fragment of the uncleaved protein. Accordingly, in specific embodiments, the protein level of C3, CFB, or C5 is measured by MS.

It is understood that the volume of an ocular sample is typically small, for example, no more than 100 μL. As a result, an assay suitable for measurement of the levels of one or more proteins in an ocular sample is generally highly sensitive and/or compatible with small sample volumes. Given the potential benefit of assessing more than one biomarker, in certain embodiments, the assay is a multiplex assay. In certain embodiments, the protein level of the biomarker is measured by a multiplex bead immunoassay (e.g., Luminex™ assay, Quanterix™ SIMOA bead technology). In certain embodiments, the protein level of the biomarker is measured by electrochemiluminescence immunoassay (ECLIA).

### Genetic Patient Selection Criteria

In lieu of or in addition to the biomarker levels disclosed above, one or more of the genetic criteria below can be used for patient selection.

In some embodiments, the method disclosed herein is used in treatment of a patient having one or more CFH mutations. The presence or absence of any of the CFH mutations disclosed herein can be determined by genetic testing. In some embodiments, the patient has one or more CFH mutations that causes macular degeneration (AMD) or that increases the likelihood that a patient develops AMD. In some embodiments, the patient has one or more mutations that causes atypical hemolytic uremic syndrome (aHUS) or that increases the likelihood that a patient develops aHUS. In some embodiments, the one or more mutations are in the patient's CFH gene. In some embodiments, the subject has a loss-of-function mutation in the subject's CFH gene.

In particular embodiments, the patient expresses a mutant CFH protein, wherein the mutant CFH protein has reduced CFH activity as compared to a wildtype CFH protein (e.g., a CFH protein having the amino acid sequence of SEQ ID NO: 1, 2 or 3). In some embodiments, the CFH activity is the ability to bind to C3b. In some embodiments, the CFH activity is the ability to act as a cofactor with CFI and facilitate C3b cleavage. In some embodiments, the CFH activity is the ability to bind to a cell surface (e.g., an erythrocyte and/or endothelial cell). In some embodiments, the CFH activity is the ability to bind to heparin. In some embodiments, the CFH activity is the ability to inhibit C5b9 levels as a result of complement activation, e.g., as measured in a Wieslab AP assay (see, e.g., Example 1). In some embodiments, the CFH activity is the ability to inhibit hemolysis. In some embodiments, if the mutant CFH protein were tested in a functional assay, the mutant CFH protein would display reduced CFH activity as compared to a wildtype CFH protein (e.g., a CFH protein having the amino acid sequence of SEQ ID NO: 1, 2, or 3). Examples of CFH mutants associated with reduced CFH activity include R2T, R53C, R53H, S58A, D130N, R175Q, R175P, I221V, R303W, R303Q, Y402H, P503A, R567G, G650V, G1194D, or R1210C CFH mutants. As used herein, the amino acid positions in a CFH protein is numbered according a full-length CFH protein including the signal peptide, for example, the amino acid sequence set forth NCBI Reference Sequence NP_000177.2. In some embodiments, if a cell expresses less of a variant CFH polypeptide than the same cell would express a wildtype control CFH polypeptide, then the variant CFH is determined to be functionally impaired. In some embodiments, the patient is homozygous for any of the mutations disclosed herein. In some embodiments, the patient is heterozygous for any of the mutations disclosed herein.

In some embodiments, the patient is homozygous for the Y402H mutation of the CFH gene. In some embodiments, the patient homozygous for the Y402H mutation of the CFH gene is negative for (a) the ARMS2/HTRA1 risk allele of ARMS2: c298-858T>C in the rs3750846 SNP; (b) homozygous CFH 62V, homozygous C3 102G, and homozygous CFB 32R; and/or (c) rare missense CFH mutations selected from the group consisting of R2T, L3V, R53C, R53H, S58A, D90G, D130N, R175Q, R175P, I221V, R303W, R303Q, Q400K, P503A, R567G, G650V, S890I, T956M, G1194D, and R1210C. In some embodiments, the patient homozygous for the Y402H mutation of the CFH gene is negative for any of the mutations in (a), (b), and (c) above.

In some embodiments, the patient is homozygous for CFH 62V, C3 102G, and CFB 32R.

In some embodiments, the patient is positive for at least one of the rare missense CFH mutations selected from the group consisting of R2T, L3V, R53C, R53H, S58A, D90G, D130N, R175Q, R175P, I221V, R303W, R303Q, Q400K, P503A, R567G, G650V, S890I, T956M, G1194D, and R1210C.

In some embodiments, the patient has one or more mutations in the patient's CFH gene. In some embodiments, the treatment and/or prophylactic method is for use in treating a patient in whom it has been determined has one or more of any of the CFH mutations disclosed herein. In some embodiments, the patient has a mutation in one or more of CCP domains 1-20, or any combination thereof. In some embodiments, the patient has a mutation in one or more of CCP domains 1-2 or 18-20. In some embodiments, the patient has a mutation in CCP1. In some embodiments, the patient has a mutation in CCP2. In some embodiments, the patient has a mutation in CCP3. In some embodiments, the patient has a mutation in CCP4. In some embodiments, the patient has a mutation in CCP5. In some embodiments, the patient has a mutation in CCP6. In some embodiments, the patient has a mutation in CCP7. In some embodiments, the patient has a mutation in CCP8. In some embodiments, the patient has a mutation in CCP9. In some embodiments, the patient has a mutation in CCP10. In some embodiments, the patient has a mutation in CCP11. In some embodiments, the patient has a mutation in CCP12. In some embodiments, the patient has a mutation in CCP13. In some embodiments, the patient has a mutation in CCP14. In some embodiments, the patient has a mutation in CCP15. In some embodiments, the patient has a mutation in CCP16. In some embodiments, the patient has a mutation in CCP17. In some embodiments, the patient has a mutation in CCP18. In some embodiments, the patient has a mutation in CCP19. In some embodiments, the patient has a mutation in CCP20. In some embodiments, the patient has one or more mutations in the disulphide bond sites in the CFH protein. In some embodiments, the mutation is one or more of the mutations selected from the group consisting of: H402Y, G69E, D194N, W314C, A806T, Q950H, p.Ile184fsX, p.Lys204fsX, c.1697-17_-8del, A161S, A173G, R175Q, V62I, V1007L, S890I, S193L, I216T, A301Nfs*25, W379R, Q400K, Q950H, T956M, R1210C, N1050Y, E936D, Q408X, R1078S, c.350+6T→G, R567G, R53C, R53H, R2T, A892V, R567G, I221V, S159N, P562H, F960S, R303W, R303Q, K666N, G1194D, P258L, G650V, D130N, S58A, R166W, R232Q, R127H, K1202N, G397Stop, Stop450R, R830W, I622L, T732M, S884Y, L24V, Y235H, K527N, R582H, C973Y, V1089M, E123G, T291S, R567K, E625Stop, N802S, N1056K, R1203W, Q1076E, P26S, T46A, T91S, C129Y, R166Q, E167Q, R175P, C192F, W198*, V206M, G218*, M239T, Y277*, C325Y, R341H, R364L, P384R, C431S, D454A, A473V, P503A, N516K, I551T, H699R, F717L, W978R, P981S, A1010V, W1037*, P1051L, I1059T, Q1143E, R1206H, T1227I, L24V, H169R, R257H, K410E, V609I, D619N, A892V, G1002R, G278S, T30*, I32Stop, R78G, Q81P, V111E, W134R, P139S, M162V, E189Stop, K224Del, K224Del, A307A, H332Y, S411T, C448Y, L479Stop, R518T, T519A, C536R, C564P, C569Stop, L578Stop, P621T, C623S, C630W, E635D, K670T, Q672Q, C673Y, C673S, S714Stop, S722*, C733Y, V737V, E762Stop, N774Stop, R780I, G786*, M823T, V835L, E847V, E850K, C853R, C853T, C864S, C870R, H878H, I881L, E889Stop, H893R, Y899Stop, Y899D, C915S, C915Stop, W920R, Q925Stop, C926F, Y951H, C959Y, P968*, I970V, T987A, N997T, G1011*, T1017I, Y1021F, C1043R, T1046T, V1054I, V1060A, V1060L, C1077W, T1097W, T1097T, D1119G, D1119N, P1130L, V1134G, E1135R, E1137L, E1139Stop, Y1142D, Y1142C, C1152S, W1157R, P1161T, C1163T, P1166L, V1168E, V1168Stop, I1169L, E1172Stop, Y1177C, R1182S, W1183L, W1183R, W1183L, W1183Stop, W1183C, T1184R, T1184A, K1186H, K1188Del, L1189R, L1189F, S1191L, S1191W, E1195Stop, V1197A, E1198A, E1198Stop, F1199S, V1200L, G1204E, L1207R, S1211P, R1215Q, R1215G, T1216Del, C1218R, Y1225*, P1226S, L3V, H821Y, E954del, G255E, T1038R, V383A, V641A, P213A, I221V, E229K, R2T, R1072G, G967E, N819S, V579F, G19K, A18S, K834E, T504M, R662I, P668L, G133R, I184T, L697F, H1165Y, G1110A, pIle808_Gln809del, I760L, T447R, I808M, I868M, L765F, N767S, R567G, K768N, S209L, Q628K, D214Y, N401D, I216K, Q464R, I777V, E229D, M823I, R232Ter, S266L, P260S, E23G, C80Y, R78T, R582H, N638D, N638S, P258L, L3F, R257H, G240R, G69R, D855N, M11I, K472N, Q840H, E850K, Y899H, T645M, M805V, K919T, E201G, V407A, I907L, T914K, H332R, V144M, S652G, D195N, C146S, P661R, E677Q, V482I, T34R, A421T, R281G, C509Y, K666N, P440S, C442G, N607D, A425V, G667E, P440L, I49V, R387G, E625K, E625Ter, T135S, P43S, K283E, I124V, T36V, I563T, G350E, D619G, T321I, T286A, P384L, T739N, M515L, V158A, G727R, T724K, F717L, M162V, C178R, G700R, A161T, F176L, R295S, F298Y, G297S, P300L, R1040K, V552L, T310I, T531A, G928D, Ter386RextTer 69d€, Q1143K, Y534C, P981L, K308N, D538E, R1215Ter, E105V, T1017I, N1050I, P935S, Y951H, T1097M, D947H, E961D, G962S, G964E, I970V, R1072T, P1114L, S1122T, F960C, R1074C, R1182T, R1074L, S884Y, S890T, V837I, V941F, V158I, D748V, I216T, H371N, L750F, P418T, M432V, D693N, A746E, V111E, c.2237-2A>G, P982S, V579A, E591D, V579I, V65I, P418S, Y1067C, D772N, V72L, E189K, A1027P, D798N, N61D, P384S, N521S, P1068S, E395K, N774S, H577R, E833K, K6E, H337R, R444C, L741F, Y42F, D288E, S705F, R1040G, D214H, N757D, I861M, G848E, P923S, E201K, E902A, R303Q, G366E, D538H, K82R, E721K, Y1008H, R1074P, A806S, Q807R, C389Y, H764Y, K867N, P392T, L394M, E456K, F459L, Y398C, E570K, D214N, I574V, I574T, G631C, T880I, V865F, V576A, N776S, P633S, N22D, P634A, N822I, R885S, R232L, E635D, R778K, L827V, C267R, Y779C, R582C, L77S, R257C, Y327H, N75K, L74F, S836T, Y243H, c.1519+5_1519+8delGT . . . , K507Q, A892S, I15T, P924L, A14V, N842K, G894R, G894E, Y271C, C9W, T504R, V683M, L385Phed€, S898R, Q408H, G409S, T34K, E648G, I412V, E338D, P799S, G480E, D798E, D195Y, R341C, D485H, D485G, K598Q, Y420H, P599T, N434H, R441T, C431G, V149A, V349I, T679A, P43T, G45D, R662G, T519I, L121P, P364L, P621A, H373Y, D538MfsTer14, H371P, T544A, T131A, R166G, V177I, V177A, R729S, F717V, N718S, S991G, L98I, Y1016Ter, T1217del, M1001T, K1004E, A1010T, G1011D, T1017A, T1031A, L1125F, R1203G, L1214M, W1096DfsTer20, H939N F960L, D966H, M1064I, E1071K, N1095K, T1106A, G1107E, C1109W, P1111S, V1197I, Y1075F, S1079N, P1080S, E1082G, or Sto1232. In particular embodiments, the mutation is one or more of the mutations selected from the group consisting of: R2T, L3V, R53C, R53H, S58A, G69E, D90G, R175Q, S193L, I216T, I221V, R303W, H402Y, Q408X, P503A, G650V, R1078S, and R1210C. As used herein, any of the CFH mutant amino acid positions described herein correspond to the amino acid CFH sequence of full-length human CFH including the signal peptide (UniProt Accession No. P08603-1).

In some embodiments, the patient has any one of the following CFH mutations: R2T, L3V, R53C, R53H, S58A, D90G, D130N, R175Q, R175P, I221V, R303W, R303Q, Q400K, Y402H, P503A, R567G, G650V, S890I, T956M, G1194D, or R1210C. In some embodiments, the patient has any one or more of the following CFH mutations: R2T, R53C, R53H, S58A, D130N, R175Q, R175P, I221V, R303W, R303Q, P503A, R567G, G650V, G1194D, or R1210C. In some embodiments, the patient has an R2T mutation. In some embodiments, the patient has an L3V mutation. In some embodiments, the patient has an R53C mutation. In some embodiments, the patient has an R53H mutation. In some embodiments, the patient has an S58A mutation. In some embodiments, the patient has a D90G mutation. In some embodiments, the patient has a D130N mutation. In some embodiments, the patient has an R175Q mutation. In some embodiments, the patient has an R175P mutation. In some embodiments, the patient has an I221V mutation. In some embodiments, the patient has an R303W mutation. In some embodiments, the patient has an R303Q mutation. In some embodiments, the patient has a Q400K mutation. In some embodiments, the patient has a Y402H mutation. In some embodiments, the patient has a P503A mutation. In some embodiments, the patient has an R567G mutation. In some embodiments, the patient has a G650V mutation. In some embodiments, the patient has an S890I mutation. In some embodiments, the patient has a T956M mutation. In some embodiments, the patient has a G1194D mutation. In some embodiments, the patient has an R1210C mutation.

In certain embodiments, the CFH mutation is a change from the Tyr at amino acid position 402 of human CFH protein to a Histidine and is present in at least one of the two alleles of the CFH gene in the genome of the patient. In certain embodiments, the CFH mutation is a mutation of the Tyr at amino acid position 402 of human CFH protein and is present in both alleles of the CFH gene. In certain embodiments, the genetic variant comprises SNP rs1061170 in at least one allele of the human genome. In certain embodiments, the genetic variant comprises rs1061170 in both alleles of the human genome.

In some embodiments, the patient expresses less CFH, or less functional CFH, than expressed in a cell or tissue (e.g., eye) than a control patient or population thereof. In some embodiments, the control patient does not have any of the CFH mutations disclosed herein. In some embodiments, the control patient does not have a genetic mutation that impairs CFH function. In some embodiments, the control patient is of the same or similar age and/or of the same sex as the patient selected. In some embodiments, the control patient is a healthy subject, e.g., the subject does not have a disease, disorder, or condition of the eye. In some embodiments, the control patient does not have a disease or disorder of the eye associated with activation of the complement cascade. In some embodiments, the control patient does not have macular degeneration. In some embodiments, the eye or a specific cell type of the eye (e.g., cells in the foveal region) in the selected patient expresses at least 95%, 90%, 80%, 70%, 60%, 50%, 40%, 30%, 20%, 10%, 5%, or 1% less CFH or functional CFH as compared to the levels in the control patient. In some embodiments, the eye or a specific cell type of the eye (e.g., cells in the foveal region) in the selected patient express CFH protein having any one or more of the CFH mutations disclosed herein. In some embodiments, the eye or a specific cell type of the eye (e.g., cells in the foveal region) in the control patient do not express a CFH protein having any of the CFH mutations disclosed herein.

### Complement Factor H Protein

The present disclosure provides a complement factor H (CFH) protein, including a biologically active fragment and/or variant of a full-length, wild-type CFH protein, for use in any of the methods disclosed herein. In certain embodiments, the CFH protein is a recombinant protein.

Mature human wild-type CFH is a 1213 amino acid soluble protein which comprises 20 complement-control protein modules (CCPs 1-20), which are each approximately 60 amino acid residues in length. Alignment of the 20 CCPs demonstrates four invariant cysteine residues arranged in two conserved disulfide bonds, and a near-invariant tryptophan residue. Short three to eight amino acid residue “linkers” are found between the last residue of one CCP and the first residue of the next CCP. Each of the CCPs fold into a distinct three-dimensional β-sheet rich structure (Schmidt C. Q. Clin Exp Immunol. 2008; 151(1):14-24). CCP module 1 corresponds to amino acids 1-64 of SEQ ID NO: 1 or a variant thereof; CCP module 2 corresponds to amino acids 65-125 of SEQ ID NO: 1 or a variant thereof; CCP module 3 corresponds to amino acids 126-189 of SEQ ID NO: 1 or a variant thereof; CCP module 4 corresponds to amino acids 190-246 of SEQ ID NO: 1 or a variant thereof; CCP module 5 corresponds to amino acids 247-304 of SEQ ID NO: 1 or a variant thereof; CCP module 6 corresponds to amino acids 306-368 of SEQ ID NO: 1 or a variant thereof; CCP module 7 corresponds to amino acids 369-426 of SEQ ID NO: 1 or a variant thereof; CCP module 8 corresponds to amino acids 428-489 of SEQ ID NO: 1 or a variant thereof; CCP module 9 corresponds to amino acids 497-548 of SEQ ID NO: 1 or a variant thereof; CCP module 10 corresponds to amino acids 549-607 of SEQ ID NO: 1 or a variant thereof; CCP module 11 corresponds to amino acids 610-668 of SEQ ID NO: 1 or a variant thereof; CCP module 12 corresponds to amino acids 671-728 of SEQ ID NO: 1 or a variant thereof; CCP module 13 corresponds to amino acids 733-787 of SEQ ID NO: 1 or a variant thereof; CCP module 14 corresponds to amino acids 791-848 of SEQ ID NO: 1 or a variant thereof; CCP module 15 corresponds to amino acids 850-910 of SEQ ID NO: 1 or a variant thereof; CCP module 16 corresponds to amino acids 911-968 of SEQ ID NO: 1 or a variant thereof; CCP module 17 corresponds to amino acids 969-1,027 of SEQ ID NO: 1 or a variant thereof; CCP module 18 corresponds to amino acids 1,028-1,086 of SEQ ID NO: 1 or a variant thereof; CCP module 19 corresponds to amino acids 1,089-1,147 of SEQ ID NO: 1 or a variant thereof; and CCP module 20 corresponds to amino acids 1,152-1,212 of SEQ ID NO: 1 or a variant thereof.

In some embodiments, the CFH protein comprises at least four CCP modules or a biologically active fragment and/or variant thereof. In some embodiments, the CFH protein comprises at least five CCP modules or a biologically active fragment and/or variant thereof. In some embodiments, the CFH protein comprises at least six CCP modules or a biologically active fragment and/or variant thereof. In some embodiments, the CFH protein comprises at least seven CCP modules or a biologically active fragment and/or variant thereof. In some embodiments, the CFH protein comprises at least eight CCP modules or a biologically active fragment and/or variant thereof. In some embodiments, the CFH protein comprises at least nine CCP modules or a biologically active fragment and/or variant thereof. In some embodiments, the CFH protein comprises at least ten CCP modules or a biologically active fragment and/or variant thereof. In some embodiments, the CFH protein comprises at least eleven CCP modules or a biologically active fragment and/or variant thereof. In some embodiments, the CFH protein comprises at least twelve CCP modules or a biologically active fragment and/or variant thereof. In some embodiments, the CFH protein comprises at least thirteen CCP modules or a biologically active fragment and/or variant thereof. In some embodiments, the CFH protein comprises at least fourteen CCP modules or a biologically active fragment and/or variant thereof. In some embodiments, the CFH protein comprises at least fifteen CCP modules or a biologically active fragment and/or variant thereof. In some embodiments, the CFH protein comprises at least sixteen CCP modules or a biologically active fragment and/or variant thereof. In some embodiments, the CFH protein comprises at least seventeen CCP modules or a biologically active fragment and/or variant thereof. In some embodiments, the CFH protein comprises at least eighteen CCP modules or a biologically active fragment and/or variant thereof. In some embodiments, the CFH protein comprises at least nineteen CCP modules or a biologically active fragment and/or variant thereof. In some embodiments, the CFH protein comprises twenty CCP modules or a biologically active fragment and/or variant thereof.

In some embodiments, the CFH protein comprises any one of or any combination of CCP modules 1, 2, 3, 4, 5, 6, 7, 8, 9, 10, 11, 12, 13, 14, 15, 16, 17, 18, 19, and/or 20 or a biologically active fragment and/or variant thereof. In some embodiments, the CFH protein comprises at least four of CCP modules 1, 2, 3, 4, 5, 6, 7, 8, 9, 10, 11, 12, 13, 14, 15, 16, 17, 18, 19, and/or 20 or a biologically active fragment and/or variant thereof. In some embodiments, the CFH protein comprises CCP modules 1-4, 1-5, 1-6, 1-7, 1-8, 1-9, 1-10, 1-11, 1-12, 1-13, 1-14, 1-15, 1-16, 1-17, 1-18, 1-19, or 1-20 or a biologically active fragment and/or variant thereof. In some embodiments, the CFH protein comprises CCP modules 2-20, 3-20, 4-20, 5-20, 6-20, 7-20, 8-20, 9-20, 10-20, 11-20, 12-20, 13-20, 14-20, 15-20, 16-20, 17-20, 18-20, or 19-20 or a biologically active fragment and/or variant thereof. In some embodiments, the CFH protein comprises CCP modules 1-4 and 19-20, or a biologically active fragment and/or variant thereof. In some embodiments, the CFH protein comprises CCP modules 1-4 and 18-20, or a biologically active fragment and/or variant thereof. In some embodiments, the CFH protein comprises CCP modules 1-5 and 18-20, or a biologically active fragment and/or variant thereof.

A consensus sequence of a full-length CFH protein is shown in SEQ ID NO: 1. Exemplary full-length CFH proteins covered by this consensus sequence are shown in Table 1.

TABLE 1
Amino Acid Substitutions in the CFH Sequence.

| | Amino Acid Position | | |
|---|---|---|---|
| SEQ ID NO | 62 | 402 | 936 |
| 1 | X₁ | X₂ | X₃ |
| 2 | V | Y | E |
| 3 | V | H | E |
| 4 | V | Y | D |
| 5 | V | H | D |
| 6 | I | Y | E |
| 7 | I | H | E |
| 8 | I | Y | D |
| 9 | I | H | D |

In some embodiments, the CFH protein comprises an amino acid sequence that is at least 70%, 75%, 80%, 85%, 90%, 91%, 92%, 93%, 94%, 95%, 96%, 97%, 98%, 99% or 100% identical to the amino acid sequence of SEQ ID NO: 1, or a biologically active fragment thereof. In some embodiments, the CFH protein comprises an amino acid sequence that is at least 90% identical to the amino acid sequence of SEQ ID NO: 1, or a biologically active fragment thereof. In some embodiments, the CFH protein comprises an amino acid sequence that is at least 95% identical to the amino acid sequence of SEQ ID NO: 1, or a biologically active fragment thereof. In some embodiments, the CFH protein comprises the amino acid sequence of SEQ ID NO: 1 or a biologically active fragment thereof.

In some embodiments, the CFH protein comprises an amino acid sequence that is at least 70%, 75%, 80%, 85%, 90%, 91%, 92%, 93%, 94%, 95%, 96%, 97%, 98%, 99% or 100% identical to the amino acid sequence of SEQ ID NO: 2 (mature CFH amino acid sequence (62V, 402Y, 936E)), or a biologically active fragment thereof. In some embodiments, the CFH protein comprises an amino acid sequence that is at least 90% identical to the amino acid sequence of SEQ ID NO: 2, or a biologically active fragment thereof. In some embodiments, the CFH protein comprises an amino acid sequence that is at least 95% identical to the amino acid sequence of SEQ ID NO: 2, or a biologically active fragment thereof. In some embodiments, the CFH protein comprises the amino acid sequence of SEQ ID NO: 2 or a biologically active fragment thereof.

In some embodiments, the CFH protein comprises an amino acid sequence that is at least 70%, 75%, 80%, 85%, 90%, 91%, 92%, 93%, 94%, 95%, 96%, 97%, 98%, 99% or 100% identical to the amino acid sequence of SEQ ID NO: 3 (mature CFH amino acid sequence (62V, 402H, 936E)), or a biologically active fragment thereof. In some embodiments, the CFH protein comprises an amino acid sequence that is at least 90% identical to the amino acid sequence of SEQ ID NO: 3, or a biologically active fragment thereof. In some embodiments, the CFH protein comprises an amino acid sequence that is at least 95% identical to the amino acid sequence of SEQ ID NO: 3, or a biologically active fragment thereof. In some embodiments, the CFH protein comprises the amino acid sequence of SEQ ID NO: 3 or a biologically active fragment thereof.

In some embodiments, the CFH protein comprises an amino acid sequence that is at least 70%, 75%, 80%, 85%, 90%, 91%, 92%, 93%, 94%, 95%, 96%, 97%, 98%, 99% or 100% identical to the amino acid sequence of SEQ ID NO: 4 (mature CFH amino acid sequence (62V, 402Y, 936D)), or a biologically active fragment thereof. In some embodiments, the CFH protein comprises an amino acid sequence that is at least 90% identical to the amino acid sequence of SEQ ID NO: 4, or a biologically active fragment thereof. In some embodiments, the CFH protein comprises an amino acid sequence that is at least 95% identical to the amino acid sequence of SEQ ID NO: 4, or a biologically active fragment thereof. In some embodiments, the CFH protein comprises the amino acid sequence of SEQ ID NO: 4 or a biologically active fragment thereof.

In some embodiments, the CFH protein comprises an amino acid sequence that is at least 70%, 75%, 80%, 85%, 90%, 91%, 92%, 93%, 94%, 95%, 96%, 97%, 98%, 99% or 100% identical to the amino acid sequence of SEQ ID NO: 5 (mature CFH amino acid sequence (62V, 402H, 936D)), or a biologically active fragment thereof. In some embodiments, the CFH protein comprises an amino acid sequence that is at least 90% identical to the amino acid sequence of SEQ ID NO: 5, or a biologically active fragment thereof. In some embodiments, the CFH protein comprises an amino acid sequence that is at least 95% identical to the amino acid sequence of SEQ ID NO: 5, or a biologically active fragment thereof. In some embodiments, the CFH protein comprises the amino acid sequence of SEQ ID NO: 5 or a biologically active fragment thereof.

In some embodiments, the CFH protein comprises an amino acid sequence that is at least 70%, 75%, 80%, 85%, 90%, 91%, 92%, 93%, 94%, 95%, 96%, 97%, 98%, 99% or 100% identical to the amino acid sequence of SEQ ID NO: 6 (mature CFH amino acid sequence (62I, 402Y, 936E)), or a biologically active fragment thereof. In some embodiments, the CFH protein comprises an amino acid sequence that is at least 90% identical to the amino acid sequence of SEQ ID NO: 6, or a biologically active fragment thereof. In some embodiments, the CFH protein comprises an amino acid sequence that is at least 95% identical to the amino acid sequence of SEQ ID NO: 6, or a biologically active fragment thereof. In some embodiments, the CFH protein comprises the amino acid sequence of SEQ ID NO: 6 or a biologically active fragment thereof.

In some embodiments, the CFH protein comprises an amino acid sequence that is at least 70%, 75%, 80%, 85%, 90%, 91%, 92%, 93%, 94%, 95%, 96%, 97%, 98%, 99% or 100% identical to the amino acid sequence of SEQ ID NO: 7 (mature CFH amino acid sequence (62I, 402H, 936E)), or a biologically active fragment thereof. In some embodiments, the CFH protein comprises an amino acid sequence that is at least 90% identical to the amino acid sequence of SEQ ID NO: 7, or a biologically active fragment thereof. In some embodiments, the CFH protein comprises an amino acid sequence that is at least 95% identical to the amino acid sequence of SEQ ID NO: 7, or a biologically active fragment thereof. In some embodiments, the CFH protein comprises the amino acid sequence of SEQ ID NO: 7 or a biologically active fragment thereof.

In some embodiments, the CFH protein comprises an amino acid sequence that is at least 70%, 75%, 80%, 85%, 90%, 91%, 92%, 93%, 94%, 95%, 96%, 97%, 98%, 99% or 100% identical to the amino acid sequence of SEQ ID NO: 8 (mature CFH amino acid sequence (62I, 402Y, 936D)), or a biologically active fragment thereof. In some embodiments, the CFH protein comprises an amino acid sequence that is at least 90% identical to the amino acid sequence of SEQ ID NO: 8, or a biologically active fragment thereof. In some embodiments, the CFH protein comprises an amino acid sequence that is at least 95% identical to the amino acid sequence of SEQ ID NO: 8, or a biologically active fragment thereof. In some embodiments, the CFH protein comprises the amino acid sequence of SEQ ID NO: 8 or a biologically active fragment thereof.

In some embodiments, the CFH protein comprises an amino acid sequence that is at least 70%, 75%, 80%, 85%, 90%, 91%, 92%, 93%, 94%, 95%, 96%, 97%, 98%, 99% or 100% identical to the amino acid sequence of SEQ ID NO: 9 (mature CFH amino acid sequence (62I, 402H, 936D)), or a biologically active fragment thereof. In some embodiments, the CFH protein comprises an amino acid sequence that is at least 90% identical to the amino acid sequence of SEQ ID NO: 9, or a biologically active fragment thereof. In some embodiments, the CFH protein comprises an amino acid sequence that is at least 95% identical to the amino acid sequence of SEQ ID NO: 9, or a biologically active fragment thereof. In some embodiments, the CFH protein comprises the amino acid sequence of SEQ ID NO: 9 or a biologically active fragment thereof.

In certain embodiments, the rCFH protein is characterized by an electrophoretogram comprising peak isoelectric point (pI) values from above 5.9 to below 6.8, wherein the pI values are measured by a capillary isoelectric focusing (cIEF) assay. In certain embodiments, the peak pI values are: about 6.02±0.03, about 6.06±0.03, about 6.11±0.02, about 6.16±0.02, about 6.22±0.02, about 6.28±0.02, about 6.33±0.01, about 6.40±0.01, about 6.46±0.01, about 6.53±0.01, about 6.60±0.00, about 6.67±0.00, and about 6.73±0.01. In certain embodiments, the rCFH protein is characterized by an electrophoretogram comprising percent area under the curve (AUC) values ordered from most acidic to most basic of: from 3.0% to 3.1%, from 3.6% to 4.4%, from 4.5% to 5.2%, from 5.6% to 6.5%, from 5.9% to 7.2%, from 7.1% to 8.1%, from 7.6% to 8.7%, from 8.0% to 9.1%, from 9.1% to 9.6%, from 9.4% to 10.1%, from 9.3% to 10.7%, from 7.6% to 9.5%, and from 3.3% to 6.5%, wherein the AUC values are measured by a cIEF assay. In certain embodiments, the percent AUC values ordered from most acidic to most basic are: about 3.0%±0.0%, about 3.9%±0.2%, about 4.8%±0.2%, about 5.9%±0.2%, about 6.4%±0.3%, about 7.4%±0.2%, about 8.0%±0.2%, about 8.6%±0.3%, about 9.4%±0.1%, about 9.7%±0.1%, about 10.1%±0.3%, about 8.9%±0.4%, and about 5.5%±0.8%.

In certain embodiments, the CFH protein is characterized by a free cysteine profile of trypsin-digested rCFH protein peptides of: about 0.9% of a Cys160-containing peptide fragment, about 3.2% of a Cys187-containing peptide fragment, about 3% of a Cys476-containing peptide fragment, about 1.2% of a Cys291-containing peptide fragment, about 1.2% of a Cys487-containing peptide fragment, about 0.5% of a Cys774-containing peptide fragment, about 0.6% of a Cys846-containing peptide fragment, about 0.5% of a Cys966-containing peptide fragment, about 0.5% of a Cys965-containing peptide fragment, about 0.8% of a Cys1000-containing peptide fragment, about 1% of a Cys1183-containing peptide fragment, about 1.9% of a Cys1145-containing and Cys1149-containing peptide fragment, and about 0.6% of a Cys1210-containing peptide fragment.

In certain embodiments, the CFH protein comprises less than 100 pmol of N-glycolylneuraminic (Neu5Gc) acid per mol protein, less than 10 pmol of Neu5Gc acid per mol protein, less than 1 pmol of Neu5Gc acid per mol protein, less than 0.1 pmol of Neu5Gc acid per mol protein, or less than 0.003 pmol of Neu5Gc acid per mol protein, as assayed by Ultra Performance Liquid Chromatography (UPLC).

In some embodiments, the CFH protein comprises at least CCP modules 1-4 or a biologically active fragment and/or variant thereof. As noted above, CCP modules 1-4 corresponds to amino acids 1-246 of SEQ ID NO: 1 or a variant thereof. Accordingly, in some embodiments, the CFH protein comprises an amino acid sequence that is at least 70%, 75%, 80%, 85%, 90%, 91%, 92%, 93%, 94%, 95%, 96%, 97%, 98%, 99% or 100% identical to amino acids 1-246 of SEQ ID NO: 1 (e.g., SEQ ID NO: 2, 3, 4, 5, 6, 7, 8, or 9), or a biologically active fragment thereof. In some embodiments, the CFH protein comprises an amino acid sequence that is at least 90% identical to amino acids 1-246 of SEQ ID NO: 1 (e.g., SEQ ID NO: 2, 3, 4, 5, 6, 7, 8, or 9), or a biologically active fragment thereof. In some embodiments, the CFH protein comprises an amino acid sequence that is at least 95% identical to amino acids 1-246 of SEQ ID NO: 1 (e.g., SEQ ID NO: 2, 3, 4, 5, 6, 7, 8, or 9), or a biologically active fragment thereof. In some embodiments, the CFH protein comprises amino acids 1-246 of SEQ ID NO: 1 (e.g., SEQ ID NO: 2, 3, 4, 5, 6, 7, 8, or 9), or a biologically active fragment thereof.

In some embodiments, the CFH protein comprises at least CCP modules 1-5 or a biologically active fragment and/or variant thereof. As noted above, CCP modules 1-5 corresponds to amino acids 1-304 of SEQ ID NO: 1 or a variant thereof. Accordingly, in some embodiments, the CFH protein comprises an amino acid sequence that is at least 70%, 75%, 80%, 85%, 90%, 91%, 92%, 93%, 94%, 95%, 96%, 97%, 98%, 99% or 100% identical to amino acids 1-304 of SEQ ID NO: 1 (e.g., SEQ ID NO: 2, 3, 4, 5, 6, 7, 8, or 9), or a biologically active fragment thereof. In some embodiments, the CFH protein comprises an amino acid sequence that is at least 90% identical to amino acids 1-304 of SEQ ID NO: 1 (e.g., SEQ ID NO: 2, 3, 4, 5, 6, 7, 8, or 9), or a biologically active fragment thereof. In some embodiments, the CFH protein comprises an amino acid sequence that is at least 95% identical to amino acids 1-304 of SEQ ID NO: 1 (e.g., SEQ ID NO: 2, 3, 4, 5, 6, 7, 8, or 9), or a biologically active fragment thereof. In some embodiments, the CFH protein comprises amino acids 1-304 of SEQ ID NO: 1 (e.g., SEQ ID NO: 2, 3, 4, 5, 6, 7, 8, or 9), or a biologically active fragment thereof.

In some embodiments, the CFH protein comprises at least CCP modules 1-7 or a biologically active fragment and/or variant thereof. As noted above, CCP modules 1-7 corresponds to amino acids 1-426 of SEQ ID NO: 1 or a variant thereof. Accordingly, in some embodiments, the CFH protein comprises an amino acid sequence that is at least 70%, 75%, 80%, 85%, 90%, 91%, 92%, 93%, 94%, 95%, 96%, 97%, 98%, 99% or 100% identical to amino acids 1-426 of SEQ ID NO: 1 (e.g., SEQ ID NO: 2, 3, 4, 5, 6, 7, 8, or 9), or a biologically active fragment thereof. In some embodiments, the CFH protein comprises an amino acid sequence that is at least 90% identical to amino acids 1-426 of SEQ ID NO: 1 (e.g., SEQ ID NO: 2, 3, 4, 5, 6, 7, 8, or 9), or a biologically active fragment thereof. In some embodiments, the CFH protein comprises an amino acid sequence that is at least 95% identical to amino acids 1-426 of SEQ ID NO: 1 (e.g., SEQ ID NO: 2, 3, 4, 5, 6, 7, 8, or 9), or a biologically active fragment thereof. In some embodiments, the CFH protein comprises amino acids 1-426 of SEQ ID NO: 1 (e.g., SEQ ID NO: 2, 3, 4, 5, 6, 7, 8, or 9), or a biologically active fragment thereof.

In some embodiments, the CFH protein comprises at least CCP modules 19-20 or a biologically active fragment and/or variant thereof. As noted above, CCP modules 19-20 corresponds to amino acids 1,089-1,212 of SEQ ID NO: 1 or a variant thereof. Accordingly, in some embodiments, the CFH protein comprises an amino acid sequence that is at least 70%, 75%, 80%, 85%, 90%, 91%, 92%, 93%, 94%, 95%, 96%, 97%, 98%, 99% or 100% identical to amino acids 1,089-1,212 of SEQ ID NO: 1 (e.g., SEQ ID NO: 2, 3, 4, 5, 6, 7, 8, or 9), or a biologically active fragment thereof. In some embodiments, the CFH protein comprises an amino acid sequence that is at least 90% identical to amino acids 1,089-1,212 of SEQ ID NO: 1 (e.g., SEQ ID NO: 2, 3, 4, 5, 6, 7, 8, or 9), or a biologically active fragment thereof. In some embodiments, the CFH protein comprises an amino acid sequence that is at least 95% identical to amino acids 1,089-1,212 of SEQ ID NO: 1 (e.g., SEQ ID NO: 2, 3, 4, 5, 6, 7, 8, or 9), or a biologically active fragment thereof. In some embodiments, the CFH protein comprises amino acids 1,089-1,212 of SEQ ID NO: 1 (e.g., SEQ ID NO: 2, 3, 4, 5, 6, 7, 8, or 9), or a biologically active fragment thereof.

In some embodiments, the CFH protein comprises at least CCP modules 18-20 or a biologically active fragment and/or variant thereof. As noted above, CCP modules 18-20 corresponds to amino acids 1,028-1,212 of SEQ ID NO: 1 or a variant thereof. Accordingly, in some embodiments, the CFH protein comprises an amino acid sequence that is at least 70%, 75%, 80%, 85%, 90%, 91%, 92%, 93%, 94%, 95%, 96%, 97%, 98%, 99% or 100% identical to amino acids 1,028-1,212 of SEQ ID NO: 1 (e.g., SEQ ID NO: 2, 3, 4, 5, 6, 7, 8, or 9), or a biologically active fragment thereof. In some embodiments, the CFH protein comprises an amino acid sequence that is at least 90% identical to amino acids 1,028-1,212 of SEQ ID NO: 1 (e.g., SEQ ID NO: 2, 3, 4, 5, 6, 7, 8, or 9), or a biologically active fragment thereof. In some embodiments, the CFH protein comprises an amino acid sequence that is at least 95% identical to amino acids 1,028-1,212 of SEQ ID NO: 1 (e.g., SEQ ID NO: 2, 3, 4, 5, 6, 7, 8, or 9), or a biologically active fragment thereof. In some embodiments, the CFH protein comprises amino acids 1,028-1,212 of SEQ ID NO: 1 (e.g., SEQ ID NO: 2, 3, 4, 5, 6, 7, 8, or 9), or a biologically active fragment thereof.

In some embodiments, the CFH protein is an FHL1 protein. In some embodiments, the CFH protein comprises an amino acid sequence that is at least 70%, 75%, 80%, 85%, 90%, 91%, 92%, 93%, 94%, 95%, 96%, 97%, 98%, 99% or 100% identical to the amino acid sequence of SEQ ID NO: 10 (mature FHL1 amino acid sequence), or a biologically active fragment thereof. In some embodiments, the CFH protein comprises an amino acid sequence that is at least 90% identical to the amino acid sequence of SEQ ID NO: 10, or a biologically active fragment thereof. In some embodiments, the CFH protein comprises an amino acid sequence that is at least 95% identical to the amino acid sequence of SEQ ID NO: 10, or a biologically active fragment thereof. In some embodiments, the CFH protein comprises the amino acid sequence of SEQ ID NO: 10 or a biologically active fragment thereof.

The biological activities of full-length, wild-type CFH are known in the art. It is understood that biologically active fragments and/or variants of a reference CFH sequence can be identified by functional characteristics. For example, in some embodiments, the CFH protein or biologically active fragment and/or variant thereof is capable of acting as a cofactor with CFI to facilitate C3b cleavage. In some embodiments, the CFH protein or biologically active fragment and/or variant thereof is capable of binding C3b. In some embodiments, the CFH protein or biologically active fragment and/or variant thereof is capable of facilitating the breakdown of C3b by acting in concert with complement factor I (CFI). In some embodiments, the CFH protein or biologically active fragment and/or variant thereof is capable of destabilizing C3bBb. In some embodiments, the CFH protein or biologically active fragment and/or variant thereof competes with factor B for binding to C3b. In some embodiments, the CFH protein or biologically active fragment and/or variant thereof prevents the formation of a C3 convertase (e.g. C3bBb). In some embodiments, the CFH protein or biologically active fragment and/or variant thereof accelerates the decay of convertase complexes (e.g. C3bBb). In some embodiments, the CFH protein or biologically active fragment and/or variant thereof accelerates the decay of the alternative pathway C5 convertase (C3bBbC3b). In some embodiments, the CFH protein or biologically active fragment and/or variant thereof is capable of suppressing C3b amplification. In some embodiments, the CFH protein or biologically active fragment and/or variant thereof is capable of binding to a eukaryotic cell surface (e.g., an erythrocyte and/or endothelial cell). In some embodiments, the CFH polypeptide or biologically active fragment and/or variant thereof is capable of binding to heparin. In some embodiments, the CFH protein or biologically active fragment and/or variant thereof is capable of reducing C5b9 levels generated as a result of complement activation (e.g., as measured in a Wieslab AP assay. In some embodiments, the CFH protein or biologically active and/or variant fragment thereof is capable of inhibiting hemolysis.

In some embodiments, the biologically active fragment and/or variant is at least 200, 250, 300, 350, 400, 450, 500, 600, 700, 800, 900, 1000, 1100, 1200, or 1213 amino acids in length. In some embodiments, the biologically active fragment and/or variant is between 100-1213, 200-1213, 300-1213, 400-1213, 500-1213, 600-1213, 700-1213, 800-1213, 900-1213, 1000-1213, 1100-1213, 500-1213, 100-200, 100-300, 100-400, 100-500, 100-600, 100-700, 100-800, 100-900, 200-300, 200-400, 200-500, 200-600, 200-700, 200-800, 200-900, 300-400, 300-500, 300-600, 300-700, 300-800, 300-900, 400-500, 400-600, 400-700, 400-800, 400-900, 500-600, 500-700, 500-800, 500-900, 600-700, 600-800, 600-900, 700-800, 700-900, or 800-900 amino acids in length. In some embodiments, the biologically active fragment comprises at least 100, 150, 200, 250, 300, 350, 400, 450, 500, 550, 600, 650, 700, 750, 800, 850, 900, 950, 1000, 1050, 1100, 1150, or 1200 consecutive amino acids from a sequence that is at least 70%, 75%, 80%, 85%, 90%, 91%, 92%, 93%, 94%, 95%, 96%, 97%, 98%, 99% or 100% identical to the amino acid sequence of SEQ ID NO: 1. In some embodiments, the biologically active fragment and/or variant comprises at least 100, 150, 200, 250, 300, 350, 400, 450, 500, 550, 600, 650, 700, 750, 800, 850, 900, 950, 1000, 1050, 1100, 1150, or 1200 consecutive amino acids of SEQ ID NO: 1.

In some embodiments, any of the CFH polypeptides or biologically active fragments and/or variants disclosed herein is a modified CFH polypeptide or biologically active fragment thereof as compared to a reference CFH sequence (e.g., a protein comprising the amino acid sequence of any one of SEQ ID NOs: 1-10). In some embodiments, a modified CFH polypeptide may comprise 1, 2, 3, 4, 5, up to 10, or more amino acid substitutions and/or deletions and/or insertions. A “deletion” may comprise the deletion of individual amino acids, deletion of small groups of amino acids such as 2, 3, 4 or 5 amino acids, or deletion of larger amino acid regions, such as the deletion of specific amino acid domains (e.g., one or more CCP domains) or other features. An “insertion” may comprise the insertion of individual amino acids, insertion of small groups of amino acids such as 2, 3, 4 or 5 amino acids, or insertion of larger amino acid regions, such as the insertion of specific amino acid domains or other features (e.g., insertion of a linker). A “substitution” comprises replacing a wild type amino acid with another (e.g., a non-wild type amino acid). In some embodiments, the another (e.g., non-wild type) or inserted amino acid is Ala (A), His (H), Lys (K), Phe (F), Met (M), Thr (T), Gln (Q), Asp (D), or Glu (E). In some embodiments, the another (e.g., non-wild type) or inserted amino acid is A. In some embodiments, the another (e.g., non-wild type) amino acid is Arg (R), Asn (N), Cys (C), Gly (G), Ile (I), Leu (L), Pro (P), Ser (S), Trp (W), Tyr (Y), or Val (V). Conventional or naturally occurring amino acids are divided into the following basic groups based on common side-chain properties: (1) non-polar: Norleucine, Met, Ala, Val, Leu, He; (2) polar without charge: Cys, Ser, Thr, Asn, Gin; (3) acidic (negatively charged): Asp, Glu; (4) basic (positively charged): Lys, Arg; and (5) residues that influence chain orientation: Gly, Pro; and (6) aromatic: Trp, Tyr, Phe, His. Conventional amino acids include L or D stereochemistry. In some embodiments, the another (e.g., non-wild type) amino acid is a member of a different group (e.g., an aromatic amino acid is substituted for a non-polar amino acid). Substantial modifications in the biological properties of the polypeptide are accomplished by selecting substitutions that differ significantly in their effect on maintaining (a) the structure of the polypeptide backbone in the area of the substitution, for example, as a β-sheet or helical conformation, (b) the charge or hydrophobicity of the molecule at the target site, or (c) the bulk of the side chain. Naturally occurring residues are divided into groups based on common side-chain properties: (1) Non-polar: Norleucine, Met, Ala, Val, Leu, Ile; (2) Polar without charge: Cys, Ser, Thr, Asn, Gln; (3) Acidic (negatively charged): Asp, Glu; (4) Basic (positively charged): Lys, Arg; (5) Residues that influence chain orientation: Gly, Pro; and (6) Aromatic: Trp, Tyr, Phe, His. In some embodiments, the another (e.g., non-wild type) amino acid is a member of a different group (e.g., a hydrophobic amino acid for a hydrophilic amino acid, a charged amino acid for a neutral amino acid, an acidic amino acid for a basic amino acid, etc.). In some embodiments, the another (e.g., non-wild type) amino acid is a member of the same group (e.g., another basic amino acid, another acidic amino acid, another neutral amino acid, another charged amino acid, another hydrophilic amino acid, another hydrophobic amino acid, another polar amino acid, another aromatic amino acid or another aliphatic amino acid). In some embodiments, the another (e.g., non-wild type) amino acid is an unconventional amino acid. Unconventional amino acids are non-naturally occurring amino acids. Examples of an unconventional amino acid include, but are not limited to, aminoadipic acid, beta-alanine, beta-aminopropionic acid, aminobutyric acid, piperidinic acid, aminocaprioic acid, aminoheptanoic acid, aminoisobutyric acid, aminopimelic acid, citrulline, diaminobutyric acid, desmosine, diaminopimelic acid, diaminopropionic acid, N-ethylglycine, N-ethylaspargine, hyroxylysine, allo-hydroxylysine, hydroxyproline, isodesmosine, allo-isoleucine, N-methylglycine, sarcosine, N-methylisoleucine, N-methylvaline, norvaline, norleucine, orithine, 4-hydroxyproline, γ-carboxyglutamate, ϵ-N,N,N-trimethyllysine, ϵ-N-acetyllysine, O-phosphoserine, N-acetylserine, N-formylmethionine, 3-methylhistidine, 5-hydroxylysine, σ-N-methylarginine, and other similar amino acids and amino acids (e.g., 4-hydroxyproline). In one aspect, a modified CFH protein or biologically active fragment thereof comprises 1, 2, 3, 4, 5, 6, 7, 8, 9, 10, 11, 12, 13, 14, or 15 conservative or non-conservative substitutions relative to the wild-type CFH polypeptide or biologically active fragment thereof (e.g., a polypeptide having the amino acid sequence of any one of SEQ ID NOs: 1-10).

In certain embodiments, any of the CFH polypeptides or biologically active fragments and/or variants disclosed herein may further comprise post-translational modifications in addition to any that are naturally present in the native polypeptides. Such modifications include, but are not limited to, acetylation, carboxylation, glycosylation, phosphorylation, lipidation, pegylation (polyethylene glycol) and acylation. As a result, the modified polypeptides may contain non-amino acid elements, such as polyethylene glycols, lipids, mono- or poly-saccharides, and phosphates. Effects of such non-amino acid elements on the functionality of a polypeptide may be tested as described herein for other polypeptide variants. When a polypeptide is produced in cells by cleaving a nascent form of the polypeptide, post-translational processing may also be important for correct folding and/or function of the protein. Different cells have specific cellular machinery and characteristic mechanisms for such post-translational activities and may be chosen to ensure the correct modification and processing of the polypeptides.

### CFH-Encoding Vectors

The present disclosure also provides a nucleic acid encoding CFH protein, including a biologically active fragment and/or variant of a full-length, wild-type CFH protein, for use in any of the methods disclosed herein. In certain embodiments, the nucleic acid is present in a vector (e.g., a viral vector), which can be formulated in a pharmaceutical composition and administered to a subject in need thereof.

In certain embodiments, the vector comprises a nucleotide sequence encoding a CFH protein disclosed herein operably linked to an expression control sequence. As used herein, the term “expression control sequence” refers to a nucleotide sequence that directs transcription and/or translation of a nucleic acid. Expression control sequences include but are not limited to transcription initiation, termination, promoter and enhancer sequences; RNA processing signals such as splicing and polyadenylation (polyA) signals; sequences that stabilize cytoplasmic mRNA; sequences that enhance translation efficiency (e.g., Kozak consensus sequence); and sequences that enhance protein stability. In certain embodiments, the vector comprises a promoter and/or an enhancer operably linked to the CFH-coding sequence.

In certain embodiments, the vector comprises a constitutive promoter operably linked to the CFH-coding sequence. In certain embodiments, the constitutive promoter is a CMV promoter (optionally with the CMV enhancer), RSV promoter (optionally with the RSV enhancer), SV40 promoter, MoMLV promoter, CB promoter, the dihydrofolate reductase promoter, the chicken R-actin (CBA) promoter, CBA/CAG promoter, and the immediate early CMV enhancer coupled with the CBA promoter, or a EF1a promoter.

In other embodiments, the vector comprises a cell- or tissue-specific expression control sequence, for example, a promoter and/or an enhancer that can direct expression of CFH preferentially in one or more ocular cell types, such as a rod cell, cone cell, bipolar cell (e.g., on-bipolar cell, off-bipolar cell), horizontal cell, amacrine cell, retinal pigment epithelium (RPE) cell, retinal glial cell (e.g., Muller cell), and/or retinal ganglion cell. In certain embodiments, the expression control sequence comprises a metabotropic glutamate receptor 6 (mGluR6) promoter (see, Vardi et al, mGluR6 Transcripts in Non-neuronal Tissues, J Histochem Cytochem. 2011 December; 59(12): 1076-1086). In certain embodiment, the expression control sequence further comprises an mGluR6 enhancer. In certain embodiment, the expression control sequence comprises a human G-protein-coupled receptor protein kinase 1 (GRK1) promoter (Genbank Accession number AY327580). In certain embodiment, the expression control sequence comprises a human interphotoreceptor retinoid-binding protein proximal (IRBP) promoter. Other useful promoters include, without limitation, the rod opsin promoter, the red-green opsin promoter, the blue opsin promoter, the cGMP-β-phosphodiesterase promoter, the mouse opsin promoter (Beltran et al 2010 cited above), the rhodopsin promoter (Mussolino et al, Gene Ther, July 2011, 18(7):637-45); the alpha-subunit of cone transducin (Morrissey et al, BMC Dev, Biol, January 2011, 11:3); beta phosphodiesterase (PDE) promoter; the retinitis pigmentosa (RP1) promoter (Nicoud et al, J. Gene Med, December 2007, 9(12): 1015-23); the NXNL2/NXNL1 promoter (Lambard et al, PLoS One, October 2010, 5(10):e13025), the RPE65 promoter; the retinal degeneration slow/peripherin 2 (Rds/perph2) promoter (Cai et al, Exp Eye Res. 2010 August; 91(2): 186-94); and the VMD2 promoter (Kachi et al, Human Gene Therapy, 2009 (20:31-9)).

In certain embodiments, the expression control sequence is the native promoter for the gene to be expressed (e.g., the CFH gene or the FHL-1 gene).

In certain embodiments, the expression control sequence comprises an enhancer. Enhancer sequences useful herein include but are not limited to the IRBP enhancer (Nicoud et al, J. Gene Med, December 2007, 9(12): 1015-23), immediate early CMV enhancer, immunoglobulin enhancer, and SV40 enhancer.

Other expression control sequences useful herein include an intron, optionally positioned between the promoter/enhancer sequence and the gene. In some embodiments, the intron sequence is derived from SV-40, and is a 100 bp mini-intron splice donor/splice acceptor referred to as SD-SA. Another suitable sequence includes the woodchuck hepatitis virus post-transcriptional element (see, e.g., L. Wang and I. Verma, 1999 Proc. Natl. Acad. Sci., USA, 96:3906-3910). PolyA signals may be derived from many suitable species, including, without limitation SV-40, human and bovine.

Another expression control sequence useful herein is an internal ribosome entry site (IRES). An IRES sequence may be used to produce more than one polypeptide from a single gene transcript (for example, to produce more than one complement system polypeptides). An IRES (or other suitable sequence) is used to produce a protein that contains more than one polypeptide chain or to express two different proteins from or within the same cell. An exemplary IRES is the poliovirus internal ribosome entry sequence, which supports transgene expression in photoreceptors, RPE and ganglion cells. Preferably, the IRES is located 3′ to the CFH-coding sequence in the vector.

Expression control sequences that are small in size are desirable in vectors that have a size limitation, for example, adeno-associated virus (AAV) vectors. In some embodiments, the expression control sequence is less than 1000, 900, 800, 700, 600, 500, 400 or 300 nucleotides in size. In some embodiments, the expression control sequence comprises, consists of, or consists essentially of a small promoter, e.g., a CRALBP (RLBP), EF1a, HSP70, AAT1, ALB, PCK1, CAG, RPE65, MECP, sCBA, or CB promoter.

In certain embodiments, the vector is an AAV vector, for example, a recombinant AAV (rAAV) vector. A rAAV vector comprises one or more heterologous sequences (i.e., nucleic acid sequence not of AAV origin) that are flanked by at least one AAV inverted terminal repeat sequence (ITR). Such rAAV vectors can be replicated and packaged into infectious viral particles in a host cell that has been infected with a suitable helper virus or that is expressing suitable helper genes such as AAV rep and cap. When a rAAV vector is incorporated into a larger polynucleotide (e.g., in a chromosome or in another vector such as a plasmid used for cloning or transfection), the vector is referred to as a “pro-vector” which can be used to produce a mature rAAV vector by replication and encapsidation in the presence of AAV packaging functions and suitable helper functions. An rAAV vector can be in any of a number of forms, including, but not limited to, plasmids, linear artificial chromosomes, complexed with lipids, encapsulated within liposomes, and encapsidated in a viral particle, e.g., an AAV particle. A rAAV vector can be packaged into an AAV virus capsid to generate a recombinant adeno-associated viral particle (rAAV particle).

AAV particles are present in various serotypes. Naturally occurring AAV serotypes include, without limitation, AAV1, AAV2, AAV3, AAV4, AAV5, AAV6, AAV7, AAV8, AAV9, AAV10, AAV10, AAV11, AAV12, AAV-DJ, AAV-DJ8, AAV-DJ9, AAVrh8, AAVrh8R and AAVrh10. Each serotype has its unique capsid proteins and ITR sequences that is related to its tropism. For example, AAV1, AAV2, AAV4, AAV5, AAV7 and AAV8 are suitable for gene transfer to photoreceptor cells or the retinal pigment epithelium (RPE) cells. Non-naturally occurring serotypes can also be created by engineering the ITR and/or capsid. A recombinant AAV can be chimeric, comprising the ITR sequence from one serotype and the capsid from a different serotype.

Generally, an “inverted terminal repeat” or “ITR” sequence is an approximately 145-nucleotide sequence present at both termini of the native single-stranded AAV genome. The outermost 125 nucleotides of the ITR can be present in either of two alternative orientations, leading to heterogeneity between different AAV genomes and between the two ends of a single AAV genome. The outermost 125 nucleotides also contains several shorter regions of self-complementarity (designated A, A′, B, B′, C, C and D regions), allowing intrastrand base-pairing to occur within this portion of the ITR. In certain embodiments, the AAV vector disclosed herein comprises the ITR sequences of AAV1, AAV2, AAV3, AAV4, AAV5, AAV6, AAV7, AAV8, AAV9, AAV10, AAV10, AAV11, AAV12, AAV-DJ, AAV-DJ8, AAV-DJ9, AAVrh8, AAVrh8R or AAVrh10. In certain embodiments, the AAV vector comprises the ITR sequences of AAV2.

The capsid of AAV particles includes Cap proteins VP1, VP2, and VP3. In certain embodiments, the AAV vector disclosed herein is present in an AAV particle comprising VP1, VP2, and VP3 of AAV1, AAV2, AAV3, AAV4, AAV5, AAV6, AAV7, AAV8, AAV9, AAV10, AAV10, AAV11, AAV12, AAV-DJ, AAV-DJ8, AAV-DJ9, AAVrh8, AAVrh8R or AAVrh10, or a variant or derivative thereof. In certain embodiments, the AAV particle comprises VP1, VP2, and VP3 of AAV2 or a variant or derivative thereof. In certain embodiments, the AAV capsid serotype is AAV2. In certain embodiments, the AAV capsid serotype is AAV.7m8 (see, e.g., International Patent Application Publication No. WO 2012/145601). In certain embodiments, the capsid is modified by amino acid deletion, insertion, and/or substitution to reduce immunogenicity, improve stability, reduce degradation, and/or improve the efficiency of infecting an ocular cell. It is understood that in a pseudotyped AAV particle the Cap proteins can be encoded by a nucleic acid in the producer cell but not in the AAV genome. Accordingly, in certain embodiments, the AAV particle does not comprise a nucleotide sequence encoding a Cap protein.

AAV particles contain a single-stranded genome when packaged into the capsid. However, self-complementary AAV having an inverted repeat genome can be produced (see, US 2012/0141422). Such AAV genome can fold into double-stranded DNA without the requirement for DNA synthesis or base-pairing between multiple vector genomes. Because scAAV have no need to convert the single-stranded DNA genome into double-stranded DNA prior to expression, they are more efficient vectors. However, self-complementary AAV genomes has only half of the capacity of the vector relative to single-stranded AAV genomes and are thus useful for small transgenes (e.g., small protein-coding sequences and RNA-based therapies). In some embodiments, the rAAV vector disclosed herein comprises a single-stranded genome. In some embodiments, the rAAV vector disclosed herein comprises a self-complementary genome.

rAAV vectors useful in the methods of the disclosure are further described in PCT publication No. WO2015168666 and PCT publication no. WO2014011210, the contents of which are incorporated by reference herein.

Recombinant AAV particles can be produced by delivering the recombinant AAV vector, rep sequences, cap sequences, and helper functions to a packaging host cell. Methods of generating rAAV virions are well known in the art (see, e.g., K. Fisher et al, J. Virol., 70:520-532 (1993) and U.S. Pat. No. 5,478,745). In some embodiments, recombinant AAVs may be produced using the triple transfection method (described in detail in U.S. Pat. No. 6,001,650). Typically, the recombinant AAVs are produced by transfecting a host cell with an recombinant AAV vector (comprising a transgene) to be packaged into AAV particles, an AAV helper function vector, and an accessory function vector. An AAV helper function vector encodes the “AAV helper function” sequences (e.g., rep and cap), which function in trans for productive AAV replication and encapsidation. In some embodiments, a single nucleic acid encoding all three capsid proteins (e.g., VP1, VP2 and VP3) is delivered into the packaging host cell in a single vector. The accessory functions are conferred by nucleotide sequences for non-AAV derived viral and/or cellular functions upon which AAV is dependent for replication. In certain embodiments, the accessory function vector comprises viral genetic elements, for example, elements derived from any of the known helper viruses such as adenovirus, herpesvirus (other than herpes simplex virus type-1), and vaccinia virus. In certain embodiments, the accessory function vector comprises genetic elements from an adenovirus, e.g., E1a, E1b, E2a, and/or E40RF6. One or more of the required components for producing a rAAV may be provided by a host cell which has been engineered to contain the components in its genome.

### Pharmaceutical Compositions

Also provided herein are pharmaceutical compositions comprising a CFH protein disclosed herein and a pharmaceutically acceptable carrier or excipient for use in the method disclosed herein. The pharmaceutical compositions may be suitable for any mode of administration described herein; for example, by ocular (e.g., intravitreal) administration. In some embodiments, the pharmaceutical compositions described herein is suitable for ocular injection. In some embodiments, the pharmaceutical composition is suitable for intravitreal injection. In some embodiments, the pharmaceutical composition is suitable for subretinal delivery.

Pharmaceutically acceptable carriers and excipients suitable for administration to a subject (e.g., human subject) are well known in the art (see, e.g., Remington's Pharmaceutical Sciences, 15th Edition, pp. 1035-1038 and 1570-1580). Such pharmaceutically acceptable carriers can be sterile liquids, such as oil, including those of petroleum, animal, vegetable or synthetic origin, such as peanut oil, soybean oil, mineral oil, and the like. Saline solutions and aqueous dextrose, polyethylene glycol (PEG) and glycerol solutions can also be employed as liquid carriers, particularly for injectable solutions. The pharmaceutical composition may further comprise additional ingredients, for example preservatives, buffers, tonicity agents, antioxidants and stabilizers, nonionic wetting or clarifying agents, viscosity-increasing agents, and the like. The pharmaceutical compositions described herein can be packaged in single unit dosages or in multi-dosage forms. The compositions are generally formulated as sterile and substantially isotonic solution.

The pharmaceutical composition may further comprise additional ingredients, for example preservatives, buffers, tonicity agents, antioxidants and stabilizers, nonionic wetting or clarifying agents, viscosity increasing agents (viscosity inducing agents), and the like. Examples of useful viscosity-inducing components include, but are not limited to, hyaluronic acid (e.g., polymeric hyaluronic acid), carbomer, polyacrylic acid, cellulose derivatives, polycarbophil, polyvinyl pyrrolidone, gelatin, dextrin, polysaccharide, polyacrylamide, polyvinyl alcohol, polyvinyl acetate, derivatives thereof and mixtures and copolymers thereof.

The composition is generally formulated as a sterile and substantially isotonic solution. In some embodiments, the compositions of the present disclosure are formulated into a pharmaceutical composition intended for subretinal or intravitreal injection. Such formulations involve the use of a pharmaceutically and/or physiologically acceptable vehicle or carrier, particularly one suitable for administration to the eye, e.g., by subretinal or intravitreal injection, such as buffered saline or other buffers, e.g., HEPES, to maintain pH at appropriate physiological levels, and, optionally, other medicinal agents, pharmaceutical agents, stabilizing agents, buffers, carriers, adjuvants, and/or diluents. For injection, the carrier will typically be a liquid. Exemplary physiologically acceptable carriers include pyrogen-free, phosphate buffered saline. A variety of such known carriers are provided in U.S. Pat. No. 7,629,322. In some embodiments, the carrier is an isotonic sodium chloride solution. In some embodiments, the isotonic sodium chloride solution comprises about 0.15 M sodium chloride. In another embodiment, the carrier is a balanced salt solution. In one embodiment, the carrier includes a surfactant such as TWEEN®20 or perfluorooctane (Perfluoron liquid). If the CFH polypeptide or biologically active fragment and/or variant thereof is to be stored long-term, it may be frozen in the presence of glycerol or TWEEN®20.

In some embodiments, the pharmaceutical composition is substantially non-toxic, for example, to intraocular tissues. When the compositions include a liquid carrier and are formulated for ocular administration, it is advantageous that the carrier component has a refractive index substantially similar to that of aqueous humor or vitreous humor (depending on the chamber into which the formulation is introduced). As a result, there is no substantial adverse effect on the patient's vision, for example, by changing the focus after administration. Formulations with a refractive index of water (approximately 1.33 depending on the wavelength of light) can adversely affect the patient's vision at the time after administration, for example, at the boundary between the injected formulation and the vitreous humor after injection.

The pharmaceutical composition can be formulated at pH levels that are suitable for direct administration to the eye (e.g., intravitreal injection). Generally, pH values that are suitable for safe intravitreal injection range from 3-8 (see, for example, Marra et al., AAPS PharmSciTech, 2011) or 4.0-7.4 (see, for example, Lorget et al., Investigative Ophthalmology & Visual Science, 2018). In some embodiments, the pharmaceutical composition disclosed herein formulated to maintain a physiological pH (e.g., a pH of about 7). In some embodiments, the pH of the pharmaceutical composition is in the range of about 5.0 to 8.0, about 5.0 to 7.0, about 5.0 to 6.0, about 6.0 to 8.0, about 6.1 to 8.0, about 6.2 to 8.0, about 6.3 to 8.0, about 6.4 to 8.0, about 6.5 to 8.0, about 6.6 to 8.0, about 6.7 to 8.0, about 6.8 to 8.0, about 6.9 to 8.0, about 7.0 to 8.0, about 7.1 to 8.0, about 7.2 to 8.0, about 7.3 to 8.0, about 7.4 to 8.0, about 7.5 to 8.0, about 7.6 to 8.0, about 7.7 to 8.0, about 7.8 to 8.0, about 7.9 to 8.0, about 6.60 to 7.40, about 6.0 to 7.5, about 6.1 to 7.5, about 6.2 to 7.5, about 6.3 to 7.5, about 6.4 to 7.5, about 6.5 to 7.5, about 6.6 to 7.5, about 6.7 to 7.5, about 6.8 to 7.5, about 6.9 to 7.5, about 7.0 to 7.5, about 7.1 to 7.5, about 7.2 to 7.5, about 7.3 to 7.5, about 7.4 to 7.5, about 6 to 7.0, about 6.1 to 7.0, about 6.2 to 7.0, about 6.3 to 7.0, about 6.4 to 7.0, about 6.5 to 7.0, about 6.6 to 7.0, about 6.7 to 7.0, about 6.8 to 7.0, about 6.9 to 7.0. In some embodiments, the pH of the pharmaceutical composition is about 5.0, about 5.5, about 6.0, about 6.1, about 6.2, about 6.3, about 6.4, about 6.5, about 6.6, about 6.7, about 6.8, about 6.9, about 7.0, about 7.1, about 7.2, about 7.3, about 7.4, about 7.5, about 7.6, about 7.7, about 7.8, about 7.9, or about 8.0.

In some embodiments, the pharmaceutical composition disclosed herein comprise buffering agents such as sodium phosphate monobasic monohydrate and/or sodium phosphate dibasic heptahydrate. These buffering agents are used to maintain the pH of the compositions. Other buffering agents are contemplated in the compositions of the present disclosure. Alternative buffer components may be selected from those known in the art. Non-limiting examples of buffers include acetate buffer, citrate buffer, phosphate buffer, borate buffer, and the like, and mixtures thereof.

In some embodiments, the pharmaceutical composition disclosed herein comprise a stabilizer such as sodium chloride and/or polysorbate 20. In addition to stabilizing the compositions, polysorbate 20 prevents the formation of protein aggregates (e.g., due to agitation) in the composition. Polysorbate 20 protects against agitation-induced stress on the CFH protein in solution. Other stabilizers are contemplated in the compositions of the present disclosure.

In some embodiments, the pharmaceutical composition disclosed herein is formulated to be isotonic. This can be achieved using a tonicity agent. In some embodiments, the compositions of the present disclosure comprise a tonicity agent, such as sodium chloride. Other tonicity agents are contemplated in the compositions of the present disclosure. Non-limiting examples of tonicity agents include glycerin, lactose, mannitol, dextrose, sodium chloride, sodium sulfate, sorbitol, physiological saline-sodium citrate (SSC), and the like.

The concentration of the active ingredient (e.g., a CFH protein) in the pharmaceutical composition can be determined based on the desired dose and the volume clinically suitable for intravitreal administration. In certain embodiments, the pharmaceutical composition comprises CFH protein in range of 1-50 mg/mL (e.g., 1-20 mg/mL, 1-10 mg/mL, 5-50 mg/mL, 5-20 mg/mL, 5-10 mg/mL, or 10-20 mg/mL). In certain embodiments, the pharmaceutical composition comprises CFH protein in range of 9.0-11.0 mg/mL. In some embodiments, the concentration of CFH protein in the pharmaceutical composition is about 9.0 mg/mL, about 9.1 mg/mL, about 9.2 mg/mL, about 9.3 mg/mL, about 9.4 mg/mL, about 9.5 mg/mL, about 9.6 mg/mL, about 9.7 mg/mL, about 9.8 mg/mL, about 9.9 mg/mL, about 10 mg/mL, about 10.1 mg/mL, about 10.2 mg/mL, about 10.3 mg/mL, about 10.4 mg/mL, about 10.5 mg/mL, about 10.6 mg/mL, about 10.7 mg/mL, about 10.8 mg/mL, about 10.9 mg/mL, or about 11 mg/mL. In some embodiments, the concentration of CFH protein in the pharmaceutical composition is in the range of 9.0-11.0 mg/mL, about 9.0-10.5 mg/mL, about 9.0-10.0 mg/mL, about 9.0-9.5 mg/mL, about 9.5-11.0 mg/mL, about 9.-5-10.5 mg/mL, about 9.5-10.0 mg/mL, about 10.0-11.0 mg/mL, about 10.0-10.5 mg/mL, or about 10.5-11.0 mg/mL.

In certain embodiments, the pharmaceutical composition used in the method of treatment disclosed herein comprises phosphate-buffer saline (PBS). In certain embodiments, the pharmaceutical composition comprises a surfactant. In certain embodiments, the pharmaceutical composition comprises PBS and a surfactant. In certain embodiments, the CFH protein is administered by intravitreal injection in a solution that comprises 0.02 M sodium phosphate and 0.15 M NaCl. In certain embodiments, the solution further comprises from 0.01% to 0.05% by weight of a surfactant. In certain embodiments, the solution further comprises from 0.010% to 0.05% by weight polysorbate 20.

In some embodiments, the pharmaceutical composition comprises 5-20 mg/mL (e.g., about 10 mg/mL) CFH protein and one or more excipients selected from sodium phosphate monobasic monohydrate, sodium phosphate dibasic heptahydrate, sodium chloride, and/or polysorbate 20. In some embodiments, the pharmaceutical compositions include water. In some embodiments, the pharmaceutical composition comprises about 0.02 M sodium phosphate, about 0.15 M sodium chloride, and about 0.01% (w/v) polysorbate 20, at pH 7.0. In some embodiments, the pharmaceutical composition comprises 0.02 M sodium phosphate, 0.15 M sodium chloride, and 0.01% (w/v) polysorbate 20, at pH 7.0.

In some embodiments, the pharmaceutical composition consists essentially of CFH protein and one or more excipients selected from sodium phosphate monobasic monohydrate, sodium phosphate dibasic heptahydrate, sodium chloride, and polysorbate 20. In some embodiments, the pharmaceutical composition consists essentially of CFH protein and about 0.02 M sodium phosphate, about 0.15 M sodium chloride, and about 0.01% (w/v) polysorbate 20, at pH 7.0. In some embodiments, the pharmaceutical composition consists essentially of 0.02 M sodium phosphate, 0.15 M sodium chloride, and 0.01% (w/v) polysorbate 20, at pH 7.0.

### Route of Administration

As used herein, “administering” or “administration” of a substance, a compound or an agent to a subject can be carried out using one of a variety of methods known to those skilled in the art. For example, a compound or an agent can be administered intravitreally or subretinally. In particular embodiments, the compound or agent is administered intravitreally. In some embodiments, administration may be local. In other embodiments, administration may be systemic. Administering can also be performed, for example, once, a plurality of times, and/or over one or more extended periods. In some aspects, the administration includes both direct administration, including self-administration, and indirect administration, including the act of prescribing a drug. For example, as used herein, a physician who instructs a patient to self-administer a drug, or to have the drug administered by another and/or who provides a patient with a prescription for a drug is administering the drug to the patient.

In some embodiments, the CFH protein or pharmaceutical composition disclosed herein is administered locally to the cells in the retina for treating diseases such as LCA, retinitis pigmentosa, and age-related macular degeneration. The cells that will be the treatment target in these diseases are either the photoreceptor cells in the retina or the cells of the RPE underlying the neurosensory retina. Delivering the CFH protein or pharmaceutical composition to these cells may be by injection into the subretinal space between the retina and the RPE. In some embodiments, the disclosure provides methods to deliver the CFH protein or pharmaceutical composition to cells of the retina.

In some embodiments, any of the CFH protein or pharmaceutical composition disclosed herein are administered to a patient such that they target cells of any one or more layers or regions of the retina or macula. For example, the compositions disclosed herein target cells of any one or more layers of the retina, including the inner limiting membrane, the nerve fiber layer, the ganglion cell layer (GCL), the inner plexiform layer, the inner nuclear layer, the outer plexiform layer, the outer nuclear layer, the external limiting membrane, the layer of rods and cones, or the retinal pigment epithelium (RPE). In some embodiments, the compositions disclosed herein target glial cells of the GCL, Muller cells, and/or retinal pigment epithelial cells. In some embodiments, the compositions disclosed herein targets cells of any one or more regions of the macula including, for example, the umbo, the foveolar, the foveal avascular zone, the fovea, the parafovea, or the perifovea. In some embodiments, the route of administration does not specifically target neurons. In some embodiments, the route of administration is chosen such that it reduces the risk of retinal detachment in the patient (e.g., intravitreal rather than subretinal administration).

In certain embodiments of the methods described herein, the CFH or pharmaceutical composition disclosed herein is administered to the subject by subretinal injection. In other embodiments, the pharmaceutical composition is administered by intravitreal injection. Other forms of administration that may be useful in the methods described herein include, but are not limited to, direct delivery to a desired organ (e.g., the eye), oral, inhalation, intranasal, intratracheal, intravenous, intramuscular, subcutaneous, intradermal, and other parenteral routes of administration. Routes of administration may be combined, if desired. In certain embodiments, the pharmaceutical compositions of the disclosure are administered after administration of an initial loading dose of the CFH polypeptide or biologically active fragment and/or variant thereof.

In specific embodiments, the CFH or pharmaceutical composition disclosed herein is administered to the subject by intravitreal (IVT) injection. Procedures for intravitreal injection are known in the art (see, e.g., Peyman, G. A., et al. (2009) Retina 29(7):875-912 and Fagan, X. J. and Al-Qureshi, S. (2013) Clin. Experiment. Ophthalmol. 41(5):500-7). Briefly, a subject for intravitreal injection may be prepared for the procedure by pupillary dilation, sterilization of the eye, and administration of anesthetic. Any suitable mydriatic agent known in the art may be used for pupillary dilation. Adequate pupillary dilation may be confirmed before treatment. Sterilization may be achieved by applying a sterilizing eye treatment, e.g., an iodide-containing solution such as Povidone-Iodine (BETADINE®). A similar solution may also be used to clean the eyelid, eyelashes, and any other nearby tissues (e.g., skin). Any suitable anesthetic may be used, such as lidocaine or proparacaine, at any suitable concentration. Anesthetic may be administered by any method known in the art, including without limitation topical drops, gels or jellies, and subconjuctival application of anesthetic. Prior to injection, a sterilized eyelid speculum may be used to clear the eyelashes from the area. The site of the injection may be marked with a syringe. The site of the injection may be chosen based on the lens of the patient. For example, the injection site may be 3-3.5 mm from the limbus in pseudophakic or aphakic patients, and 3.5-4 mm from the limbus in phakic patients. The patient may look in a direction opposite the injection site. During injection, the needle may be inserted perpendicular to the sclera and pointed to the center of the eye. The needle may be inserted such that the tip ends in the vitreous, rather than the subretinal space. Any suitable volume known in the art for injection may be used. After injection, the eye may be treated with a sterilizing agent such as an antibiotic. The eye may also be rinsed to remove excess sterilizing agent. Ophthalmologists are accustomed to providing frequent (e.g., monthly) IVT injections for wet macular degeneration. It is not uncommon for patients to be treated on a regular basis for long periods of time (e.g., years). The risk of severe complications following IVT injection such as uveitis, retinal detachment, extensive IVT hemorrhage, raised intraocular pressure (IOP), or endophthalmitis.

Furthermore, in certain embodiments, it is desirable to perform non-invasive retinal imaging and functional studies to identify areas of specific ocular cells to be targeted for therapy. In these embodiments, clinical diagnostic tests are employed to determine the precise location(s) for one or more subretinal injection(s). These tests may include ophthalmoscopy, electroretinography (ERG) (particularly the b-wave measurement), perimetry, topographical mapping of the layers of the retina and measurement of the thickness of its layers by means of confocal scanning laser ophthalmoscopy (cSLO) and optical coherence tomography (OCT), topographical mapping of cone density via adaptive optics (AO), functional eye exam, etc.

These, and other desirable tests, are described in International Patent Application No. PCT/US2013/022628. In view of the imaging and functional studies, in some embodiments, one or more injections are performed in the same eye in order to target different areas of retained bipolar cells. The volume and concentration of the CFH polypeptide or biologically active fragment and/or variant thereof for each injection is determined individually, as further described below, and may be the same or different from other injections performed in the same, or contralateral, eye. In another embodiment, a single, larger volume injection is made in order to treat the entire eye. In one embodiment, the volume and concentration of the CFH polypeptide or biologically active fragment and/or variant thereof composition is selected so that only a specific region of ocular cells is impacted. In another embodiment, the volume and/or concentration of the CFH polypeptide or biologically active fragment and/or variant thereof composition is a greater amount, in order reach larger portions of the eye, including non-damaged ocular cells.

The composition may be delivered in a volume of from about 0.1 μL to about 1 mL, including all numbers within the range, depending on the size of the area to be treated, the route of administration, and the desired effect of the method. In certain embodiments, the volume is between 10-100 μL. In some embodiments, the volume is between 25-100 μL. In some embodiments, the volume is between 40-60 μL. In one embodiment, the volume is about 50 μL. In one embodiment, the volume is 50 μL. In another embodiment, the volume is about 70 μL. In a preferred embodiment, the volume is about 100 μL. In another embodiment, the volume is about 125 μL. In another embodiment, the volume is about 150 μL. In another embodiment, the volume is about 175 μL. In yet another embodiment, the volume is about 200 μL. In another embodiment, the volume is about 250 μL. In another embodiment, the volume is about 300 μL. In another embodiment, the volume is about 450 μL. In another embodiment, the volume is about 500 μL. In another embodiment, the volume is about 600 μL. In another embodiment, the volume is about 750 μL. In another embodiment, the volume is about 850 μL. In another embodiment, the volume is about 1000 μL.

It is desirable that the lowest effective concentration of CFH protein be utilized in order to reduce the risk of undesirable effects, such as toxicity, retinal dysplasia and detachment. Still other dosages and administration volumes in these ranges may be selected by the attending physician, taking into account the physical state of the subject, preferably human, being treated, the age of the subject, the particular ocular disorder and the degree to which the disorder, if progressive, has developed. For extra-ocular delivery, the dosage will be increased according to the scale-up from the retina.

### Combination Therapy

In certain embodiment, the method disclosed herein is performed in combination with another, or secondary, therapy. The therapy may be any now known, or as yet unknown, therapy which helps prevent, arrest or ameliorate any of the described retinal changes and/or vision loss. In one embodiment, the secondary therapy is encapsulated cell therapy (such as that delivering Ciliary Neurotrophic Factor (CNTF)). See, e.g., Sieving, P. A. et al, 2006. Proc Natl Acad Sci USA, 103(10):3896-3901, which is hereby incorporated by reference. In another embodiment, the secondary therapy is a neurotrophic factor therapy (such as pigment epithelium-derived factor, PEDF; ciliary neurotrophic factor 3; rod-derived cone viability factor (RdCVF) or glial-derived neurotrophic factor). In another embodiment, the secondary therapy is anti-apoptosis therapy (such as that delivering X-linked inhibitor of apoptosis, XIAP). In yet another embodiment, the secondary therapy is rod-derived cone viability factor 2. The secondary therapy can be administered before, concurrent with, or after administration of any of the CFH polypeptides or biologically active fragments and/or variants thereof described above.

In some embodiments, the CFH or pharmaceutical composition disclosed herein is administered to a subject in combination with another therapeutic agent or therapeutic procedure. In some embodiments, the additional therapeutic agent is an antagonist of VEGF (e.g., VEGF-A), such as an anti-VEGF antibody or fragment thereof (e.g., ranibizumab or bevacizumab) or a decoy protein (e.g., aflibercept). VEGF antagonists and their uses for treating ocular diseases were reviewed by Fogli et al., Eye (2018) 32, 1010-20. In some embodiments, the additional therapeutic agent is a vitamin or mineral (e.g., vitamin C, vitamin E, lutein, zeaxanthin, zinc or copper), omega-3 fatty acids, and/or Visudyne™. In some embodiments, the inflammatory ocular disease, disorder, or condition is wet AMD or diabetic macular edema, and the CFH or pharmaceutical composition disclosed herein is administered to the subject in combination with an anti-VEGF therapeutic agent. In some embodiments, the other therapeutic procedure is a diet having reduced omega-6 fatty acids, laser surgery, laser photocoagulation, submacular surgery, retinal translocation, and/or photodynamic therapy. In some embodiments, the additional therapeutic agent is a vector (e.g., an AAV vector) encoding a CFH protein or biologically active fragment/variant thereof and/or a CFI protein or a biologically active fragment/variant thereof.

In some embodiments, the CFH or pharmaceutical composition disclosed herein is administered to a subject in combination with aflibercept. In some embodiments, aflibercept is administered at the dose of 2 mg, 4 mg, or 8 mg. In some embodiments, aflibercept is administered at the dose of 2 mg once every month, once every 4 weeks, once every two months, or once every 8 weeks. In some embodiments, aflibercept is administered at the dose of 8 mg once every two months, once every 8 weeks, once every three months, once every 12 weeks, once every four months, or once every 16 weeks.

In some embodiments, the CFH or pharmaceutical composition disclosed herein is administered to a subject in combination with an additional agent needed for processing and/or improving the function of CFH. For example, the CFH or pharmaceutical composition disclosed herein may be administered with an antibody (or a vector encoding that antibody) that potentiates the activity of the administered CFH and an endogenous CFH protein. Examples of such antibodies are found in WO2016/028150 or WO2019/139481.

It is understood that the CFH or pharmaceutical composition disclosed herein can be administered separately, optionally by a different route and/or at a different frequency, from the therapy used in combination.

### Therapeutic Efficacy

The efficacy of the treatment can be determined by performing functional and imaging studies to the subject. These studies include ERG and in vivo retinal imaging, as described in the examples below. In addition, visual field studies, perimetry and microperimetry, pupillometry, mobility testing, visual acuity, contrast sensitivity, color vision testing may be performed.

In certain embodiments, the progression or severity of the inflammatory ocular disease, disorder, or condition can be assessed by any suitable method known in the art, including, but not limited to best corrected visual acuity (BCVA) score, low luminance visual acuity (LLVA) score, AREDS 9-step severity scale score, area of geographic atrophy, drusen volume, or a retinal architecture parameter. In certain embodiments, the BCVA score is improved by at least 8-letter, at least 9-letter, at least 10-letter, at least 11-letter, or at least 12-letter. In certain embodiments, the LLVA score is improved by at least 4-letter, at least 5-letter, at least 6-letter, or at least 7-letter. The area of geographic atrophy can be assessed by any suitable method, including, but not limited to, color fundus photography, fundus autofluorescence optical coherence tomography-angiography, near infrared imaging, and/or fluorescein angiography. In certain embodiments, the rate of expansion in the area of geographic atrophy is reduced by at least 20%, at least 30%, at least 40%, at least 50%, at least 60%, at least 70%, at least 80%, or at least 90% relative to the rate of expansion in the same subject prior to the treatment. Retinal architecture parameters can be assessed by any suitable method including, but not limited to, optical coherence tomography. Exemplary retinal architecture parameters include total retinal and choroidal thickness, photoreceptor layer thickness, features of nascent geographic atrophy, retinal pigment epithelium thickening, and integrity of retinal pigment epithelium layer. In certain embodiments, one or more retinal architecture parameters are improved based on clinical standards known in the art.

The retinal diseases described above are associated with various retinal changes. These may include a loss of photoreceptor structure or function; thinning or thickening of the outer nuclear layer (ONL); thinning or thickening of the outer plexiform layer (OPL); disorganization followed by loss of rod and cone outer segments; shortening of the rod and cone inner segments; retraction of bipolar cell dendrites; thinning or thickening of the inner retinal layers including inner nuclear layer, inner plexiform layer, ganglion cell layer and nerve fiber layer; opsin mislocalization; overexpression of neurofilaments; thinning of specific portions of the retina (such as the fovea or macula); loss of ERG function; loss of visual acuity and contrast sensitivity; loss of optokinetic reflexes; loss of the pupillary light reflex; and loss of visually guided behavior. In one embodiment, a method of preventing, arresting progression of or ameliorating any of the retinal changes associated with these retinal diseases is provided. In certain embodiment, the method of treatment disclosed herein improves the subject's vision or arrests and/or ameliorates vision loss.

In particular embodiments, a method of preventing, arresting progression of or ameliorating vision loss associated with an ocular disorder in the subject is provided. Vision loss associated with an ocular disorder refers to any decrease in peripheral vision, central (reading) vision, night vision, day vision, loss of color perception, loss of contrast sensitivity, or reduction in visual acuity.

In certain embodiments, the method of treatment disclosed herein increases the viability and/or homeostasis of ocular cells in the subject. In one embodiment, the ocular cell is a glial cell. In one embodiment, the ocular cell is an RPE cell. In another embodiment, the ocular cell is a photoreceptor. In another embodiment, the photoreceptor is a cone cell. In another embodiment, the ocular cell is a Muller cell. In another embodiment, the ocular cell is a bipolar cell. In yet another embodiment, the ocular cell is a horizontal cell. In another embodiment, the ocular cell is an amacrine cell. In still another embodiment, the ocular cell is a ganglion cell.

As used herein “photoreceptor function loss” means a decrease in photoreceptor function as compared to a normal, non-diseased eye or the same eye at an earlier time point. In some embodiments, the method disclosed herein may be used to increase photoreceptor function in a subject in need thereof, i.e., to improve the function of the photoreceptors or increase the number or percentage of functional photoreceptors as compared to a diseased eye (having the same ocular disease), the same eye at an earlier time point, a non-treated portion of the same eye, or the contralateral eye of the same patient. Photoreceptor function may be assessed using the functional studies described above and in the examples below, e.g., ERG, perimetry, or microperimetry, which are conventional in the art.

In some embodiments, administration of a CFH protein or pharmaceutical composition disclosed herein of the test subject results in an increase in levels of functional CFH protein. In some embodiments, ocular administration of a CFH protein or pharmaceutical composition disclosed herein to a subject results in an increase in levels of functional CFH protein in the aqueous humor such that the increased levels are 5-fold to 100-fold, 5-fold to 90-fold, 5-fold to 80-fold, 5-fold to 70-fold, 5-fold to 60-fold, 5-fold to 50-fold, 10-fold to 100-fold, 10-fold to 90-fold, 10-fold to 80-fold, 10-fold to 70-fold, 10-fold to 60-fold, or 10-fold to 50-fold of the levels functional CFH protein in the aqueous humor of a control subject or population thereof (e.g., a control patient described in the “patient selection” subsection). In some embodiments, ocular administration of a CFH protein or pharmaceutical composition disclosed herein to a subject results in an increase in levels of functional CFH protein in the vitreous humor such that the increased levels are 5-fold to 1,000-fold, 5-fold to 500-fold, 5-fold to 200-fold, 5-fold to 100-fold, 5-fold to 90-fold, 5-fold to 80-fold, 5-fold to 70-fold, 5-fold to 60-fold, 5-fold to 50-fold, 10-fold to 1,000-fold, 10-fold to 500-fold, 10-fold to 200-fold, 10-fold to 100-fold, 10-fold to 90-fold, 10-fold to 80-fold, 10-fold to 70-fold, 10-fold to 60-fold, or 10-fold to 50-fold of the levels functional CFH protein in the vitreous humor of a control subject or population thereof (e.g., a control patient described in the “patient selection” subsection).

In certain embodiments, administration of a CFH protein or pharmaceutical composition disclosed herein to the subject results in a decrease of the protein level of a biomarker positively correlated with activation of the complement pathway. For example, in certain embodiments, the administration results in a decrease of the protein level of Ba by at least 1 ng/mL, 2 ng/mL, 3 ng/mL, or 4 ng/mL 7 days post-treatment. In certain embodiments, the administration results in a decrease of the protein level of Ba by at least 1 ng/mL, 2 ng/mL, 3 ng/mL, 4 ng/mL, 5 ng/mL, 6 ng/mL, 7 ng/mL, or 8 ng/mL 28 days post-treatment. In certain embodiments, the administration results in a decrease of the protein level of Ba by at least 1 ng/mL, 2 ng/mL, 3 ng/mL, 4 ng/mL, or 5 ng/mL 56 days post-treatment. In certain embodiments, the administration results in a decrease of the protein level of C3a by at least 0.5 ng/mL, 0.6 ng/mL, 0.7 ng/mL, 0.8 ng/mL, 0.9 ng/mL, or 1 ng/mL 7 days post-treatment. In certain embodiments, the administration results in a decrease of the protein level of C3a by at least 0.5 ng/mL, 0.6 ng/mL, 0.7 ng/mL, 0.8 ng/mL, 0.9 ng/mL, or 1 ng/mL 56 days post-treatment.

### Kits

The CFH or pharmaceutical composition disclosed herein can be assembled into a pharmaceutical or diagnostic or research kit to facilitate their use in therapeutic, diagnostic or research applications. Accordingly, the present disclosure provides a kit comprising one or more containers containing a CFH or pharmaceutical composition disclosed herein and instructions for use, wherein the instructions sets a biomarker-based standard disclosed herein. In some embodiments, the kit comprises instructions for administering the CFH or pharmaceutical composition disclosed herein to a subject who has meets one of more of the biomarker tests disclosed herein and optionally has a CFH mutation disclosed herein.

Reagents useful in an assay to assess the level of a biomarker disclosed herein can also be assembled into a kit. Accordingly, in another aspect, the present disclosure provides a kit comprising one or more containers containing such reagents. For example, where the level of the biomarker is measured by an immunoassay, the kit comprises an antibody or variant thereof useful in the immunoassay. The kit can further comprise other reagents useful in such assay, such as a solid surface (e.g., microplate) and a buffer. In certain embodiments, the kit is suitable for assessing the level of a biomarker in an ocular sample. Compared to blood samples, the volume of ocular sample is typically small. Accordingly, in certain embodiments, the kit is designed to measure the level of a biomarker in a sample that has a volume smaller than or equal to 100 μL, 50 μL, 40 μL, 30 μL, 20 μL, 10 μL, 9 μL, 8 μL, 7 μL, 6 μL, 5 μL, 4 μL, 3 μL, 2 μL, or 1 μL of sample (e.g., aqueous humor sample). In certain embodiments, the kit comprises instructions for measuring the level of the protein marker, optionally further instructions to treat a patient if the level is (i) greater than or equal to a predetermined threshold if the biomarker is positively correlated with activation of the complement pathway or with inflammation or (ii) lower than or equal to a predetermined threshold if the biomarker is negatively correlated with activation of the complement pathway or with inflammation.

The instructions can define a component of instruction and/or promotion, and typically involves written instructions on or associated with packaging of the disclosure. Instructions also can include any oral or electronic instructions provided in any manner such that a user will clearly recognize that the instructions are to be associated with the kit, for example, audiovisual (e.g., videotape, DVD, etc.), internet, and/or web-based communications, etc. The written instructions may be in a form prescribed by a governmental agency regulating the manufacture, use or sale of pharmaceuticals or biological products, which instructions can also reflects approval by the agency of manufacture, use or sale for animal administration.

The kit may be designed to facilitate use of the methods described herein by researchers and can take many forms. Each of the compositions of the kit, where applicable, may be provided in liquid form (e.g., in solution), or in solid form, (e.g., a dry powder). In certain cases, some of the compositions may be reconstitutable or otherwise processable (e.g., to an active form), for example, by the addition of a suitable solvent or other species (e.g., water or a cell culture medium), which may or may not be provided with the kit.

The kits disclosed above can be combined. For example, the present disclosure also provides a kit comprising (a) one or more containers containing reagents for assessing the protein level of one or more biomarkers and (b) one or more containers containing a CFH or pharmaceutical composition disclosed herein. The kit can further comprise instructions for administering to a patient the CFH or pharmaceutical composition if the level of the biomarker is (i) greater than or equal to a predetermined threshold if the biomarker is positively correlated with activation of the complement pathway or with inflammation or (ii) lower than or equal to a predetermined threshold if the biomarker is negatively correlated with activation of the complement pathway or with inflammation.

### EXAMPLES

The disclosure is further illustrated by the following examples, which are included merely for purposes of illustration of certain aspects and embodiments of the present disclosure, and which are not intended to limit the disclosure.

### Example 1. Inhibition of the Alternative Pathway of the Complement System by Recombinant CFH in RPE Cell Culture

This example was designed to assess the impact of exogenous recombinant CFH on the activity of the alternative pathway of the complement system in a culture of RPE cells that lack sufficient complement regulation.

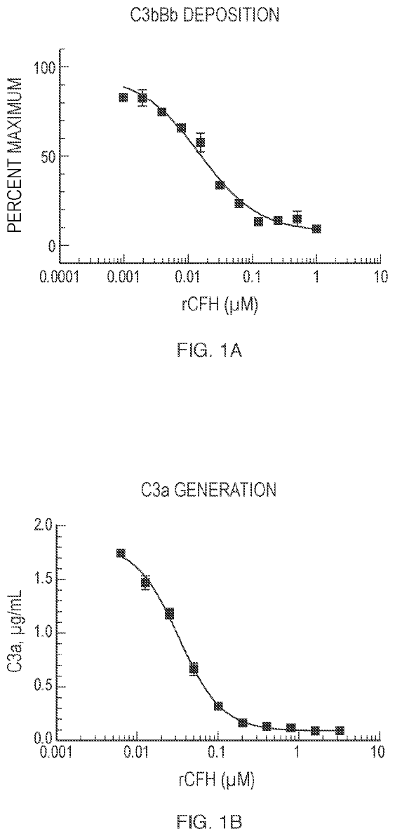

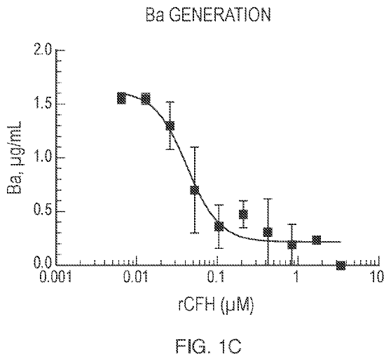

Briefly, RPE cells were subjected to a CFH level lower than the normal endogenous level by incubating RPE in CFH insufficient human serum (CFH depleted human serum with 50 ug/ml plasma-purified CFH added). These cells showed deposition of C3bBb the C3-convertase of the complement alternative pathway, as observed by immunologic detection of RPE cell-bound C3bBb by first incubating cells with a specific mouse anti-human Bb antibody followed by incubation with an horse radish peroxidase (HRP) conjugated anti-mouse antibody and finally quantitation with an HRP-sensitive colorimetric substrate. In addition, the generation of C3a and Ba, cleavage products of active C3-convertase, increased in the culture medium of these cells, as measured by commercially available ELISAs for human C3a and Ba. Recombinant, wild-type CFH protein (rCFH), having the amino acid sequence of SEQ ID NO: 1, was added in the cell culture at various concentrations. As shown in FIGS. 1A-1C, the supplementation of the rCFH reduced C3bBb deposition (FIG. 1A), generation of C3a (FIG. 1B), and generation of Ba (FIG. 1C) in a dose-dependent manner.

These results indicate that rCFH addition is able to modulate dysregulated alternative pathway of the complement system, in particular, the C3 amplification loop, on the surface of RPE cells which are of relevance to AMD pathology. It is understood that the initial levels of C3a and Ba represent the levels of these protein in the human serum added in the RPE cell culture, which were higher than the level of these proteins in an ocular sample. As a result, the doses of rCFH used in this assay also represent the levels required for reducing the levels of complement proteins in the human serum added in the RPE cell culture, which are higher than the ocular levels of these proteins.

### Example 2. Phase 1, Single-Ascending Dose Study of Recombinant CFH in Patients with Dry AMD

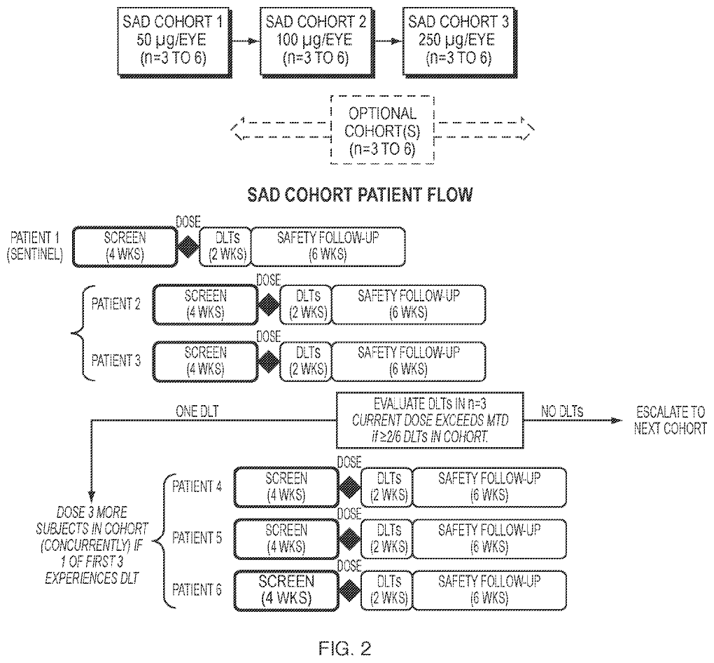

The safety, tolerability, pharmacodynamics, immunogenicity, and biomarker information of intravitreal (IVT) injection of rCFH to patients having geographic atrophy (GA) secondary to dry AMD are assessed in a Phase 1, multicenter, open-label, single-dose, dose-escalation study. In this study, rCFH is developed to restore appropriate regulation of the complement system in dry AMD patients with functionally adverse variants in the CFH gene by blocking the detrimental effects (e.g., inappropriate cell lysis, immune response) and retaining the beneficial effects (e.g., clearance of extracellular debris, repair of oxidative damage) of complement activation. The rCFH is intended to be used as a CFH replacement therapy for the treatment of dry AMD and is administered to patients by IVT administration. A diagram of a clinical study design is shown in FIG. 2.

### Objectives and End Points

The primary objectives of the study are (1) to evaluate the safety and tolerability of rCFH; and (2) to establish the single IVT maximum tolerated dose of rCFH. The endpoints for the primary objectives are: intraocular and systemic adverse events, including seriousness, severity and relationship to study drug; results of ocular examinations, clinical laboratory assessments and vital signs.

The secondary objectives are (1) to evaluate the concentration of rCFH in aqueous humor; (2) to evaluate the endogenous CFH concentration pre and post rCFH administration; and (3) to evaluate the immunogenicity of rCFH in serum and aqueous humor. The endpoints for the secondary objectives include: aqueous humor concentrations of rCFH, systemic (plasma) endogenous CFH concentrations, occurrence and severity of anti-drug antibodies in the serum and in aqueous humor.

The exploration objectives include (1) to describe the impact of rCFH on biomarkers in aqueous humor samples and (2) to describe any changes from baseline in clinical parameters such as best corrected visual acuity, retinal architecture, and pathology. The endpoints for the exploratory objectives are: aqueous humor biomarker values; plasma biomarker values, Age Related Eye Disease Study (AREDS) 9-Step Scale Score; best corrected visual acuity (BCVA) and low luminance visual acuity (LLVA)scores as assessed by Early Treatment Diabetic Retinopathy Study (ETDRS); area of geographic atrophy (GA) in mm² as assessed by color fundus photography (CFP), fundus autofluorescence (FAF), optical coherence tomography (OCT), near infrared imaging (NI), and fluorescein angiography (FA); drusen volume, total retinal and choroidal thickness, photoreceptor layer thickness, features of nascent geographic atrophy, retinal pigment epithelium (RPE) thickening and integrity of RPE layer as assessed by OCT.

### Methodology and Study Design

The rCFH is administered to 1 eye only, which is designated at Baseline (Visit 1), based on eligibility, and prior to any baseline measurements. Detailed assessments of intraocular pressure (IOP), visual acuity (VA) and function, and ophthalmic imaging is performed as described in Table 2.

TABLE 2
Schedule of Assessments for Single-Ascending Dose

| | Study Period | | | | | | | |
|---|---|---|---|---|---|---|---|---|
| | Screening | | Single Dose and Safety Follow-up | | | | | End of Study |
| | Visit ID | | | | | | | |
| | V01 | | V02 | V03 | V04 | V05 | V06 | V07 |
| | Visit Description | | | | | | | |
| | Genetic | IC/EC | Baseline | 24 hr | 1 Week | 2 Week | 4 Week | 8 Week |
| | testing | Screen | & | Post- | Safety | Safety | Safety | Safety |
| | (a) | (a) | Dosing | Dose | Visit | Visit | Visit | Visit |
| | Study Day (First Dose = Day 1) | | | | | | | |
| | Prior to | −28 to | | | 8 | 15 | 29 | 57 |
| | Dosing | −3 | 1 | 2 | (±2 days) | (±2 days) | (±2 days) | (±2 days) |
| Informed Consent (b) | X | | | | | | | |
| Oral DNA Biosampling - | X | | | | | | | |
| Genetic Testing (a) | | | | | | | | |
| Medical and Ocular History | | X | X* | | | | | |
| Inclusion/Exclusion | | X | X* | | | | | |
| Criteria (c) | | | | | | | | |
| Demographics | | X | | | | | | |
| Physical Examination | | X | X* | X | X | X | X | X |
| Vital Signs | | | | | | | | |
| Concomitant | | X | X* | | X | X | X | X |
| Medications | | | | | | | | |
| rCFH Administration | | | X | | | | | |
| Adverse Events | | | X | X | X | X | X | X |

Ocular Assessments

| | | | | | | | |
|---|---|---|---|---|---|---|---|
| Ophthalmic Biomicroscopy | OU | OU* | OU | OU | OU | OU | OU |
| Examination | | | | | | | |
| Intraocular Pressure | X | Pre/30 min | X | X | X | X | X |
| Assessment (d) | | | | | | | |
| ETDRS BCVA | OU | OU* | OU | OU | OU | OU | OU |
| ETDRS BCVA LEVA | | OU* | | | OU | OU | OU |

Imaging and Visual Function Assessments

| | | | | | | |
|---|---|---|---|---|---|---|
| Color Fundus | OU | OU | | | OU | OU |
| Photography (3-Field) | | | | | | |
| Fundus Autofluorescence | OU | OU* | | OU | OU | OU |
| Fluorescein Angiography | OU | | | | | OU |
| Near Infrared Imaging | OU | OU** | | OU | OU | OU |
| Optical Coherence Tomography | OU | OU* | OU | OU | OU | OU |
| (SD-OCT [required] and | | | | | | |
| OCT-A [optional]) | | | | | | |

Other Assessments

| | | | | | | | | |
|---|---|---|---|---|---|---|---|---|
| Identification of | | X | | | | | | |
| Study Eve (c) | | | | | | | | |
| Pregnancy Testing | | X | X* | | | | | |
| Clinical Safety | | X | X* | | X | X | X | X |
| Laboratories | | | | | | | | |
| Routine Urinalysis | | | X* | | | | | |
| Blood Sampling - | X | | | | | | | |
| Exploratory Genetics | | | | | | | | |
| Blood Sampling - ADA | | | X* | | X | X | X | X |
| Blood Sampling - | | X | Pre/2 | Post 24 | X | X | X | X |
| Endogenous CFH (e) | | | | | | | | |
| Blood Sampling - | | | X* | | X | X | X | X |
| Exploratory Biomarkers | | | | | | | | |
| Aqueous Humor Sampling (f) | | | SE | | SE | | SE | SE |

Abbreviations: ADA = anti-drug antibodies; BCVA = Best Corrected Visual Acuity; ETDRS = Early Treatment Diabetic Retinopathy Study; LLVA = Low Luminance Visual Acuity; MFD = maximum feasible dose; MTD = maximum tolerated dose; OCT = optical coherence tomography; OCT-A = OCT-angiography-; OU = oculus uterque (both eyes; eyes are tested separately); SD-OCT = spectral domain-OCT; SE = study eye.
*Assessment during a dosing visit that is obtained prior to dosing.
**Color Fundus Photography and Near Infrared Imaging are performed (prior to dosing) at the Baseline visit only if a) the investigator believes that there has been clinically significant change in vision or in ocular health, or b) the screening assessments ≥29 days prior to the baseline assessment.
(a) Saliva and blood samples for genetic testing may occur before or after assessment of eligibility criteria, but must occur prior to dosing with rCFH. Sites should allow at least 10 days for analysis.
(b) Informed consent must be obtained before any study assessment is performed.
(c) Some entry criteria apply only to the study eye. Therefore, the study eye must be identified during the screening process in order to assess entry criteria.
(d) Intraocular pressure assessments are obtained on dosing days prior to the dose and 30 minutes after the dose, with an additional sample at 60 minutes if the IOP has not resolved to ≤30 mmHg at the 30-minute assessment.
(e) Blood samples for endogenous CFH assessments are timed relative to the prior rCFH dose. Pre/2 = pre-dose and 2 hours (±5 min) post-dose. This assessment is called Biomarker Plasma B in the laboratory manual.
(f) Based on sample volume, the priorities for analysis of aqueous humor are, in order, aqueous humor biomarkers, ADA, and rCFH concentration.

A comprehensive ophthalmic examination of both the study and fellow eye (if possible) is performed by the Investigator or qualified designee at the timepoints specified in the schedule of assessments shown in Table 2. These examinations include an assessment of the visual acuity and visual function using validated assessment tools, and ocular imaging tests to visualize the appearance and pathology of the retina and associated ocular tissues. All findings are recorded in the eCRF. On dosing days, IOP is assessed pre-dose (prior to any aqueous humor sampling) and then 30 minutes after the dose. If the IOP 30 minutes post-dose exceeds 30 mmHg then the IOP is reassessed 60 minutes after the dose. If the IOP continues to exceed 30 mmHg, then standard IOP-lowering interventions (per institutional guidelines) should be followed. If IOP is ≤5 mmHg, the subject should be treated per institutional guidelines.

Only 1 eye is selected as the study eye. If both eyes meet all eye-specific entry criteria, then the eye with the poorer visual function as determined by the investigator and the subject are selected as the study eye. If neither the investigator nor the subject can make this determination, the eye with the larger GA lesion (total area) is selected as the study eye.

Safety and tolerability of a single dose of rCFH is assessed based on the occurrence of dose-limiting toxicities (DLTs). DLTs are assessed only during the DLT window, which begins at the date and time of the first dose (Day 1) and continues through the 14th calendar day after the first dose, i.e., through Study Day 15. A treated subject is considered DLT-evaluable if the subject either (a) experiences a DLT in the DLT window, or (b) experiences no DLTs during the window and continues beyond Study Day 15. If a subject has no DLTs but discontinues from the study during the DLT window, the subject is not DLT-evaluable, and an additional subject is consented and treated to achieve the required number of DLT-evaluable subjects in the same cohort.

DLTs include reduced visual acuity (VA), defined as: (1) at least 5 line (25 letter) decrease, as assessed on post-injection Day 2, 8, or 15; (2) at least a 4 line (20 letter) decrease on or before Day 8 is still present at the next scheduled visit; or (3) at least a 3 line (15 letter) decrease on or before Day 8 that persists until Day 15, intraocular inflammation defined as increase in ocular inflammation on 2 units on a standard grading scale as assessed through Day 15; elevated intraocular pressure (IOP), defined as sustained elevation of IOP characterized by an increase of 10 mmHg over the baseline value for ≥30 min; and “other DLTs” including any serious adverse event (SAE) that in the opinion of the investigator is related to the rCFH.

Cohorts of 3 to 6 DLT-evaluable subjects receive a single IVT dose of rCFH. Three escalating dose cohorts (50 μg/eye, 100 μg/eye, and 250 μg/eye) are planned (FIG. 2). Each cohort begins with dosing one sentinel subject on Day 1; the next 2 subjects in the cohort are dosed only after a favorable safety and tolerability assessment of the sentinel subject has been completed on or after Day 15 (Day 14 post dose). Subjects dosed after the sentinel subject may be dosed concurrently (at the same dose level).

If none of the first 3 DLT-evaluable subjects in a cohort experience a DLT, the dose is considered tolerable, and dose escalation proceeds to the next highest dose cohort. If exactly 1 DLT-evaluable subject experiences a DLT, 3 additional subjects are treated in order to obtain 6 DLT-evaluable subjects. The dose level for the cohort is considered tolerable if none of the 3 additional DLT-evaluable subjects experiences a DLT. If 2 of the first 3 DLT-evaluable subjects experience a DLT, the dose level is considered not tolerable.

The “3+3” single-ascending dose (SAD) study design is selected to ensure a conservative step-wise approach to investigating the MTD/MFD for rCFH. Cohorts of 3 to 6 DLT-evaluable subjects receive a single IVT dose of rCFH. Three escalating dose cohorts (50 pg/eye, 100 μg/eye, and 250 μg/eye) are planned. After review of all available data from doses ≤250 μg/eye, the sponsor can decide to open an additional cohort to evaluate a higher dose or an intermediate dose that is between a dose level found to be not tolerable and the highest previously tested tolerable dose level.

Cohorts of 3 to 6 DLT-evaluable subjects receive a single IVT dose of rCFH. Three escalating dose cohorts (50 μg/eye, 100 μg/eye, and 250 μg/eye) are planned. After review of all available data from doses ≤250 μg/eye, the sponsor may decide to open an additional cohort to evaluate a higher dose or an intermediate dose that is between a dose level found to be not tolerable and the highest previously tested tolerable dose level.

The minimum planned duration of each subject's participation is approximately 3 months: 1 month for screening, and 2 months for follow-up after the day of dosing.

A minimum of 9 subjects are planned for inclusion. Up to 18 subjects may be enrolled depending on the need for cohort expansion based on the safety response to dosing with rCFH. If a fourth cohort is opened, this number would increase by 3 to 6 more subjects.

### Eligibility Criteria

### Inclusion Criteria

Subjects must meet all of the following inclusion criteria for consideration for participation in the study: (1) at least 50 years old at the time of signed informed consent; (2) positive for the CFH 402HH variant by targeted sequencing (Table 3); (3) sufficiently clear ocular media, adequate pupillary dilation, fixation to permit quality; fundus imaging, and able to cooperate sufficiently for adequate ophthalmic visual function testing and anatomic assessment; (4) understands the full nature and purpose of the study, including possible risks of study procedures, and provides informed consent prior to initiation of any study procedure; all subjects with a reproductive potential must agree to use effective contraceptive methods for 90 days from their last dose of rCFH; (5) best corrected visual acuity (BCVA) in the study eye using Early Treatment Diabetic Retinopathy Study [ETDRS] Chart VAS of 5 to 45 letters (equivalent to Snellen VA of approximately 20/800-20/125); and (6) confirmed diagnosis of central GA in the study eye with the following characteristics:
a. GA can be multifocal, and cumulative GA lesion must reside completely within the fundus autofluorescence (FAF) imaging field (field 2, 30-degree image centered on the fovea), as confirmed by the Image Reading Center (IRC);
b. GA must be central, defined as GA that affects the foveal center point (diagnosis of GA and location relative to the foveal center point is determined by an IRC eligibility read, based on multi-modal imaging with color fundus photography [CFP], fluorescein angiography [FA], optical coherence tomography [OCT], and near infrared reflectance imaging [NIR]); and
c. Total size of all GA lesions in the study eye must be within 0.5 to 15.0 Disk Areas (DAs).

### Exclusion Criteria

A subject who meets any of the following exclusion criteria is ineligible to participate in the study:
(1) carriers of high-genetic-risk AMD variants (Table 3), to include AMD risk locus: homozygous for age-related maculopathy susceptibility protein [ARMS2]/HTRA1 p.A69S) and complete complotype (CFH 62VV+C3 102GG+complement factor B [CFB] 32RR);
(2) presence of the following ocular conditions—in the study eye:
   a. Exudative AMD or choroidal neovascularization (CNV);
   b. Any active ocular disease, disorder, or condition that that could confound the assessment of the macula or be a contraindication to IVT injection, e.g., macular hole (stage 3 or 4), GA or maculopathies due to any disease other than AMD, uveitis, uncontrolled glaucoma, ocular infection (diabetes mellitus without retinopathy is not a criterion for exclusion);
   c. Any intraocular surgery (with the exception of intraocular lens replacement surgery more than 3 months prior to consent);
   d. Aphakia or absence of the posterior capsule;
   e. History of laser therapy to the macula or fundus (exception: laser therapy to treat peripheral retinal tears is not exclusionary);
   f. Prior corneal transplant;
(3) presence of any of the following ocular conditions—in either eye:
   a. History of herpetic infection;
   b. Ongoing treatment with antiangiogenic therapies in the fellow eye or completed treatment in the study eye with antiangiogenic therapies within 5 half-lives of first rCFH dose;
   c. Concurrent disease that could require medical or surgical intervention during the study period;
   d. Active uveitis and/or vitritis (grade: trace or above);
   e. History of idiopathic or autoimmune-associated uveitis;
   f. Active infectious conjunctivitis, keratitis, scleritis, or endophthalmitis;
   g. Any ophthalmologic condition that reduces the clarity of the media and that, in the opinion of the Investigator, interferes with ophthalmologic examination;
(4) in the opinion of the Investigator, the subject has any prior or ongoing medical condition (e.g., ocular other than dry AMD, systemic, psychiatric) or clinically significant screening laboratory value that may present a safety risk, interfere with study compliance, interfere with consistent study follow-up, or confound data interpretation throughout the longitudinal follow-up period; or
(5) female subjects must not be pregnant or lactating; 6) current use of medications known to be toxic to the lens, retina, or optic nerve (deferoxamine, chloroquine/hydroxychloroquine [Plaquenil®], tamoxifen, phenothiazines, ethambutol, digoxin, and aminoglycosides). (Current use is defined as the administration of first dose of rCFH within 5 half-lives of the prohibited medication).

### Biomarkers

Blood samples for plasma isolation are obtained at the time points specified in the SOAs for analyses to identify and evaluate disease-related biomarkers, where local regulations and blood volume permits (Table 2). A specific sample for biomarker analysis is being collected in this study; however, remaining blood samples collected in this study may also be used to analyze additional biomarkers of potential clinical interest, if there is sufficient sample and where local regulations permit.

Aqueous humor samples are obtained at the time points specified in the SOAs for biomarker analyses to identify and evaluate disease-related biomarkers according to the schedule set forth in Table 2. Aqueous humor samples collected are obtained for the primary purpose of PD biomarker analysis.

TABLE 3
List of Genetic Variants Tested via Targeted Sequencing

| | Position | Reference | Alternate | | AMD Risk | Inclusion Exclusion | Eligible |
|---|---|---|---|---|---|---|---|
| Chromosome | (hg19) | Allele | Allele | dbSNPs | Nomenclature | Criteria | genotypes |
| 1 | 196,659,237 | C | T | rs1061170 | CFH: c.1204T > C | Inclusion | Positive for |
| | | | | | (p.His402=) | | CFH p.[His402=; His402=] |
| | | | | | | | (CFH 402HH) |
| 1 | 196,642,233 | G | A | rs800292 | CFH: c.184A > G | Joint | Negative for |
| | | | | | (p.Val62=) | Exclusion | CFH p.[Val62=; Val62=] AND |
| 6 | 31,914,179 | C | T | rs12614 | CFB: c.94T > C | | CFB p.[Arg32=; Arg32=] AND |
| | | | | | (p.Arg32=) | | C3 p.[Arg102Gly; Arg102Gly] |
| 6 | 31,914,180 | G | A | rs641153 | CFB: c.95A > G | | |
| | | | | | (p.Arg32=) | | |
| 19 | 6,718,387 | G | C | rs2230199 | C3: C.304C > G | | |
| | | | | | (p.Arg102Gly) | | |
| 10 | 124,215,565 | T | C | rs3750846 | ARMS2: c298- | Exclusion | Negative for |
| | | | | | 858T > C | | ARMS2: c.[298-858T > C; |
| | | | | | | | 298-858T > C] |

Abbreviations: A = adenine; AMD = age-related macular degeneration; ARMS2 = age-related maculopathy susceptibility protein 2; C = cytosine; CFB = complement factor B; CFH = complement factor H; dbSNP = single nucleotide polymorphism database; G = guanine; T = thymine

### Example 3. Results of the Phase 1, Single-Ascending Dose Study of Recombinant CFH in Patients with Dry AMID

Patients with geographic atrophy secondary to dry AMD were treated in a single ascending dose study as described in Example 2. Specifically, nine patients were equally divided in three cohorts, one for testing each rCFH dose. The sponsor reviewed all available data from doses ≤250 μg/eye, observed no dose-limiting toxicities, serious adverse events, or related adverse events, and opened an additional cohort of three patients to evaluate a higher dose of 500 pg/eye. As a result, in this clinical study, the patients were treated at four different doses, namely, 50 μg, 100 μg, 250 μg, and 500 μg per eye. No dose-limiting toxicities, serious adverse events, or related adverse events were observed during the course of treatment.

It is understood that measurement of protein biomarkers in ocular samples has been technically challenging, given the limited sample volumes and the low levels of many protein biomarkers in ocular samples. In a clinical setting, where a plurality of biomarkers are assessed from the same sample, it would be desirable to conduct multiplex measurements of the multiple protein biomarkers under a single level of dilution. The inventors successfully measured the levels of IL-6, IL-8, VEGF-A, Eotaxin-2, and CCL2 from undiluted and 1:4 diluted aqueous humor samples from patients having wet AMD or cataract using the ECLIA method of Meso Scale Discovery (MSD). The inventors also successfully measured the levels of CXCL5, IL-10, and IL-18 from undiluted aqueous humor samples from the same patients using the same method. Additional biomarkers (up to ten in total) can be measured in a U-PLEX multiplex assay.

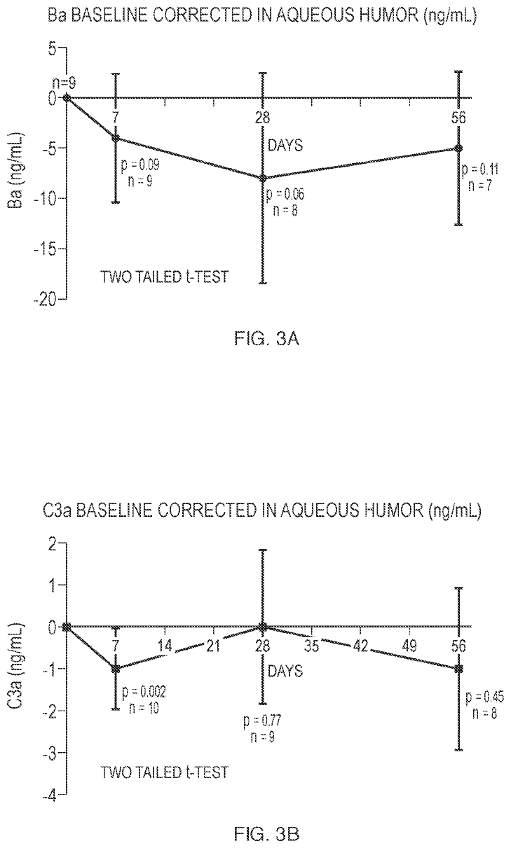

In aqueous humor, supraphysiological levels of CFH were achieved and maintained, along with a reduction in complement activation biomarkers. The baseline levels of Ba in the treated patients ranged from 8.4 ng/mL to 41.2 ng/mL, whereas the Ba levels in healthy patients generally ranged from 6.2 ng/mL to 11.5 ng/mL and averaged 7.8 ng/mL. The baseline levels of C3a in the treated patients ranged from 1.5 ng/mL to 9.6 ng/mL, whereas the C3a levels in healthy patients generally ranged from 1.6 ng/mL to 2.9 ng/mL and averaged 2.2 ng/mL. Given that all the patients appeared to have received sufficient amounts of rCFH to induce protein level changes of certain biomarkers at least within 7 days after administration, the results of all the patients receiving different doses were pooled. As shown in FIGS. 3A-3B, the levels of complement activation fragments Ba and C3a were generally higher in the patients at pre-treatment stage, reflecting the state of uncontrolled ocular C3-convertase activity. After treatment, the levels of activation fragments were reduced, indicating the ability of rCFH to regulate excessive C3-convertase activity. This is the first demonstration in a clinical study that in patients with geographic atrophy, intravitreal administration of a complement regulator can restore complement regulation and reduce levels of pathogenically relevant complement products.

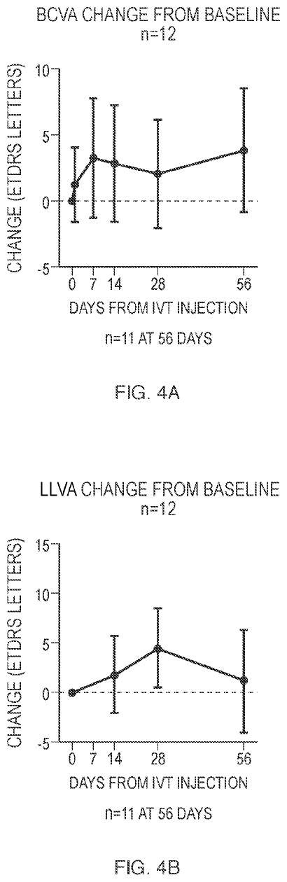

The BCVA and LLVA scores of the patients were also measured. As shown in FIGS. 4A-4B, the rCFH treatment stabilized the disease progression. There also appeared to be a trend of improvement of visual outcome, but the difference did not arise to a level of statistical significance. More significant improvement of clinical outcome is expected be observed in a phase 2 clinical trial with more patients enrolled and multiple doses of rCFH administered.

### Example 4—Phase 2, Multiple Dose Study of Recombinant CFH (rCFH) in Patients with Dry AMD

The safety, tolerability, pharmacodynamics, and immunogenicity of repeat intravitreal (IVT) injections of rCFH to patients having geographic atrophy (GA) secondary to dry AMD were assessed in a Phase 2, multicenter, open-label, multiple dose study. An optimal dose of rCFH was developed and safety, tolerability, immunogenicity, PK (measured as concentration of CFH in aqueous humor)/PD, and early clinical effect were assessed. This phase 2 clinical study was designed, in part, on the basis of the phase 1 Single Ascending Dose Study described in Example 3.

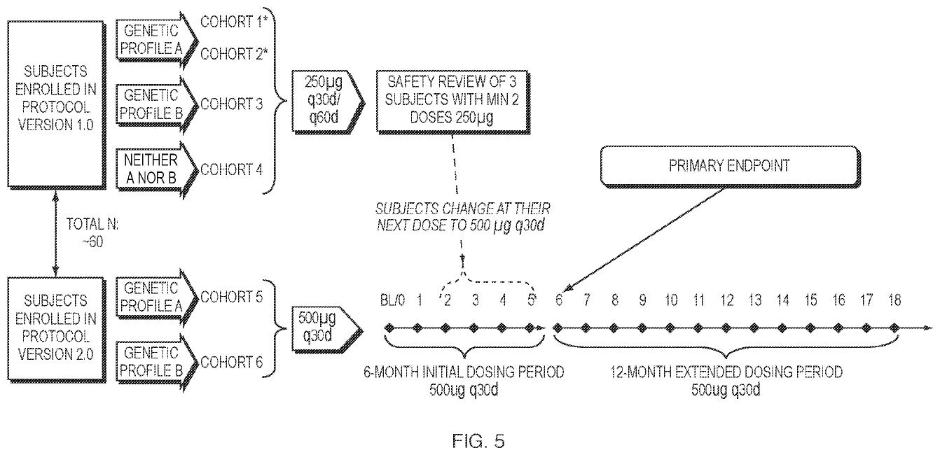

Repeat doses of rCFH were administered by IVT injection to subjects with dry AMD. The initial MD study dose (dose level) of up to 250 μg was based on safety data from single- and repeat-dose (3 monthly doses) IVT toxicology studies in cynomolgus monkeys, PK modeling and simulation of the profiles of rCFH after a single IVT injection in cynomolgus monkeys, and the endogenous level of CFH in human RPE (the target ocular tissue of rCFH replacement therapy in dry AMD patients), as well as data from initial dose cohorts in the Phase 1 SAD Study as described in Example 3. The initial 250 μg dose level was based on the clinical experience with 50 μg, 100 μg, and 250 μg in the Phase 1 SAD Study (as described in Example 2). The inclusion of the 500 μg dose level was supported by nonclinical toxicology studies and the clinical experience with 50 μg, 100 μg, 250 μg, and 500 μg in the Phase 1 SAD Study. A diagram of a clinical study design for Phase 2 is shown in FIG. 5.

### Objectives and End Points

The primary objectives of the study were to establish the safety and tolerability of multiple IVT injections of rCFH. The endpoints for the primary objectives were: intraocular and systemic adverse events, including seriousness, severity and relationship to study drug; results of ocular examinations; and clinical laboratory assessments.

The secondary objectives of the study were: (1) to describe the clinical effect of rCFH IVT injection; (2) to evaluate the total CFH levels in aqueous humor after rCFH IVT injection; and (3) to evaluate the immunogenicity of rCFH in serum after rCFH IVT injection. The endpoints for the secondary objectives include: change from baseline in best corrected visual acuity (BCVA) scores as assessed by Early Treatment Diabetic Retinopathy Study (ETDRS); change from baseline in area and/or rate of progression of GA as assessed by fundus autofluorescence (FAF) and optical coherence tomography (OCT); change from baseline in low luminance visual acuity (LLVA), and derived low luminance deficit (LLD) value (BCVA−LLVA); total CFH concentration levels in aqueous humor; and generation of anti-drug antibodies in the serum.

The exploration objectives of the study included (1) to describe any changes from baseline in retinal architecture and pathology after rCFH IVT injection; (2) to describe any changes from baseline in functional parameters (reading speed) after rCFH IVT injection; (3) to describe any changes from baseline in quality of life after rCFH IVT injection; (4) to describe the impact of rCFH IVT injection on biomarkers in aqueous humor sample. The endpoints for the exploratory objectives include: (1) descriptive analyses of changes from baseline in: retinal fluid/hemorrhage or development of choroidal neovascularization (CNV) by color fundus photography (CFP), fluorescein angiography (FA), and OCT; drusen morphology and volume, total retinal and photoreceptor layer thickness, features of nascent GA and rate of progression, RPE thickening, and integrity of RPE layer as assessed by FAF, OCT, optical coherence tomography-angiography (OCT-A) (optional), and near infrared reflectance imaging (NIR); (2) change from baseline in MNRead (Minnesota Low-vision Reading Test) scores; (3) change from baseline in NEI-VFQ-25 (National Eye Institute Visual Functioning Questionnaire-25) scores; and (4) change from baseline in aqueous humor complement proteins, complement split products, and related protein values as potential biomarkers of (pharmacodynamics) PD.

### Methodology

Subjects were assigned to 1 of 6 cohorts, based on subject genetic profile and location of GA (discussed below in “Inclusion Criteria” section).
Multiple dose (MD) cohort 1 subjects had genetic profile A and noncentral GA;
MD cohort 2 subjects had genetic profile A and central GA;
MD Cohort 3 subjects had genetic profile B and either central or noncentral GA;
MD cohort 4 included subjects who participated in the Phase 1 SAD Study (described in Example 2) and completed their EOS Visit, who had neither genetic profile A (CFH 402HH) nor B (Rare CFH/complotype), but who otherwise met all eligibility criteria and wish to continue receiving rCFH. Treatment naïve subjects that did not participate in the Phase 1 SAD Study and who meet all eligibility criteria (except the genetic profiles criteria) may also be included in MD cohort 4. Patients were enrolled in MD cohort 4 at the discretion of the medical monitor, in consultation with the Investigator;
MD cohort 5 subjects had genetic profile A; and
MD cohort 6 subjects had genetic profile B.

Subjects in MD cohorts 1-4 (as described in this section) received 250 μg rCFH either monthly or every other month (EOM), based on their initial treatment assignment. For subjects in MD cohorts 1-4, dose escalation from 250 μg to 500 μg monthly was considered when at least 3 subjects had completed at least 2 doses of rCFH at 250 μg and the available safety data had been assessed by the MRC. Subjects in MD cohorts 5 and 6 received 500 μg rCFH monthly. The study had two periods of follow-up: a 6-month initial dosing period and a 12-month extended dosing period. Based on MRC reviews of ongoing data, the dose and/or dosing frequency could be adjusted during the 12-month extended dosing period. An overview of MD cohorts is provided in Table 4.

TABLE 4
Overview of MD Cohorts

| | GA | | | Dose Level |
|---|---|---|---|---|
| MD Cohort | Location | Genetics | N | (μg) |
| MD Cohort 1 | Noncentral | Profile A | ~60 Total | 250 μg Monthly/EOM- |
| | | (CFH 402HH, no ARMS2/HTRA1) | | 500 μg Monthly |
| MD Cohort 2 | Central | Profile A | | 250 μg Monthly/EOM- |
| | | (CFH 402HH, no ARMS2/HTRA1) | | 500 μg Monthly |
| MD Cohort 3 | Noncentral/ | Profile B | | 250 μg Monthly- |
| | central | (Rare CFH/complete complotype) | | 500 μg Monthly |
| MD Cohort 4 | Noncentral/ | Neither Profile A nor Profile B | | 250 μg Monthly- |
| | central | | | 500 μg Monthly |
| MD Cohort 5 | Noncentral/ | Profile A | | 500 μg Monthly |
| | central | (CFH 402HH) | | |
| MD Cohort 6 | Noncentral/ | Profile B | | 500 μg Monthly |
| | central | (Rare CFH/complete complotype) | | |

Abbreviations: CFH = complement factor H; EOM = every other month; GA = geographic atrophy; ARMS2/HTRA1 = age-related maculopathy susceptibility 2/high temperature requirement A serine peptidase 1; MD = multiple dose; N = number of subjects

The rCFH was administered to 1 eye only, which was designated during the screening process based on eligibility and prior to any baseline measurements. If both eyes met all eye-specific entry criteria, then the study eye was determined by the Investigator and subject together. If neither the Investigator nor the subject could make this determination, the eye with the larger GA lesion (total area) would be selected as the study eye. The fellow eye may be followed for comparative analyses, where appropriate. A schedule of assessments for 6-month initial dosing period and 12-month extended dosing period are described in Table 5 and Table 6, respectively.

TABLE 5
Schedule of Assessments -6-Month Initial Dosing Period (Primary Endpoint)

| | Visit Description | | | | | | | | | | | |
|---|---|---|---|---|---|---|---|---|---|---|---|---|
| | Screening | | | 48 hr | | Week | | | | | | |
| | Genetic | IC/EC | | Post-dose | Week | 2 | Month | Month | Month | Month | Month | Month |
| | Testing | Screen | Baseline | Phone | 1 | Phone | 1 | 2 | 3 | 4 | 5 | 6 |
| | ^{a} | ^{a} | ^{a} | Call | Visit | Call | Visit | Visit | Visit | Visit | Visit | Visit |
| | Study Day (First Dose = Day 1) | | | | | | | | | | | |
| | Prior | −35 to | | 3 | 8 | 15 | 31 | 61 | 91 | 121 | 151 | 181 |
| | to Day 1 | −3 | 1 | (±1) | (±2) | (±1) | (±4) | (±4) | (±4) | (±4) | (±4) | (±4) |
| Informed Consent | x | | | | | | | | | | | |
| for Genetic Testing | | | | | | | | | | | | |
| Informed Consent for 6- | | x | | | | | | | | | | |
| Month Initial Dosing | | | | | | | | | | | | |
| Period ^{b} | | | | | | | | | | | | |
| Informed Consent for | | | | | | | | | | | | x |
| 12-Month Extended | | | | | | | | | | | | |
| Dosing Period | | | | | | | | | | | | |
| Oral DNA Biosample - | x | | | | | | | | | | | |
| Genetic Testing * | | | | | | | | | | | | |
| Historical imaging | | | Obtained between Baseline and Month 3 | | | | | | | | | |
| (including imaging | | | | | | | | | | | | |
| type) and visual acuity | | | | | | | | | | | | |
| Medical and Ocular | | x | x | | | | | | | | | |
| History *^{c} | | | | | | | | | | | | |
| Inclusion/Exclusion | | x | x | | | | | | | | | |
| Criteria *^{d} | | | | | | | | | | | | |
| Demographics | | x | | | | | | | | | | |
| Complete Physical | | x | | | | | | | | | | x |
| Examination | | | | | | | | | | | | |
| Symptom-Directed | | | x | | | | x | x | x | x | x | |
| Physical | | | | | | | | | | | | |
| Examination * | | | | | | | | | | | | |
| ECG | | x | | | | | | | | | | |
| Vital Signs *^{e} | | x | x | | | | x** | x | x** | x | x** | x** |
| Concomitant | | x | x | x | x | x | x | x | x | x | x | x |
| Medications * | | | | | | | | | | | | |
| rCFH Administration | | | SE | | | | SE | SE | SE | SE | SE | SE ^{f} |
| (monthly) | | | | | | | | | | | | |
| Adverse Events | | | x | x | x | x | x | x | x | x | x | x |

Ocular Assessments

| | | | | | | | | | |
|---|---|---|---|---|---|---|---|---|---|
| Complete Ophthalmic | OU | OU | OU | OU | OU | OU | OU | OU | OU |
| Examination * | | | | | | | | | |
| Intraocular Pressure | OU | OU ^{g} | OU | OU ^{g} | OU ^{g} | OU ^{g} | OU ^{g} | OU ^{g} | OU ^{g} |
| Assessment ^{g} | | | | | | | | | |
| ETDRS BCVA * | OU | OU | OU | OU | OU | OU | OU | OU | OU |
| ETDRS BCVA LLVA * | OU | | | | | OU | | | OU |

Imaging and Visual Function Assessments
(Imaging assessments may be optionally performed at any in-
clinic visit if deemed medically necessary for subject safety.)

| | | | | |
|---|---|---|---|---|
| Color Fundus | OU | OU ^{h} | OU | OU |
| Photography (7-Field) * | | | | |
| Fundus | OU | OU ^{h} | OU | OU |
| Autofluorescence * | | | | |
| Fluorescein | OU | OU ⁱ | | |
| Angiography * | | | | |
| Near Infrared | OU | OU ^{h} | OU | OU |
| Imaging * | | | | |
| Optical Coherence | OU | OU ^{h} | OU | OU |
| Tomography (SD-OCT | | | | |
| [required] and | | | | |
| OCT-A [optional]) * | | | | |
| NEI-VFQ-25 * | | x | | OU |
| MNRead * | | OU | | OU |

Other Assessments

| | | | | | | | | | |
|---|---|---|---|---|---|---|---|---|---|
| Identification of | | x | | | | | | | |
| Study Eye ^{d} | | | | | | | | | |
| Pregnancy | | x | | | | | | | x^{f} |
| Testing ^{j} | | | | | | | | | |
| Clinical Safety | | x | x | | x | | x | | x |
| Laboratory | | | | | | | | | |
| Assessments *^{k} | | | | | | | | | |
| Routine | | x | | | | | | | x |
| Urinalysis ^{l} | | | | | | | | | |
| Blood Sampling - | x | | | | | | | | x |
| Exploratory | | | | | | | | | |
| Genetics ^{a} | | | | | | | | | |
| Blood Sampling | | | x | x | x | | x | | x |
| for serum ADA * | | | | | | | | | |
| Aqueous Humor | | | SE | SE** | SE | SE** | SE | SE** | SE** |
| Sampling * | | | | | | | | | |

Abbreviations: ADA = anti-drug antibodies; BCVA = best corrected visual acuity; EOM = every other month; ECG = electrocardiogram; ETDRS = Early TreatmentDiabetic Retinopathy Study; IOP = intraocular pressure; LLVA = low luminance visual acuity; MNRead = Minnesota Low-vision Reading Test; NEI-VFQ-25 = National Eye Institute Visual Functioning Questionnaire-25; OCT = optical coherence tomography; OCT-A = OCT-angiography; OU = oculus uterque (both eyes; eyes will be tested separately); SD-OCT = spectral domain-OCT; SE = study eye
* Assessment obtained prior to dosing, as applicable.
**Assessment obtained on dosing visits only, prior to dosing and at month 6 if the subject does not agree to continue into the 12-Month Extended Dosing Period.
^{a} Results of saliva or blood samples for genetic testing must be available before dosing. Prior genetic testing results from a Sponsor-qualified assay may be acceptable after consultation with the medical monitor. The Baseline visit may be performed over several days if necessary but must be completed within 1 week.
^{b} Informed consent must be obtained before any study assessment is performed.
^{c} Medical and ocular history also includes social history and family ophthalmic history.
^{d} Some entry criteria apply only to the SE. Therefore, the SE must be identified during the screening process in order to assess entry criteria.
^{e} Vital sign measurements include systolic and diastolic blood pressures, heart rate, respiratory rate, and body temperature. When procedures overlap and are scheduled to occur at the same time point, vital sign measurements should precede any blood collection.
^{f} Dose administration and pregnancy testing at this visit only occurs if the subject has consented to the 12-Month Extended Dosing Period.
^{g} On dosing days, IOP assessments are obtained prior to the dose and 30 ± 15 minutes after the dose, with an additional sample at 60 ± 15 minutes if the IOP has not resolved to ≤30 mmHg at the 30-minute assessment. If the IOP continues to exceed 30 mmHg, then standard IOP-lowering interventions (per institutional guidelines) should be followed. If IOP is ≤5 mmHg, the subject should be treated per institutional guidelines. Regardless of whether applanation tonometry or a tonopen is used to measure IOP, the same method should be used for the subject for the entirety of their participation in this study.
^{h} These baseline imaging assessments are only required if the screening images were obtained >35 days prior to baseline. They may be redone at baseline if the Investigator deems it clinically relevant, or if there were quality issues with the screening images.
ⁱ Baseline assessment of fluorescein angiography is not required, but may be assessed if the Investigator deems it clinically relevant, or if there were quality issues with the screening images.
^{j} Highly sensitive (serum or urine) human chorionic gonadotropin pregnancy test as needed for women of childbearing potential.
^{k} Clinical safety laboratory assessments include the following: hematology (platelet count, red blood cell count, hemoglobin count, hematocrit, mean corpuscular volume, mean corpuscular hemoglobin, percent reticulocytes, and white blood cell count with differential) and clinical chemistry (blood urea, nitrogen, creatinine, glucose, potassium, sodium, total calcium, aspartate aminotransferase, alanine aminotransferase, alkaline phosphatase, total and directbilirubin, total protein, standard electrolytes - magnesium, phosphate, and chloride). Baseline clinical safety labs do not need to be drawn if screening labs were drawn within 7 days prior to baseline.
^{l} Routine urinalysis includes macroscopic and microscopic examination.

TABLE 6
Schedule of Assessments - 12-Month Extended Dosing Period

| | Visit Description | | | | | | | | | | | |
|---|---|---|---|---|---|---|---|---|---|---|---|---|
| | Month | Month | Month | Month | Month | Month | Month | Month | Month | Month | Month | |
| | 7 | 8 | 9 | 10 | 11 | 12 | 13 | 14 | 15 | 16 | 17 | EOS |
| | Visit | Visit | Visit | Visit | Visit | Visit | Visit | Visit | Visit | Visit | Visit | Visit |
| | Study Day (First Dose = Day 1) | | | | | | | | | | | |
| | 210 | 241 | 271 | 301 | 331 | 361 | 391 | 421 | 451 | 481 | 501 | 531 |
| | (±4) | (±4) | (±4) | (±4) | (±4) | (±4) | (±4) | (±4) | (±4) | (±4) | (±4) | (±4) |
| Complete Physical | | | | | | x | | | | | | x |
| Examination | | | | | | | | | | | | |
| Symptom-Directed | x | x | x | x | x | | x | x | x | x | x | |
| Physical Examination | | | | | | | | | | | | |
| Vital Signs * ^{a} | x | x | x | x | x | x | x | x | x | x | x | x |
| Concomitant Medications | x | x | x | x | x | x | x | x | x | x | x | x |
| rCFH Administration | SE | SE | SE | SE | SE | SE | SE | SE | SE | SE | SE | |
| Adverse Events | x | x | x | x | x | x | x | x | x | x | x | x |

Ocular Assessments

| | | | | | | | | | | | | |
|---|---|---|---|---|---|---|---|---|---|---|---|---|
| Complete Ophthalmic | OU | OU | OU | OU | OU | OU | OU | OU | OU | OU | OU | OU |
| Examination * | | | | | | | | | | | | |
| Intraocular | OU | OU | OU | OU | OU | OU | OU | OU | OU | OU | OU | OU |
| Pressure Assessment | | | | | | | | | | | | |
| ETDRS BCVA * | OU | OU | OU | OU | OU | OU | OU | OU | OU | OU | OU | OU |
| ETDRS BCVA LLVA * | | | OU | | | OU | | | OU | | | OU |

Imaging and Visual Function Assessments
(Imaging assessments may be optionally performed at any in-
clinic visit if deemed medically necessary for subject safety)

| | | | | |
|---|---|---|---|---|
| Color Fundus | OU | OU | OU | OU |
| Photography (7-Field) * | | | | |
| Fundus Autofluorescence * | OU | OU | OU | OU |
| Fluorescein Angiography * | | OU | | OU |
| Near Infrared Imaging * | OU | OU | OU | OU |
| Optical Coherence | OU | OU | OU | OU |
| Tomography (SD-OCT | | | | |
| [required] and | | | | |
| OCT-A [optional]) * | | | | |
| NEI-VFQ-25 * | | OU | | OU |
| MNRead * | | OU | | OU |

Other Assessments

| | | | | |
|---|---|---|---|---|
| Pregnancy | | | | x |
| Testing ^{c} | | | | |
| Clinical Safety | x | x | x | x |
| Laboratory | | | | |
| Assessments *^{d} | | | | |
| Routine | x | x | x | x |
| Urinalysis ^{e} | | | | |
| Blood Sampling | | x | | x |
| for serum ADA | | | | |
| Aqueous Humor | SE | SE | SE | SE |
| Sampling * | | | | |

Abbreviations: ADA = anti-drug antibodies; BCVA = best corrected visual acuity; EOS = End of Study; ECG = electrocardiogram; ETDRS = Early Treatment Diabetic Retinopathy Study; hr = hour; IOP = intraocular pressure; LLVA = low luminance visual acuity; MNRead = Minnesota Low-vision Reading Test; NEI-VFQ-25 = National Eye Institute Visual Functioning Questionnaire-25; OCT = optical coherence tomography; OCT-A = OCT-angiography; OU = oculus uterque (both eyes; eyes will be tested separately); SD-OCT=spectral domain-OCT; SE=study eye.
* Assessment obtained prior to dosing, as applicable.
^{a} Vital sign measurements include systolic and diastolic blood pressures, heart rate, respiratory rate, and body temperature. When procedures overlap and are scheduled to occur at the same time point, vital sign measurements should precede any blood collection.
^{b} IOP assessments are obtained prior to the dose and 30 ± 15 minutes after the dose, with an additional sample at 60 ± 15 minutes if the IOP has not resolved to ≤30 mmHg at the 30-minute assessment. If the IOP continues to exceed 30 mmHg, then standard IOP-lowering interventions (per institutional guidelines) should be followed. If IOP is ≤5 mmHg, the subject should be treated per institutional guidelines. Regardless of whether applanation tonometry or a tonopen is used to measure IOP, the same method should be used for the subject for the entirety of their participation in this study.
^{c} Highly sensitive (serum or urine) human chorionic gonadotropin pregnancy test as needed for women of childbearing potential.
^{d} Clinical safety laboratory assessments include the following: hematology (platelet count, red blood cell count, hemoglobin count, hematocrit, mean corpuscular volume, mean corpuscular hemoglobin, percent reticulocytes, and white blood cell count with differential) and clinical chemistry (blood urea nitrogen, creatinine, glucose, potassium, sodium, total calcium, aspartate aminotransferase, alanine aminotransferase, alkaline phosphatase, total and directbilirubin, total protein, standard electrolytes - magnesium, phosphate, and chloride).
^{e} Routine urinalysis includes macroscopic and microscopic examination.

### Genetic Profiles

Genetic variants tested via targeted sequencing are listed in Table 7.

TABLE 7
List of Genetic Variants Tested via Targeted Sequencing

| | Position | Reference | Alternate | | AMD Risk | Genetic | Genetic |
|---|---|---|---|---|---|---|---|
| Chromosome | (hg19) | Allele | Allele | dbSNPs | Nomenclature | Profile A | Profile B |
| 1 | 196,659,237 | C | T | rs1061170 | CFH: c.1204T > C | Positive for CFH | Any genotype |
| | | | | | (p.His402=) | p.[His402=; | |
| | | | | | | His402=] | |
| | | | | | | (CFH 402HH) | |
| 10 | 124,215,565 | T | C | rs3750846 | ARMS2: c.298-858T > C | Any genotype | Any genotype |
| 1 | 196,642,233 | G | A | rs800292 | CFH: c.184A > G | Negative for complete | Positive for complete |
| | | | | | (p.Val62=) | complement | complement |
| 6 | 31,914,179 | C | T | rs 12614 | CFB: c.94T > C | (CFH p.[Val62=; | (CFH p.[Val62=; |
| | | | | | (p.Arg32=) | Val62=] | Val62=] |
| 6 | 31,914,180 | G | A | rs641153 | CFB: c.95A > G | AND | ANDCFB |
| | | | | | (p.Arg32=) | CFB p.[Arg32=; | p.[Arg32=; |
| 19 | 6,718,387 | G | C | rs2230199 | C3: c.304C > G | Arg32=] | Arg32=] |
| | | | | | (p.Arg102Gly) | AND | AND |
| | | | | | | C3 p.[Arg102Gly; | C3 p.[Arg102Gly; |
| | | | | | | Arg102Gly]) | Arg102Gly]) |
| 1 | 196,621,252 | G | C | rs142266551 | CFH: c.5G > C | Negative | OR |
| | | | | | (p.Arg2Thr) | for all | Positive for any |
| 1 | 196,621,254 | C | G | rs139254423 | CFH: c.7C > G | | one of the 20 rare |
| | | | | | (p.Leu3 Val) | | missense CFH |
| 1 | 196,642,206 | C | T | rs757785149 | CFH: c.157C > T | | variants |
| | | | | | (p.Arg53Cys) | | |
| 1 | 196,642,207 | G | A | rs757785149 | CFH: c.158G > A | | |
| | | | | | (p.Arg53His) | | |
| 1 | 196,642,221 | T | G | rs141336681 | CFH: c.172T > G | | |
| | | | | | (p.Ser58Ala) | | |
| 1 | 196,643,011 | A | G | rs1239695899 | CFH: c.269A > G | | |
| | | | | | (p.Asp90Gly) | | |
| 1 | 196,645,156 | G | A | rs147002633 | CFH: c.388G > A | | |
| | | | | | (p.Asp130Asn) | | |
| 1 | 196,646,702 | G | A | rs1300493671 | CFH: c.524G > A | | |
| | | | | | (p.Arg175Gln) | | |
| 1 | 196,646,702 | G | C | rs139360826 | CFH: c.524G > C | | |
| | | | | | (p.Arg175Pro) | | |
| 1 | 196,648,794 | A | G | rs774239374 | CFH: c.661A > G | | |
| | | | | | (p.Ile221 Val) | | |
| 1 | 196,654,310 | C | T | rs142937931 | CFH: c.907C > T | | |
| | | | | | (p.Arg303Trp) | | |
| 1 | 196,654,311 | G | A | rs766408580 | CFH: c.908G > A | 20 rare | |
| | | | | | (p.Arg303Gln) | missense | |
| 1 | 196,659,231 | C | A | rs201671665 | CFH: c.1198C > A | CFH | |
| | | | | | (p.Gln400Lys) | variants | |
| 1 | 196,683,035 | C | G | rs570523689 | CFH: c.1507C > G | | |
| | | | | | (p.Pro503Ala) | | |
| 1 | 196,694,253 | A | G | rs757756991 | CFH: c.1699A > G | | |
| | | | | | (p.Arg567Gly) | | |
| 1 | 196,695,675 | G | T | rs143237092 | CFH: c.1949G > T | | |
| | | | | | (p.Gly650Val) | | |
| 1 | 196,706,677 | G | T | rs515299 | CFH: c.2669G > T | | |
| | | | | | (p.Ser890Ile) | | |
| 1 | 196,709,833 | C | T | rs145975787 | CFH: c.2867C > T | | |
| | | | | | (p.Thr956Met) | | |
| 1 | 196,716,328 | G | A | rs761877050 | CFH: c.3581G > A | | |
| | | | | | (p.Gly1194Asp) | | |
| 1 | 196,716,375 | C | T | rs121913059 | CFH: c.3628C > T | | |
| | | | | | (p.Arg1210Cys) | | |

Abbreviations: A = adenine; AMD = age-related macular degeneration; ARMS2 = age-related maculopathy susceptibility 2; C = cytosine; C3 = complement component 3; CFB = complement factor B; CFH = complement factor H; dbSNP = single nucleotide polymorphism database; G = guanine; T = thymine

Emerging data, including safety, PK/PD, biomarker, and clinical data, were reviewed on an ongoing basis, and may be reviewed at specific timepoints during the study (e.g., when all ongoing subjects have been treated with rCFH for 12 weeks and again at 6 and 12 months during the treatment period).

Data was collected on demographics, medical and ocular history, family history, and concomitant medications. Additionally, ophthalmic anatomic assessments and multi-modal imaging, genetic and biomarker analyses, and aqueous humor sampling was done for each enrolled subject.

For evaluation of post treatment effect, historical data, including visual acuity and anatomical endpoints such as rate of progression of GA (measured from diagnosis to study screen where available), drusen volume, and retinal architecture, can also be used.

### Study Duration

The planned duration of each subject's participation in the initial dosing period was approximately 7 months: 1 month for screening and 6 months for dosing and follow-up. The planned duration of each subject's participation in the extended dosing period was approximately 12 months of dosing and follow-up.

Approximately 60 subjects are in the study.

### Eligibility Criteria

### Inclusion Criteria

Subjects must meet all of the following inclusion criteria for consideration for participation in the study. (1) at least 50 years old at the time of signed informed consent; subjects younger than 50 may be considered for participation after consultation; (2) subjects in MD cohorts 5 and 6 must have one of the following genetic profiles (see Table 7):
a. Genetic profile A (CFH 402HH)—positive for the CFH 402HH variant and negative for rare missense CFH variants and complete complotype;
b. Genetic profile B (Rare CFH/complete complotype)—positive for at least one of the rare high-risk missense CFH genetic variants or complete complotype (CFH 62VV+C3 102GG+CFB 32RR);
   (3) BCVA in the study eye of 24 to 83 letters using the ETDRS Chart VA Scale (approximately equivalent to Snellen VA of 20/25 to 20/320); (4) confirmed diagnosis of central GA in the study eye with the following characteristics:
a. total GA area must be ≥1.25 and ≤17.5 mm² (0.5- and 7-disc areas (DA), respectively), determined by screening images of fundus autofluorescence (FAF);
b. if GA is multifocal, at least one focal lesion must be: 1.25 mm² (0.5 DA); and
   (5) the entire atrophic lesion must be able to be photographed within field 2 on FAF; (6) sufficiently clear ocular media, adequate pupillary dilation, fixation to permit quality fundus imaging, and able to cooperate sufficiently for adequate ophthalmic visual function testing and anatomic assessment in the study eye; (7) understands the full nature and purpose of the study, including possible risks of study procedures, and provides informed consent prior to initiation of any study procedure; all subjects with a reproductive potential must agree to use effective contraceptive methods through the end of study (EOS) Visit.

### Exclusion Criteria

A subject who meets any of the following exclusion criteria was ineligible to participate in the study:
(1) presence of the following ocular conditions—in the study eye:
   a. Any history of exudative AMD or choroidal neovascularization (CNV);
   b. Any active ocular disease or condition that could confound the assessment of the macula or be a contraindication to IVT injection, e.g., macular hole (any stage, including lamellar hole), epiretinal membrane, GA or maculopathies due to any disease other than AMD, uncontrolled glaucoma (intraocular pressure (IOP) of >24 mmHg when on 2 or more IOP-lowering medications), severe glaucoma (cup to disc ratio of 0.8 or greater), ocular infection of any nature, diabetic retinopathy graded as moderate non-proliferative diabetic retinopathy or worse, with or without diabetic macular edema);
   c. Any intraocular surgery, with the exception of stable intraocular lens replacement surgery more than 3 months prior to consent (e.g., any glaucoma surgeries, including microinvasive glaucoma surgery, and/or any intraocular surgery requiring vitrectomy);
   d. Aphakia or complete absence of the posterior capsule;
   e. History of laser therapy to the macula or fundus or extensive laser (e.g., pan-retinal photocoagulation) to the retina (exception: laser therapy to treat peripheral retinal tears is not exclusionary); and
   f. Prior corneal transplant;
(2) presence of any of the following ocular conditions—in either eye:
   a. History of herpetic infection;
   b. Concurrent disease that could require medical or surgical intervention during the study period;
   c. Active uveitis and/or vitritis (grade: trace or above);
   d. History of idiopathic or autoimmune-associated uveitis;
   e. Active blepharitis, conjunctivitis, keratitis, episcleritis, scleritis, or endophthalmitis; and
   f. Any ophthalmologic condition that reduces the clarity of the media and that, in the opinion of the Investigator, interferes with ophthalmologic examination;
(3) in the opinion of the Investigator, the subject had any prior or ongoing medical condition (e.g., ocular other than dry AMD, systemic, psychiatric) or clinically significant screening laboratory value that may present a safety risk, interfere with study compliance, interfere with consistent study follow-up, or confound data interpretation throughout the longitudinal follow-up period; (4) female subjects must not be pregnant or lactating, nor plan to become pregnant during the study; (5) current use of medications known to be toxic to the lens, retina, or optic nerve (deferoxamine, chloroquine/hydroxychloroquine [Plaquenil®], tamoxifen, phenothiazines, ethambutol, digoxin, and aminoglycosides). (Current use is defined as the administration of first dose of rCFH within 5 half-lives of the prohibited medication). Past use of these medications, without history of ocular side effects, is not considered exclusionary; or (6) use of any investigational new drug or other experimental treatment in the last 6 months, and/or receipt of any prior gene therapy (e.g., AAV) or ocular device implantation (other than Posterior chamber intraocular lens (PCIOL) placement following cataract surgery).
   rCFH Dose and Route, Regimen

rCFH was supplied in sterile, single use vials as a 10 mg/mL concentrated liquid that, if necessary, can be diluted to the appropriate concentration prior to dosing at the site. All dose administration was open label.

### Biomarkers

Aqueous humor samples were collected prior to and after receiving single IVT injections of rCFH to evaluate complement proteins and possibly inflammatory cytokine profiles to determine their potential as PD biomarkers for usefulness in a pivotal study. rCFH concentrations in aqueous humor were evaluated after completion of PD analyses, if volume permits. Imaging endpoints such as GA lesion size, and clinical endpoints such as BCVA, LLVA, and low luminance deficit (LLD), were collected as described in Tables 5 and 6.

### Example 5. Results of the Phase 2, Multiple Dose Study of Recombinant CFH in Patients with Dry AMD

Patients with geographic atrophy secondary to dry AMD were treated in multiple dose study as described in Example 4. Specifically, patients were assigned to 1 of 6 cohorts, based on subject genetic profile and location of GA. Patients in MD cohorts 1-4 (as described in this Example 4) received 250 μg rCFH either monthly or every other month (EOM), based on their initial treatment assignment. For patients in MD cohorts 1-4, dose escalation from 250 μg to 500 μg monthly was done when at least 3 patients completed at least 2 doses of rCFH at 250 μg. Patients in MD cohorts 5 and 6 received 500 μg rCFH monthly.

PK of rCFH is Dose Proportional and Supports Every Other Month Dosing

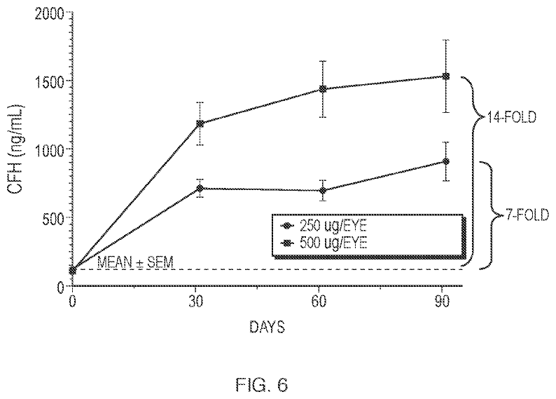

CFH concentration levels in aqueous humor (AH) were quantified. For quantification of CFH levels in AH, Luminex-Human Complement Magnetic Bead Panel 2 Kit (Millipore catalog number HCMP2MAG-19K) was used. The double capture multiplex bead-based immunoassay uses spectrally encoded magnetic beads conjugated to CFH and anti-factor H capture antibodies are used as the solid support. CFH in samples and control samples bound to the antibodies conjugated to color-coded beads. The bound CFH was detected with biotinylated anti-FH antibody producing a magnetic bead antibody-antigen-antibody double capture complex. When a reporter molecule, streptavidin-conjugated to Phycoerythrin (Streptavidin-PE) was added to the mixture, a fluorescence was generated. The fluorescence signal was read using a BioPlex Suspension Array instrument. The concentration of CFH was determined by monitoring the spectral properties of the magnetic beads and the PE Fluorescence intensity (FI). Factor B and C3 were also analyzed in this assay with specific anti-FB and anti-C3 antibody coated beads. The lower limit of quantification (LLOQ) and upper limit of quantification (ULOQ) for CFH for this assay was 1.10 ng/mL and 67.50 ng/mL, respectively. FIG. 6 is a graph showing CFH levels in aqueous humor after administration of 250 μg of rCFH monthly or 500 μg of rCFH monthly. By the indicated time points, the patients were administered either 250 μg of rCFH monthly or 500 μg of rCFH monthly. No dose escalation had occurred at the time points indicated in FIG. 6. As shown in FIG. 6, both doses achieved multiple-fold (7-fold for 250 μg to 500 μg of rCFH monthly and 14-fold for 500 μg of rCFH monthly) increase over baseline CFH levels. In addition, little difference in CFH mean Cₘₐₓ produced by administering rCFH at dose level was observed. Incremental and cumulative aqueous humor CFH AUCs were significantly higher at a dose of 500 μg monthly. The graph also shows that the pharmacokinetics of rCFH is dose proportional. Combining the results from Phase 1 clinical study, which showed that the half-life of rCFH in AH is more than 7 days and the phase 2 clinical study, the pharmacokinetics of rCFH supports a dosing frequency of every other month.

Administration of rCFH Restores Regulation to Complement in Dry AMD Patients

### Ba Concentration Levels

Quantitative measurement of Ba fragment in human aqueous humor was done using a MicroVue Ba fragment EIA Kit (Quidel, cat #A033). Based on quantitative ELISA format, a microtiter plate was coated with a mouse mAb that specifically bound to human Ba. Upon addition of standards, controls, and samples to the wells, the Ba fragments present in them bound to the immobilized antibody. A horse radish peroxidase (HRP) conjugated polyclonal anti-Ba antibody was used as the detection reagent. The amount of Ba in the samples was detected by 3,3′, 5,5′ tetramethylbenzidine dihydrochloride (TMB) substrate, a ready-to-use chromogenic substrate solution. TMB substrate generated color which was directly proportional to the amount of Ba in the samples. The LLOQ and ULOq values of Ba in this assay were 131.69 pg/mL and 2100 pg/mL, respectively.

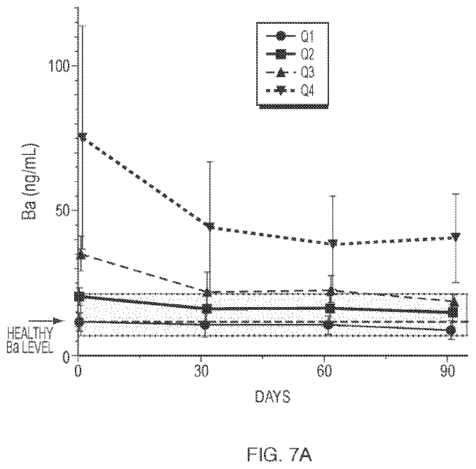

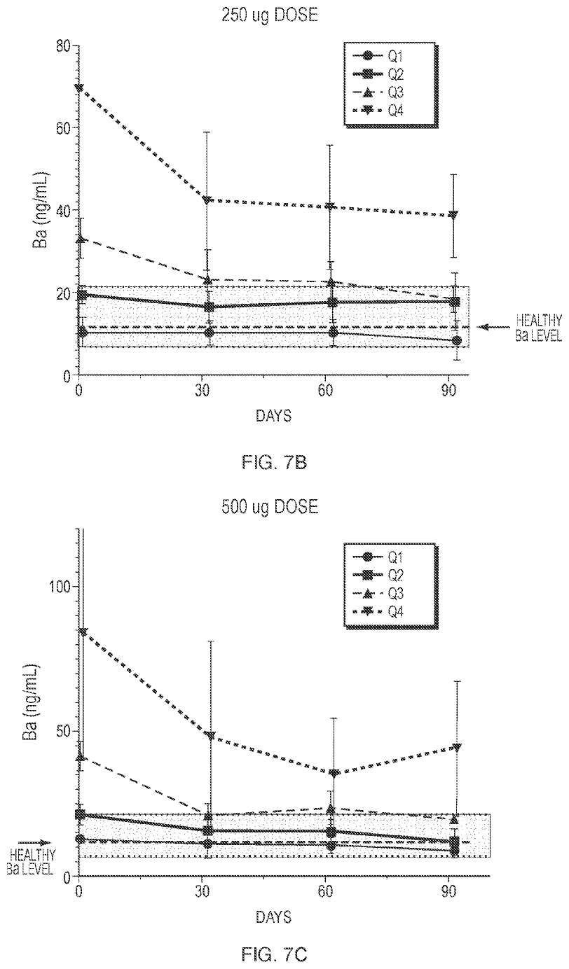

Ba levels were examined in quartile range with quartiles 1-4 having pre-treatment Ba levels in the ranges between 5.4 ng/mL and 16.65 ng/mL; between 16.91 ng/mL and 25.87 ng/mL; between 26.25 ng/mL and 44.22 ng/mL; and between 44.46 ng/mL and 165.6 ng/mL, respectively. FIGS. 7A-C are a set of graphs showing Ba concentration levels in aqueous humor after administration of either 250 μg of rCFH monthly or 500 μg of rCFH monthly (FIG. 7A); 250 μg of rCFH monthly (FIG. 7B); or 500 μg of rCFH monthly (FIG. 7C). As shown in FIG. 7A-C, administration of rCFH has a robust and prolonged impact on the Ba levels, by regulating the complement pathway, in the dry AMD patients who had an elevated initial level prior to rCFH treatment. Ba levels did not reduce below healthy levels in patients without elevated Ba levels showing that administration of rCFH did not affect the regulatory mechanism.

### C3a Concentration Levels

Quantitative measurement of C3a fragment in human aqueous humor was done using a MicroVue C3a Plus Enzyme Immunoassay Kit (Quidel, cat #A031). Briefly, a 96 well microassay plate was coated with a murine monoclonal antibody specific for human C3a. Upon addition of standards, controls, and samples to the coated plate, the antibody on the plate bound to C3a. After the incubation period, the plate was washed to remove any unbound material. Next, a horseradish peroxidase (HRP)-conjugated anti-C3a was added to each assay well where it bound to the immobilized C3a captured in the first step. When a 3,3′,5,5′ tetramethylbenzidine (TMB), a ready-to-use, chromogenic substrate solution, was added to the assay wells, the bound HRP reacted with the substrate forming a blue color. After the incubation period, the reaction was stopped and the color intensity was measured spectrophotometrically at A450. The color intensity of the reaction mixture was proportional to the concentration of C3a present in the standards, controls, and samples. Results were calculated from the generated standard curve using 5-parameter analysis. The LLOQ and ULOq values of C3a in this assay were 31.81 pg/mL and 2052.63 pg/mL, respectively.

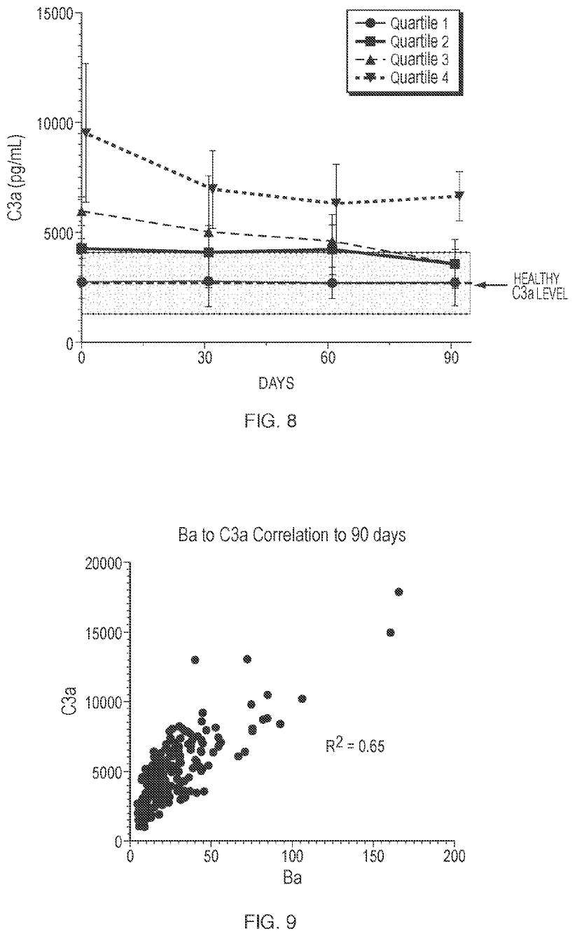

C3a levels were examined in quartile range with quartiles 1-4 having pre-treatment C3a levels in the ranges between 1097 pg/mL and 3442 pg/mL; between 3510 pg/mL and 4950 pg/mL; between 5040 pg/mL and 6968 pg/mL; and between 7022 pg/mL and 17882 pg/mL, respectively. FIG. 8 is a graph showing C3a concentration levels in aqueous humor after administration of either 250 μg of rCFH monthly or 500 μg of rCFH monthly. As shown in FIG. 8, rCFH reduced C3a levels in the patients who had an elevated initial level prior to rCFH treatment. C3a levels did not reduce below healthy levels in patients without elevated C3a levels, which shows that administration of rCFH did not affect the regulatory mechanism. FIG. 9 is a scatter dot plot showing correlation Ba and C3a concentration levels at baseline and after administration of rCFH. Analysis of the quantification levels of Ba and C3a showed that reduction in concentration levels of both biomarkers (Ba and C3a) was dependent upon baseline level of corresponding biomarker in each patient. As expected, patients with healthy levels of Ba and C3a showed no significant further reduction in Ba and C3a levels.

### Example 6. Phase 2, Multiple Dose Study of Recombinant CFH (rCFH) as an Adjunct to Standard of Care Aflibercept Therapy in Patients with Neovascular AMD (nAMD)

The safety, tolerability, immunogenicity, PK/PD, complement biomarkers, and clinical efficacy of repeat intravitreal (IVT) injection of rCFH plus standard of care (SoC) defined as aflibercept every other month (EOT), compared with sham plus SoC in subjects with nAMD are assessed in a Phase 2, multicenter and multiple-dose study.

Data from the Phase I single ascending dose study (described in Example 2) is used to inform the selection of the 500 μg/eye dose for evaluation in this Phase 2 multiple-dose study in subjects with nAMD. In this study, repeat doses of rCFH are administered by IVT to subjects with nAMD. The 500 μg/eye is based on safety data from single- and repeat-dose (up to 3 and up to 6 once monthly) IVT toxicology studies in cynomolgus monkeys, PK modeling and simulation of the profiles of rCFH after a single IVT injection in cynomolgus monkeys, and the endogenous level of CFH in human RPE (the target ocular tissue of rCFH replacement therapy in dry AMD patients), as well as data from the Phase 1 SAD study (described in Example 2).

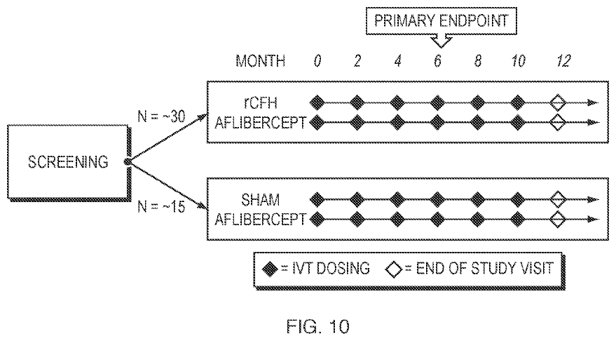

All subjects treated with rCFH will receive 500 μg rCFH IVT. One dosing regimen (EOM) will be evaluated in each treatment group. Subjects are randomized to 1 of 2 treatment groups: rCFH plus SoC or sham plus SoC. A diagram of a clinical study design is shown in FIG. 10.

### Objectives and End Points

The primary endpoint of the study is to assess rCFH plus SoC versus sham plus SoC at 6 months, and also through an additional extended 6-month dosing period. Secondary and exploratory endpoints are to assess rCFH plus SoC versus sham plus SoC at the same timepoints and may be evaluate at additional timepoints.

The primary objective of this study is evaluate the safety and tolerability of rCFH plus SoC versus sham plus SoC. The endpoint for the primary objective is treatment-related intraocular adverse events (AEs).

The secondary objectives are (1) to evaluate total CFH in aqueous humor after rCFH IVT injection; (2) to describe the effect on best corrected visual acuity (BCVA); (3) to describe the effect on macular atrophy (MA) size in subjects with MA present at baseline. The endpoints for the secondary objectives include: aqueous humor concentrations of total CFH; mean change in BCVA from baseline in Early Treatment Diabetic Retinopathy Study (ETDRS) letters; and mean change in size of MA evaluated by fundus autofluorescence (FAF) and optical coherence tomography (OCT) from baseline.

The exploratory objectives are (1) to evaluate the immunogenicity of rCFH in serum after rCFH IVT injection; (2) to describe the effect of rCFH IVT injection on biomarkers in aqueous humor; (3) to describe the effect on incidence of new MA in subjects without MA at baseline; (4) to describe the effect on choroidal neovascularization (CNV) size; (5) to describe the effect on thickness of retinal layers; (6) to describe the effect on subretinal and intraretinal fluid; (7) to describe the effect on low luminance visual acuity (LLVA); and (8) to describe the effect on National Eye Institute Visual Functioning Questionnaire 25 item Version (NEI-VFQ-25)/Minnesota Low-vision Reading Test (MNRead). The endpoints for the exploratory objectives include: generation of serum rCFH antidrug antibodies; change from baseline in complement protein, complement split products, and related cytokines protein values in aqueous humor; new MA evaluated by FAF and OCT from baseline; mean reduction from baseline in CNV size as evaluated by fluorescein angiography (FA) and OCT; mean change from baseline in center point thickness and central subfield thickness in μm as evaluated by spectral domain optical coherence tomography (SD-OCT); incidence of new fluid or disappearance of existing fluid, both subretinal and intraretinal, as evaluated by SD-OCT; mean change in LLVA from baseline in ETDRS letters; and mean change in score from baseline on NEI-VFQ-25/MNRead.

### Methodology

rCFH or sham, along with SoC aflibercept, is administered to 1 eye only, which is designed during the screening process, based on eligibility, and prior to any baseline measurements. If both eyes meet all eye-specific entry criteria, then the study eye is determined by the Investigator and the subject together. The fellow eye may be followed for comparative analyses. Detailed assessments of intraocular pressure (IOP), visual acuity (VA) and function, and retinal parameters such as CNV and MA lesion size is performed for each subject enrolled

A schedule of assessments for 6-month initial dosing period is described in Table 8.

TABLE 8
Schedule of Assessments

| | | | 48 hr Post- | | | | | | | | |
|---|---|---|---|---|---|---|---|---|---|---|---|
| | IC/EC | | dose Phone | Week | Week | Month | Month | Month | Month | Month | Month |
| | Screen ^{a} | Baseline | Call | 1 | 2 | 2 | 4 | 6 | 8 | 10 | 12 EOS |
| | Clinical Visit (C) or Phone Visit (P) | | | | | | | | | | |
| | C | C | P | P | C | C | C | C | C | C | C |
| | Study Day (First Dose = Day 1) | | | | | | | | | | |
| | −35 to | | 3 | 8 | 15 | 61 | 121 | 181 | 241 | 301 | 361 |
| | −3 | 1 | (±1) | (±2) | (±2) | (±4) | (±4) | (±4) | (±4) | (±4) | (±4) |
| Informed consent ^{b} | X | | | | | | | | | | |
| Blood sample - | X | | | | | | | | | | |
| exploratory genetics * | | | | | | | | | | | |
| Medical and Ocular | X | X | | | | | | | | | |
| History *^{c} | | | | | | | | | | | |
| Inclusion/Exclusion | X | X | | | | | | | | | |
| Criteria *^{d} | | | | | | | | | | | |
| Demographics | X | | | | | | | | | | |
| Complete Physical | X | | | | | | | | | | X |
| Examination | | | | | | | | | | | |
| ECG | X | | | | | | | | | | |
| Vital Signs *^{e} | X | X | | | X | X | X | X | X | X | X |
| Concomitant | X | X | X | X | X | X | X | X | X | X | X |
| Medications * | | | | | | | | | | | |

Treatments and AEs

| | | | | | | | | | | | |
|---|---|---|---|---|---|---|---|---|---|---|---|
| Randomization^{†} | | X | | | | | | | | | |
| Aflibercept | | SE | | | | SE | SE | SE | SE | SE | |
| SoC Administration | | | | | | | | | | | |
| Treatment (Sham or | | SE | | | | SE | SE | SE | SE | SE | |
| rCFH) Administration | | | | | | | | | | | |
| Adverse Events | X | X | X | X | X | X | X | X | X | X | X^{f} |

Ocular Assessments

| | | | | | | | | | |
|---|---|---|---|---|---|---|---|---|---|
| Complete Ophthalmic | OU | OU | OU | OU | OU | OU | OU | OU | OU |
| Examination *^{g} | | | | | | | | | |
| Intraocular | OU | OU | OU | OU | OU | OU | OU | OU | OU |
| Pressure Assessment | | | | | | | | | |
| ETDRS BCVA * | OU | OU | OU | OU | OU | OU | OU | OU | OU |
| ETDRS BCVA LLVA * | | OU | | | OU | OU | | | OU |

Imaging and Visual Function Assessments **
(Imaging assessments may be optionally performed at any in-
clinic visit if deemed medically necessary for subject safety)

| | | | | | | | | | |
|---|---|---|---|---|---|---|---|---|---|
| Color Fundus | OU | OU ⁱ | | | | OU | | | OU |
| Photography (3-Field) * | | | | | | | | | |
| Fundus Autofluorescence * | OU | OU ⁱ | | OU | OU | OU | OU | OU | OU |
| Fluorescein | OU | | | | | OU | | | OU |
| Angiography *^{j} | | | | | | | | | |
| Near Infrared | OU | OU ⁱ | | | | OU | | | OU |
| Reflectance Imaging * | | | | | | | | | |
| Optical Coherence | OU | OU ⁱ | OU | OU | OU | OU | OU | OU | OU |
| Tomography (SD-OCT) | | | | | | | | | |
| OCTA ^{k} | | OU ⁱ | | | | OU | | | OU |
| NEI-VFQ-25 *^{m} | | X | | | | X | | | X |
| MNRead *^{l} | | X | | | | X | | | X |

Other Assessments

| | | | | | | | | | |
|---|---|---|---|---|---|---|---|---|---|
| Selection of | | X | | | | | | | |
| Study Eye ^{d} | | | | | | | | | |
| Pregnancy Testing ⁿ | X | | | | | | | | X |
| Clinical Safety Laboratory | X | X | X | X | X | X | X | X | X |
| Assessments *^{o} | | | | | | | | | |
| Routine Urinalysis ^{p} | X | | | | | | | | X |
| Plasma for CFH | | X | | | | X | | | |
| Blood Sampling - ADA * | | X | | X | X | X | X | X | X |
| Aqueous Humor Sampling * | | SE | | SE | SE | SE | SE | SE | SE |

Abbreviations: ADA = antidrug antibodies; AE = adverse event; BCVA = best corrected visual acuity; CFH = complement factor H; EC = exclusion criteria; ECG = electrocardiogram; EOS = End of Study; ETDRS = Early Treatment Diabetic Retinopathy Study; hr = hour; IC = inclusion criteria; IOP = intraocular pressure; LLVA = low luminance visual acuity; MNRead = Minnesota Low-vision Reading Test; NEI VFQ-25 = National Eye Institute Visual Functioning Questionnaire-25;OCT = optical coherence tomography; OCT-A = OCT angiography; OU = oculus uterque (both eyes; eyes will be tested separately); SAE = serious adverse event; SD-OCT = spectral domain OCT; SE = study eye; SoC = standard of care (2 mg aflibercept every other month); VA = visual acuity
* Assessment obtained prior to dosing, as applicable.
** Imaging (color fundus photography, fundus autofluorescence, fluorescein angiography, near infrared reflectance imaging, SD-OCT) should be repeated atbaseline if >28 days after screening assessment, or desired by the Investigator.
^{†}Randomization may occur up to 2 days prior to the baseline visit.
^{a} Blood sampling for exploratory genetics will occur prior to first dosing.
^{b} Informed consent must be obtained before any study assessment is performed.
^{c} Medical and ocular history also includes social history and family ophthalmic history.
^{d} Some entry criteria apply only to the SE. Therefore, the SE must be identified during the screening process in order to assess entry criteria.
^{e} Vital sign measurements include systolic and diastolic blood pressures, heart rate, respiratory rate, and body temperature. The subject will be seated for at least 5 minutes before all measurements are taken. When procedures overlap and are scheduled to occur at the same timepoint, vital sign measurements should precede any blood collection.
^{f} AEs will be reported for a minimum of 30 days after the last administration of study treatment. If a subject experiences an SAE that is considered to berelated to study treatment at any time after the study, it must be reported to the Sponsor.
^{g} Complete ophthalmic examinations of both the study and fellow eye (if possible) will include an assessment of the VA and visual function using validatedassessment tools, a complete ophthalmic biomicroscopy examination including fundus examination, IOP, and ocular imaging tests to visualize the appearance and pathology of the retina and associated ocular tissues.
^{h} On dosing days, IOP assessments are obtained prior to the dose and 30 ± 15 minutes after the dose, with an additional sample at 60 ± 15 minutes if the IOP has not resolved to ≤30 mmHg at the 30-minute assessment. If the IOP continues to exceed 30 mmHg, then standard IOP-lowering interventions (per institutional guidelines) should be followed. If IOP is ≤5 mmHg, the subject should be treated per institutional guidelines. Regardless of whether applanation tonometry or a Tono-Pen ® is used to measure IOP, the same method must be used for the subject for the entirety of their participation in this study.
ⁱ These baseline imaging assessments are only required if the screening images were obtained >28 days prior to baseline. They may be redone at baseline if the Investigator deems it clinically relevant, or if there were quality issues with the screening images.
^{j} Baseline assessment of fluorescein angiography is not required, but may be assessed if the Investigator deems it clinically relevant, or if there were quality issues with the screening images.
^{k} Assessment is optional and will only be conducted if the site has approval from the Sponsor.
^{l} Assessment will be performed as per Minnesota Low-vision Reading Test (MNRead) Charts (see Altinbay et al. (2016). “The Evaluation of Reading Performance with Minnesota Low Vision Reading Charts in Patients with Age-related Macular Degeneration”. Middle East African Journal of Ophthalmology. 23 (4): 302-306).
^{m} Assessment will be performed as per National Eye Institute Visual Functioning Questionnaire 25-item Version (NEI-VFQ-25) with Near and Distance Activity Subscale Scores (developed at RAND (https://www.rand.org) under the sponsorship of the NEI).
ⁿ Highly sensitive (serum or urine) human chorionic gonadotropin pregnancy test as needed for women of childbearing potential.
^{o} Clinical safety laboratory assessments include the following: hematology (platelet count, red blood cell count, hemoglobin count, hematocrit, mean corpuscular volume, mean corpuscular hemoglobin, percent reticulocytes, and white blood cell count with differential) and clinical chemistry (blood urea nitrogen, creatinine, glucose, potassium, sodium, total calcium, aspartate aminotransferase, alanine aminotransferase, alkaline phosphatase, total and directbilirubin, total protein, standard electrolytes - magnesium, phosphate, and chloride).
^{p} Routine urinalysis includes macroscopic and microscopic examination.

### Study Duration

The minimum planned duration of each subject's participation is approximately 13 months: 1 month for screening and 12 months dosing and follow-up.

Approximately, 45 subjects (˜30 treated with rCFH plus SoC and ˜ 15 treated with sham plus SoC.

### Eligibility Criteria

### Inclusion Criteria

Subjects must meet all of the following inclusion criteria to be considered for participation: (1) at least 50 years old at the time of signed informed consent; subjects younger than 50 may be considered for participation after consultation with the Medical Monitor; (2) CNV related to nAMD with the following features, as determined by the Image Reading Center
a. Maximum CNV lesion size of 12 disc areas;
b. Subretinal hemorrhage ≤50% of lesion size

(3) must be on aflibercept treatment prior to Day 1 (with at least one aflibercept dose received within 2 months prior to Day 1) and be able to continue aflibercept at an every-other-month frequency for the duration of the study; (4) BCVA in the study eye of 24 to 75 letters using the ETDRS Chart VA Scale (approximately equivalent to Snellen VA of between 20/32 and 20/320); (5) sufficiently clear ocular media, adequate pupillary dilation, fixation to permit quality fundus imaging, and able to cooperate sufficiently for adequate ophthalmic visual function testing and anatomic assessment in the study eye; (6) understands the full nature and purpose of the study, including possible risks of study procedures, and provides informed consent prior to initiation of any study procedure; and (7) all subjects with reproductive potential must agree to use effective contraceptive methods through the end of study (EOS) visit.

### Exclusion Criteria

A subject who meets any of the following exclusion criteria is ineligible to participate in the study: (1) presence of the following ocular conditions in the study eye:
a. any active ocular disease or condition that could confound the assessment of the macula or be a contraindication to IVT injection, e.g., retinal pigment epithelium tears or rips involving the macula, macular hole (any stage, including lamellar hole), epiretinal membrane, MA or maculopathies due to any disease other than AMD, uncontrolled glaucoma (IOP of >24 mmHg when on 2 or more IOP-lowering medications), severe glaucoma (cup to disc ratio of ≥0.8), diabetic retinopathy graded as moderate nonproliferative diabetic retinopathy or worse (with or without diabetic macular edema);
b. any intraocular surgery, (with the exception of intraocular lens replacement surgery less than 3 months prior to consent) e.g., any glaucoma surgeries including microinvasive glaucoma surgery and/or any intraocular surgery requiring vitrectomy;
c. aphakia or complete absence of the posterior capsule;
d. prior corneal transplant;
e. scar or fibrosis ≥50% of CNV lesion or involving center of fovea;

(2) presence of any of the following ocular conditions—in either eye:
a. history of herpetic infection, idiopathic polypoidal choroidal vasculopathy (PCV), pathologic myopia, central serous chorioretinopathy (CSCR), adult onset foveal pattern dystrophy;
b. concurrent disease that could require medical or surgical intervention during the study period;
c. active/suspected ocular/periocular infection or active intraocular inflammation;
d. history of idiopathic or autoimmune-associated uveitis;

(3) in the opinion of the Investigator, the subject has any prior or ongoing medical condition (e.g., ocular other than wet AMD, systemic, psychiatric) or clinically significant screening laboratory value that may present a safety risk, interfere with study compliance, interfere with consistent study follow-up, or confound data interpretation throughout the longitudinal follow-up period; (4) subject has experienced a cardiovascular or cerebrovascular event within 12 months of informed consent; (5) female subjects must not be pregnant or lactating, nor plan to become pregnant during the study; (6) current use of medications known to be toxic to the lens, retina, or optic nerve (deferoxamine, chloroquine/hydroxychloroquine [Plaquenil®], tamoxifen, phenothiazines, ethambutol, digoxin, and aminoglycosides). (Current use is defined as the administration of first dose of rCFH within 5 half-lives of the prohibited medication). Past use of these medications, without history of ocular side effects, is not considered exclusionary. Use of medications that may potentially exacerbate macular edema or macular degeneration is permitted if, in the opinion of the Investigator, in consultation with the Medical Monitor, such use will not confound the interpretation of study results; or (7) use of any investigational new drug or other experimental treatment in the last 6 months, and/or receipt of any prior gene therapy (e.g. Adeno-associated virus) or ocular device implantation (other than posterior chamber intraocular lens placement after cataract surgery).

rCFH Dose and Route, Regimen

rCFH is supplied in single-use vials with a 0.25 mL fill volume at a concentration of 10 mg/mL rCFH. Aflibercept administered IVT EOM is SoC therapy. Subjects assigned to the rCFH plus SoC group are administered aflibercept (2 mg/50 μL) first, followed by rCFH (500 pg/50 μL) 15 minutes (±5 minutes) later. Study drug is not mixed in the same syringe with other products. Subjects are randomized to 1 of 2 treatment groups: rCFH plus SoC or sham plus SoC. Subjects randomized to the rCFH plus SoC treatment group receive treatment EOM (6 doses). All subjects assigned to the rCFH plus SoC treatment are treated with 500 ig rCFH per eye.

Subjects randomized to the sham plus SoC treatment group receive SoC with a sham IVT injection instead of rCFH, to keep the subject masked to their treatment assignment.

### Biomarkers

Aqueous humor is collected prior to and after receiving single IVT injections of rCFH to evaluate complement proteins and possibly inflammatory cytokine profiles to determine their potential as PD biomarkers for usefulness in a pivotal study. Total CFH concentrations in aqueous humor is evaluated. Imaging endpoints such as CNV lesion size, MA lesion size, retinal layer thickness or presence of fluid, and clinical endpoints such as BCVA, LLVA, and low luminance deficit (LLD), are collected as described in Table 8.

CFH concentration levels in human plasma can be quantified using MicroVue Factor H Enzyme Immunoassay (EIA) (Quidel, cat #A039). This is a quantitative Elisa method where the microtiter plate is coated with a mouse monoclonal antibody that binds specifically to human Factor H. When standards, controls, or specimens are added to the plate, FH binds to the immobilized anti-Factor H monoclonal antibody. Next, a horseradish peroxidase (HRP)-conjugated murine anti-Factor H antibody is added to each well as the detection reagent which binds to the FH captured on the plate. When a chromogenic enzyme substrate (TIB) is added, the bound HRP-conjugate reacts with the substrate, forming a color which is measured spectrophotometrically at A450. The color intensity of the reaction mixture is proportional to the concentration of Factor H present in the samples. Human ocular samples obtained from patient group that have been monthly administered rCFH plus SoC are expected to show elevated levels of CFH over baseline CFH levels.

### INCORPORATION BY REFERENCE

All publications and patents mentioned herein are hereby incorporated by reference in their entirety as if each individual publication or patent was specifically and individually indicated to be incorporated by reference.

### EQUIVALENTS

The present application may be embodied in other specific forms without departing from the spirit or essential characteristics thereof. The foregoing embodiments are therefore to be considered in all respects illustrative rather than limiting the application described herein. Scope of the present application is thus indicated by the appended claims rather than by the foregoing description, and all changes that come within the meaning and range of equivalency of the claims are intended to be embraced therein.

1. A method of treating a subject having an inflammatory ocular disease, disorder, or condition, the method comprising administering to the subject 50 μg, 100 μg, 250 μg, or 500 μg of CFH protein per eye, thereby treating the inflammatory ocular disease, disorder, or condition.

2. The method of claim 1, wherein the inflammatory ocular disease, disorder, or condition is selected from the group consisting of macular degeneration, diabetic retinopathy, diabetic macular edema, retinal vein occlusion, uveitis, sterile conjunctivitis, keratitis, episcleritis, and Stargardt's Disease.

3. The method of claim 2, wherein the macular degeneration is AMD.

4. The method of claim 3, wherein the AMD is dry AMD.

5. The method of claim 4, wherein the inflammatory ocular disease, disorder, or condition comprises geographic atrophy secondary to dry AMD.

6. The method of claim 1, wherein the AMD is neovascular AMD.

7. The method of claim 6, wherein the subject has been treated with a VEGF-A antagonist.

8. The method of claim 2, wherein the uveitis is anterior uveitis.

9. The method of any one of claims 1-8, wherein the CFH is administered by intravitreal injection.

10. The method of any one of claims 1-9, wherein the CFH protein comprises a recombinant CFH protein.

11. The method of any one of claims 1-10, wherein the CFH protein is administered at a dose of 50 μg per eye.

12. The method of any one of claims 1-10, wherein the CFH protein is administered at a dose of 100 μg per eye.

13. The method of any one of claims 1-10, wherein the CFH protein is administered at a dose of 250 μg per eye.

14. The method of any one of claims 1-10, wherein the CFH protein is administered at a dose of 500 μg per eye.

15. The method of any one of claims 1-14, wherein the CFH protein is administered once every four weeks, once every eight weeks, once every month, once every two months, once every three months, once every four months, once every five months, or once every six months.

16. The method of any one of claims 1-15, wherein the dose of CFH protein is 250 μg per eye, administered once every month.

17. The method of any one of claim 1-15, wherein the dose of CFH protein is 250 μg per eye, administered once every two months.

18. The method of any one of claims 1-15, wherein the dose of CFH protein is 500 μg per eye, administered once every month.

19. The method of any one of claims 1-15, wherein the dose of CFH protein is 500 μg per eye, administered once every two months.

20. The method of any one of claims 1-19, wherein the CFH protein comprises the amino acid sequence of SEQ ID NO: 2.

21. The method of any one of claims 1-20, wherein a lesion associated with the inflammatory ocular disease, disorder, or condition is observed in the subject by fundus imaging.

22. The method of any one of claims 1-21, wherein the subject carries a genetic variant associated with the inflammatory ocular disease, disorder, or condition.

23. The method of claim 22, wherein the inflammatory ocular disease, disorder, or condition is geographic atrophy, and wherein the genetic variant results in a mutation of the Tyr at amino acid position 402 of human CFH protein and is present in both alleles of the genome.

24. The method of claim 23, wherein the genetic variant results in a mutation of the Tyr at amino acid position 402 of human CFH protein to His residue and is present in both alleles of the genome.

25. The method of claim 23 or 24, wherein the genetic variant comprises rs1061170 in both alleles of the human genome.

26. The method of any one of claims 23-25, wherein the subject is negative for any one of the missense CFH mutations selected from the group consisting of R2T, L3V, R53C, R53H, S58A, D90G, D130N, R175Q, R175P, I221V, R303W, R303Q, Q400K, P503A, R567G, G650V, S890I, T956M, G1194D, R1210C, and any combination(s) thereof.

27. The method of any one of claims 23-26, wherein the subject is negative for a combination of homozygous CFH 62V, homozygous C3 102G, and homozygous CFB 32R.

28. The method of claim 22, wherein the subject is (a) homozygous for the Y402H mutation of CFH; (b) negative for any one of the missense CFH mutations selected from the group consisting of R2T, L3V, R53C, R53H, S58A, D90G, D130N, R175Q, R175P, I221V, R303W, R303Q, Q400K, P503A, R567G, G650V, S890I, T956M, G1194D, R1210C, and any combination(s) thereof; and (c) negative for a combination of homozygous CFH 62V, homozygous C3 102G, and homozygous CFB 32R.

29. The method of claim 22, wherein the subject is positive for a missense CFH mutation selected from the group consisting of R2T, L3V, R53C, R53H, S58A, D90G, D130N, R175Q, R175P, I221V, R303W, R303Q, Q400K, P503A, R567G, G650V, S890I, T956M, G1194D, R1210C, and any combination(s) thereof.

30. The method of claim 22, wherein the subject is positive for a combination of homozygous CFH 62V, homozygous C3 102G, and homozygous CFB 32R.

31. The method of any one of claims 1-30, wherein the subject is at least 50 years old.

32. The method of any one of claims 1-31, further comprising administering to the subject an effective amount of a VEGF antagonist, wherein the subject has neovascular AMD.

33. The method of claim 32, wherein the VEGF antagonist comprises aflibercept.

34. The method of claim 33, wherein the effective amount of aflibercept is 2 mg per eye.

35. The method of claim 34, wherein the aflibercept is administered once every four weeks, once every eight weeks, once every month, or once every two months.

36. The method of any one of claims 1-35, wherein the treatment slows the progression of the inflammatory ocular disease, disorder, or condition in the subject.

37. The method of any one of claims 1-36, wherein the treatment reduces the severity of the inflammatory ocular disease, disorder, or condition in the subject.

38. The method of claim 36 or 37, wherein the progression or severity of the inflammatory ocular disease, disorder, or condition is assessed by one or more methods selected from:
(a) best corrected visual acuity (BCVA) score;
(b) low luminance visual acuity (LLVA) score;
(c) AREDS 9-step severity scale score;
(d) area of geographic atrophy assessed by color fundus photography, fundus autofluorescence, optical coherence tomography, optical coherence tomography—angiography, near infrared imaging, and/or fluorescein angiography;
(e) drusen volume; and
(f) one or more retinal architecture parameters assessed by optical coherence tomography, selected from total retinal and choroidal thickness, photoreceptor layer thickness, features of nascent geographic atrophy, retinal pigment epithelium thickening, and integrity of retinal pigment epithelium layer.

39. A method for treating a subject having an inflammatory ocular disease, disorder, or condition, the method comprising:
(a) obtaining or having obtained a measurement of the protein level of at least one biomarker in an ocular sample of the subject, wherein the biomarker is selected from the group consisting of (i) complement component C3, complement factor B (CFB), complement component C5, cleavage fragments thereof, and complement factor H (CFH), (ii) proteins associated with ocular inflammation, and (iii) proteins associated with choroidal neovascularization;
(b) determining whether the protein level of the biomarker is greater than or lower than a predetermined threshold; and
(c) administering to the subject an effective amount of CFH if the protein level of the biomarker is (i) greater than or equal to the threshold if the biomarker is positively correlated with activation of the complement pathway, ocular inflammation, or choroidal neovascularization; or (ii) lower than or equal to the threshold if the biomarker is negatively correlated with activation of the complement pathway, ocular inflammation, or choroidal neovascularization,
thereby treating the inflammatory ocular disease, disorder, or condition.

40. The method of claim 39, wherein the inflammatory ocular disease, disorder, or condition is selected from the group consisting of macular degeneration, diabetic retinopathy, diabetic macular edema, retinal vein occlusion, uveitis, sterile conjunctivitis, keratitis, episcleritis, and Stargardt's Disease.

41. The method of claim 40, wherein the macular degeneration is age-related macular degeneration (AMD).

42. The method of claim 41, wherein the AMD is dry AMD.

43. The method of claim 42, wherein the inflammatory ocular disease, disorder, or condition comprises geographic atrophy secondary to dry AMD.

44. The method of claim 41, wherein the AMD is neovascular AMD.

45. The method of claim 44, wherein the subject has been treated with a VEGF-A antagonist.

46. The method of claim 40, wherein the uveitis is anterior uveitis.

47. The method of any one of claims 39-46, wherein the ocular sample comprises aqueous humor.

48. The method of any one of claims 39-47, wherein the at least one biomarker comprises a cleavage fragment of C3, CFB, or C5, and the protein level of the cleavage fragment is positively correlated with activation of the complement pathway.

49. The method of claim 48, wherein the cleavage fragment is selected from the group consisting of C3a, Ba, and C5a.

50. The method of claim 49, wherein the cleavage fragment is C3a.

51. The method of claim 50, wherein the threshold is 2 ng/mL.

52. The method of claim 49, wherein the cleavage fragment is Ba.

53. The method of claim 52, wherein the threshold is 8 ng/mL.

54. The method of any one of claims 39-53, wherein the at least one biomarker comprises CFH, and the protein level of CFH is negatively correlated with activation of the complement pathway.

55. The method of claim 54, wherein the threshold is 60 ng/mL.

56. The method of any one of claims 39-55, wherein the at least one biomarker comprises a protein associated with ocular inflammation.

57. The method of claim 56, wherein the protein associated with ocular inflammation is selected from the group consisting of IL-1β, IL-6, IL-8, IL-10, IL-18, TNF-α, CCL2, CXCL5, and Eotaxin-2, and the protein level of the biomarker is positively correlated with ocular inflammation.

58. The method of any one of claims 39-57, wherein the at least one biomarker comprises a protein associated with choroidal neovascularization.

59. The method of claim 58, wherein the protein associated with choroidal neovascularization is VEGF-A, and the protein level of the biomarker is positively correlated with choroidal neovascularization.

60. The method of any one of claims 39-59, wherein the CFH is administered by intravitreal injection.

61. The method of any one of claims 39-60, wherein the CFH comprises a CFH protein.

62. The method of claim 61, wherein the CFH protein comprises a recombinant CFH protein.

63. The method of claim 61 or 62, wherein the CFH protein is administered at a dose of 50 μg, 100 μg, 250 μg, or 500 μg per eye.

64. The method of any one of claims 61-63, wherein the CFH protein is administered once every four weeks, once every eight weeks, once every month, once every two months, once every three months, once every four months, once every five months, or once every six months.

65. The method of any one of claims 39-60, wherein the CFH comprises a vector encoding a CFH protein.

66. The method of claim 65, wherein the vector is an adeno-associated virus vector.

67. The method of any one of claims 61-66, wherein the CFH protein comprises the amino acid sequence of SEQ ID NO: 2.

68. The method of any one of claims 39-67, wherein the steps (a)-(c) are conducted after an initial administration of CFH to the subject.

69. The method of any one of claims 39-68, wherein a lesion associated with the inflammatory ocular disease, disorder, or condition is observed in the subject by fundus imaging.

70. The method of any one of claims 39-69, wherein the subject carries a genetic variant associated with the inflammatory ocular disease, disorder, or condition.

71. The method of claim 70, wherein the inflammatory ocular disease, disorder, or condition is geographic atrophy, and wherein the genetic variant results in a mutation of the Tyr at amino acid position 402 of human CFH protein and is present in both alleles of the genome.

72. The method of claim 71, wherein the genetic variant comprises rs1061170 in both alleles of the CFH gene.

73. The method of any one of claims 39-72, wherein the subject is at least 50 years old.

74. The method of any one of claims 39-73, further comprising administering to the subject an effective amount of a VEGF antagonist.

75. The method of any one of claims 39-74, wherein the treatment slows the progression of the inflammatory ocular disease, disorder, or condition in the subject.

76. The method of any one of claims 39-75, wherein the treatment reduces the severity of the inflammatory ocular disease, disorder, or condition in the subject.

77. The method of claim 75 or 76, wherein the progression or severity of the inflammatory ocular disease, disorder, or condition is assessed by one or more methods selected from:
(a) best corrected visual acuity (BCVA) score;
(b) low luminance visual acuity (LLVA) score;
(c) AREDS 9-step severity scale score;
(d) area of geographic atrophy assessed by color fundus photography, fundus autofluorescence, optical coherence tomography, optical coherence tomography—angiography, near infrared imaging, and/or fluorescein angiography;
(e) drusen volume; and
(f) one or more retinal architecture parameters assessed by optical coherence tomography, selected from total retinal and choroidal thickness, photoreceptor layer thickness, features of nascent geographic atrophy, retinal pigment epithelium thickening, and integrity of retinal pigment epithelium layer.

78. A method for selecting a subject for treatment of an inflammatory ocular disease, disorder, or condition, the method comprising:
(a) obtaining or having obtained a measurement of the protein level of at least one biomarker in an ocular sample of the subject, wherein the biomarker is selected from the group consisting of (i) complement component C3, CFB, complement component C5, cleavage fragments thereof, and CFH, (ii) proteins associated with ocular inflammation, and (iii) proteins associated with choroidal neovascularization;
(b) determining whether the protein level of the biomarker is greater than or lower than a predetermined threshold; and
(c) selecting the subject for treatment of the inflammatory ocular disease, disorder, or condition if the protein level of the biomarker is (i) greater than or equal to the threshold if the biomarker is positively correlated with activation of the complement pathway, ocular inflammation, or choroidal neovascularization; or (ii) lower than or equal to the threshold if the biomarker is negatively correlated with activation of the complement pathway, ocular inflammation, or choroidal neovascularization, wherein the treatment comprises administering to the subject CFH.

79. The method of claim 78, wherein step (a) comprises measuring the protein level of the biomarker in an ocular sample of the subject.

80. The method of claim 78 or 79, wherein the inflammatory ocular disease, disorder, or condition is selected from the group consisting of macular degeneration, diabetic retinopathy, diabetic macular edema, retinal vein occlusion, uveitis, sterile conjunctivitis, keratitis, episcleritis, and Stargardt's Disease.

81. The method of claim 80, wherein the macular degeneration is AMD.

82. The method of claim 81, wherein the AMD is dry AMD.

83. The method of claim 82, wherein the inflammatory ocular disease, disorder, or condition comprises geographic atrophy secondary to dry AMD.

84. The method of claim 80, wherein the AMD is neovascular AMD.

85. The method of claim 84, wherein the subject has been treated with a VEGF-A antagonist.

86. The method of claim 80, wherein the uveitis is anterior uveitis.

87. The method of any one of claims 78-84, wherein the ocular sample comprises aqueous humor.

88. The method of any one of claims 78-87, wherein the at least one biomarker comprises a cleavage fragment of C3, CFB, or C5, and the protein level of the cleavage fragment is positively correlated with activation of the complement pathway.

89. The method of claim 88, wherein the biomarker is selected from the group consisting of C3a, Ba, and C5a.

90. The method of claim 89, wherein the biomarker is C3a.

91. The method of claim 90, wherein the threshold is 2 ng/mL.

92. The method of claim 89, wherein the biomarker is Ba.

93. The method of claim 92, wherein the threshold is 8 ng/mL.

94. The method of any one of claims 78-93, wherein the at least one biomarker comprises CFH, and the protein level of CFH is negatively correlated with activation of the complement pathway.

95. The method of claim 94, wherein the threshold is 60 ng/mL.

96. The method of any one of claims 78-95, wherein the at least one biomarker comprises a protein associated with ocular inflammation.

97. The method of claim 96, wherein the protein associated with ocular inflammation is selected from the group consisting of IL-1β, IL-6, IL-8, IL-10, IL-18, TNF-α, CCL2, CXCL5, and Eotaxin-2, and the protein level of the biomarker is positively correlated with ocular inflammation.

98. The method of any one of claims 78-97, wherein the at least one biomarker comprises a protein associated with choroidal neovascularization.

99. The method of claim 98, wherein the protein associated with choroidal neovascularization is VEGF-A, and the protein level of the biomarker is positively correlated with choroidal neovascularization.

100. The method of any one of claims 78-99, wherein the CFH is administered by intravitreal injection.

101. The method of any one of claims 78-100, wherein the CFH comprises a CFH protein.

102. The method of claim 101, wherein the CFH protein comprises a recombinant CFH protein.

103. The method of claim 101 or 102, wherein the CFH protein is administered at a dose of 50 μg, 100 μg, 250 μg, or 500 μg per eye.

104. The method of any one of claims 101-103, wherein the CFH protein is administered once every four weeks, once every eight weeks, once every month, once every two months, once every three months, once every four months, once every five months, or once every six months.

105. The method of any one of claims 78-100, wherein the CFH comprises a vector encoding a CFH protein.

106. The method of claim 105, wherein the vector is an adeno-associated virus vector.

107. The method of any one of claims 101-106, wherein the CFH protein comprises the amino acid sequence of SEQ ID NO: 2.

108. The method of any one of claims 78-107, wherein the steps (a)-(c) are conducted after an initial administration of CFH to the subject.

109. The method of any one of claims 78-108, wherein a lesion associated with the inflammatory ocular disease, disorder, or condition is observed in the subject by fundus imaging.

110. The method of any one of claims 78-109, wherein the subject carries a genetic variant associated with the inflammatory ocular disease, disorder, or condition.

111. The method of claim 110, wherein the inflammatory ocular disease, disorder, or condition is geographic atrophy, and wherein the genetic variant results in a mutation of the Tyr at amino acid position 402 of human CFH protein and is present in both alleles of the genome.

112. The method of claim 111, wherein the genetic variant comprises rs1061170 in both alleles of the human genome.

113. The method of any one of claims 78-112, wherein the subject is at least 50 years old.

114. The method of any one of claims 78-113, wherein the treatment further comprises administering to the subject an effective amount of a VEGF antagonist.

115. The method of any one of claims 78-114, wherein the treatment slows the progression of the inflammatory ocular disease, disorder, or condition in the subject.

116. The method of any one of claims 78-115, wherein the treatment reduces the severity of the inflammatory ocular disease, disorder, or condition in the subject.

117. The method of claim 115 or 116, wherein the progression or severity of the inflammatory ocular disease, disorder, or condition is assessed by one or more methods selected from:
(a) best corrected visual acuity (BCVA) score;
(b) low luminance visual acuity (LLVA) score;
(c) AREDS 9-step severity scale score;
(d) area of geographic atrophy assessed by color fundus photography, fundus autofluorescence, optical coherence tomography, optical coherence tomography—angiography, near infrared imaging, and/or fluorescein angiography;
(e) drusen volume; and
(f) one or more retinal architecture parameters assessed by optical coherence tomography, selected from total retinal and choroidal thickness, photoreceptor layer thickness, features of nascent geographic atrophy, retinal pigment epithelium thickening, and integrity of retinal pigment epithelium layer.
